SEQUENCE LISTING (80 sequences)

SEQ ID NO: 1 - protein, 135 aa, Artificial sequence
  Consensus sequence
QVQLVQPGTAMKSLGSSLTITCRVSGDDLGSFHFGTYFXXWVRQAPGQGLEYMGGXXPXXXXXXXAXKFXGRVSISAPGVPPVLSLALTNLTYDDTATYFCARERGRHXXXKXXXXXXGXXXDXWGRGTFVRVSP

SEQ ID NO: 2 - protein, 100 aa, Artificial sequence
  consensus sequence
QSALTQPASVSGSPGQSINISCXXXXDXXSWYQQRPNGVPKLLMFDVXXRPSGVSDRFSGSHSGDTAFLTISGLQTEDEADYYCTXXPYXFGAGTKVNVL

SEQ ID NO: 3 - protein, 129 aa, artificial sequence
  consensus sequence
EVQLSESGGGFVKPGGSLRLSCEASGFTFXNYAMGWVRQAPGKGLEWVSVTXXHGXXXYFGEFVKGRFTMSRDHFIDTVYLEMNRLTVEDTAVYYCVRXXXYHEXSGYYYRAGNYFDXWGQGTLVIVSA

SEQ ID NO: 4 - protein, 107 aa, artificial sequence
  consensus sequence
DIQMTQSPSSLSASIGDRVTITCRASQDIANYLNWYQKKSGTPPKLLIFXATNLHXGVSPRFSGSGHGTHFSLTITNVQHEDFANYFCQXSFXTXGXFGQGTWVDIR

SEQ ID NO: 5 - protein, 135 aa, Homo sapiens
QVQLVQPGTAMKSLGSSLTITCRVSGDDLGSFHFGTYFMIWVRQAPGQGLEYMGGILPSTKTPTYAHKFRGRVSISAPGVPPVLSLALTNLTYDDTATYFCARERGRHFEPKNRDNLEGKFFDLWGRGTFVRVSP

SEQ ID NO: 6 - protein, 132 aa, Homo sapiens
QVQLVQSGTALKSPGSSAKLSCTVLGDEFGTYFISWIRQAPGQGLEWVGGIVPHSQLQSSAQKFHGRIIISAGAGVTPPVVSLQLTNLTFEDTGTYFCARERGRHRDPKTGDALRGRYFDFWGRGTGVSVSA

SEQ ID NO: 7 - protein, 135 aa, Homo sapiens
QLQLDQPGAAMKSLGSSLTITCRVSGDDLGSFHFGTYFMIWVRQGPGQGLEYMGGILPSTKTPTYAHKFRGRVSISAPGVPPVLSLALTNLTYDDTATYFCARERGRHFEPKNRDHLEGKFLDLWGRGTFVRVSP

SEQ ID NO: 8 - protein, 130 aa, Homo sapiens
QVQLVQSGAVVKSPGSSAKLSCRLLGDEFGTYFISWIRQAPGRGLEWVGGIVPHSQLQSSAQKFHGRFFITGGVTPPIIFLQLTNVTFEDTGTYFCARERGRHRDPKTGKSLRGRYFDFWGRGTGVTVSP

SEQ ID NO: 9 - protein, 134 aa, Homo sapiens
QPYIVQSGPAVKGAGSSVTVTCKVDGDDDGIFSFGTYFMVWVRQAPGQGLQYMGGLVPSYAPTYAHKFEGRITISAAGTPPVVSLTLLNLTNDDAATYFCARERGRHYDSKTRENRRGRYFDMWGRGTRITVSP

SEQ ID NO: 10 - protein, 134 aa, Homo sapiens
QPYIVQSGPAVKGAGSSVTVTCRVDGDDDGIFNFGTYFMVWVRQAPGQGLQYMGGLVPSYAPTYAHKFEGRITISAAGTPPVVSLTLINLTNADAATYFCARERGRHYDSKTRENRRGRYFDMWGRGTRITVSP

SEQ ID NO: 11 - protein, 130 aa, Homo sapiens
QIELRQSGAAMKSPGSSVKLSCTVLGENFGTYFISWIRQAPGQGLEWVGGIVPHSQLQSSAQKFHGRIKISGGGTPPVVSLELTNVTFDDTGTYFCARERGRHVEPKTGDRLAGRYFDIWGRGTGLRVSP

SEQ ID NO: 12 - protein, 132 aa, Homo sapiens
QVQLVQSGAAVKSPGSSAKLSCTVLGDEFGTYFISWIRQAPGQGLEWVGGIVPHSQLQSSAQRFHGRITISAGAGVTPPVVSLQLTNLTFDDAGTYFCARERGRHRDPKTGDALRGRYFDFWGRGTGISVSP

SEQ ID NO: 13 - protein, 133 aa, Homo sapiens
QVQLVQSGTALKSPGSSAKLSCTVLGDEFGTYFISWIRQAPGQGLEWVGGIVPHSQLQSSAQKFHGRIIISAGAGVTPPVVSLQLTNLTFEDTGTYFCARERGRHRDPKTGDALRGRYFDFWGRGTGLRVSTS

SEQ ID NO: 14 - protein, 132 aa, Homo sapiens
QVQLVQSGTALKSPGSSAKLSCTVLGDEFGTYFISWIRQAPGQGLEWVGGIVPHSQLQSSAQKFHGRITISAGAGVTPPVVSLQLTNLTFEDTGTYFCARERGRHRDPKTGDALRGRYFDFWGRGTGVRVST

SEQ ID NO: 15 - protein, 132 aa, Homo sapiens
QVQLVQSGTALKSPGSSAKLSCTVLGDEFGTYFISWIRQAPGQGLEWVGGIVPHSQLQSSAQKFHGRIIISAGAGVTPPVVSLQLTNLTFEDTGTYFCARERGRHRDPKTGKAPRGRYFDFWGRGTGVTVSA

SEQ ID NO: 16 - protein, 135 aa, Homo sapiens
QVQLVQPGTAMKTLGSSLTISCRVSGDDGNFHFGTYFMAWVRQVPGQGLEYMGGILPSTKSPTYAHKFRGRVSISTAGVPPVVSLALTNLTYDDTATYFCARERGRHYDSKKQENYRGKFFDLWGRGTWVRVSPT

SEQ ID NO: 17 - protein, 102 aa, Homo sapiens
QSALTQPASVSGSPGQSINISCAGRSDRVSWYQQRPNGVPKLLMFDVYRRPSGVSDRFSGSHSGDTAFLTISGLQTEDEADYYCTSHPYAFGAGTKVNVLRQ

SEQ ID NO: 18 - protein, 100 aa, Homo sapiens
QSVLTQPAFVSGSPGQSITLSCTLGTEDVSWYQQRPNSAPRLIIFDVKKRPSDVSDRFSGHNSDTAAFLTISELRPEDEAEYFCTSWPYRFGRGTKVDVA

SEQ ID NO: 19 - protein, 100 aa, Homo sapiens
QSVLTQPASVSGSPGQSINISCAGRSDRVSWYQQRPNGVPKLLLFDVYRRPSGVSDRFSGSHSDDTAFLTISGLQTDDEADYYCTSHPYAFGPGTKVNVL

SEQ ID NO: 20 - protein, 100 aa, Homo sapiens
QSVLTQPAFVSASPGQSITLSCNLGTDYVSWYQQRPSSAPRLIIFDVKNRPSGVSDRFSGYNSDTAAFLTISGLQPEDEAEYYCTSWPYNFGPGTKVDVT

SEQ ID NO: 21 - protein, 102 aa, Homo sapiens
QSVLTQPASVSGSPGQTISISCSGTFGSDSVSWYQQYPNSAPTLIIFDVHRRPSGISTRFSGSTSGDTASLTISTLHREDEAYYYCTSHPYDFGGGTYVIVL

SEQ ID NO: 22 - protein, 102 aa, Homo sapiens
QSVLTQPASVSGSPGQTISISCSGTFGSDSVSWYQQFPNSAPTLIIFDVHRRPSGISTRFSGSTSGDTASLTISTLHREDEAFYYCTSHPYDFGGGTYVIVL

SEQ ID NO: 23 - protein, 100 aa, Homo sapiens
QSVLTQPAFVSGSPGQSITISCTVGTTDVSWYQQRPNSAPRLIIFDVKKRPSGVSDRFSGSSSDNAAFLTIAGLRPEDEAEYYCTSWPYRFGRGTKVDVG

SEQ ID NO: 24 - protein, 100 aa, Homo sapiens
QSALTHPAFVSGSPGQSITLSCTLGTEDVSWYQQRPNSAPRLIIFDVRKRPSEVSDRFSGHKSDTAAFLTISDLRPEDEAEYFCTSWPYRFDRGTRLDVA

SEQ ID NO: 25 - protein, 102 aa, Homo sapiens
QSALTQPAFVSGSPGQSITLSCSLGTADVSWYQQRPNSAPRLIIFDVKKRPSDVSDRFSGHNSDTAAFVTISELRPEDEAEYFCTSWPYRFGPGTKVDVARQ

SEQ ID NO: 26 - protein, 100 aa, Homo sapiens
QSALTQPAFVSGSPGQSITLSCTLGTEDVSWYQQRPNSAPRLIIFDVKKRPSDVSDRFSGHNSDTAAFLTISELRPEDEAEYFCTSWPYRFGRGTKVDVA

SEQ ID NO: 27 - protein, 100 aa, Homo sapiens
QSALTQPAFVSGSPGQSITLSCTLGTEDVSWYQQRPNSAPRLIIFDVKKRPSDVSDRFSGHNSDTAAFLTISELRPEDEADYFCTSWPYRFDRGTKVDVA

SEQ ID NO: 28 - protein, 102 aa, Homo sapiens
QSALTQPASVSGSPGQSITISCTADSDYVSWYQQRPNSAPKLILFDVNSRPSGVSDRYSGSRSGNTASLTISGLQREDEADYFCTPWPYDFRPGTRVFVLGQ

SEQ ID NO: 29 - protein, 129 aa, Homo sapiens
EVQLSESGGGFVKPGGSLRLSCEASGFTFNNYAMGWVRQAPGKGLEWVSVTSAHGGSAYFGEFVKGRFTMSRDHFIDTVYLEMNRLTVEDTAVYYCVRVTFYHEGSGYYYRAGNYFDSWGQGTLVIVSA

SEQ ID NO: 30 - protein, 129 aa, Homo sapiens
EVQLSESGGGVVKPGGSLRLSCEASGFTFSNYAMSWVRQAPGKGLEWVSVTGGHGTYGYYGEFVKGRFTISRDHSMDTVYLQMNRLRVEDTAVYYCVRTPYYHESSGYYYRAGNYFDFWGKGTLVIVSA

SEQ ID NO: 31 - protein, 129 aa, Homo sapiens
EVQLSESGGGFVKPGGSLRLSCEASGFTFNNYAMSWVRQAPGKGLEWVSVTSAHGGSAYYGEFVKGRFTMSRDHFIDTVYLEMNRLTVEDTAVYYCVRVTFYHEGSGYYYRAGNYFDSWGKGTRVVVSA

SEQ ID NO: 32 - protein, 129 aa, Homo sapiens
EVQLSESGGGFVKPGGSLRLSCEASGFTFNNYAMSWVRQAPGKGLEWVSVTSAHGGSAYYGEFVKGRFTMSRDHFIDTVYLEMNGLTVEDTAVYYCVRVTFYHEGSGYYYRAGNYFDSWGKGTRVVVSA

SEQ ID NO: 33 - protein, 107 aa, Homo sapiens
DIQMTQSPSSLSASIGDRVTITCRASQDIANYLNWYQKKSGTPPKLLIFGATNLHHGVSPRFSGSGHGTHFSLTITNVQHEDFANYFCQQSFQTVGSFGQGTWVDIR

SEQ ID NO: 34 - protein, 107 aa, Homo sapiens
DIQMTQSPSSLSASVGDRVTITCRASQDIANYLNWYQKKSGTPPKLLIFAATNLHRGVSPRFSGSGSGTHFSLTITNVQYEDFANYFCQHSFGTIGTFGQGTWVDIR

SEQ ID NO: 35 - protein, 107 aa, Homo sapiens
DIQMTQSPSSLSASIGDRVTITCRASQDIANYLNWYQKKSGTPPKLLIFAATNLHRGVSPRFTGSGHGTHFSLTITNVQHEDFGHYFCQQSFATVGSFGQGTWVDIR

SEQ ID NO: 36 - protein, 107 aa, Homo sapiens
DIQMTQSPSSLSASIGDRVTITCRASQDIANYLNWYQKKSGTPPKLLIFAATNLHRGVSPRFTGSGHGTHFSLTITNVQHEDFGNYFCQQSFATVGSFGQGTWVDIR

SEQ ID NO: 37 - DNA, 405 bp, Homo sapiens
caggtgcagctggtgcagcccgggactgcgatgaagtctcttggatcatcactgacgatcacttgcagagtctccggagacgacctcggctctttccacttcggcacttattttatgatctgggtgcgtcaagcccctggacagggccttgagtacatgggaggcatcctcccctccacgaagacacccacttacgcgcacaaatttcgaggccgcgtctctatttccgctcccggggttccgcccgtgctgtccctcgcgctgaccaacctcacatacgacgacacggccacctacttctgtgcgcgcgagagggggcgacattttgagccaaagaacagggataatctggaaggcaaatttttcgatttatggggtcgtggcaccttcgttcgcgtctcgccg

SEQ ID NO: 38 - DNA, 396 bp, Homo sapiens
caggtgcagcttgtgcagtctgggactgcgctgaagagccctggatcttcggcgaagctctcctgcacagtccttggagatgaatttggtacttatttcataagttggattcgacaggcccctggacaaggacttgagtgggtgggaggcatcgtccctcactctcagttacagagctccgcgcagaagtttcatggtcgaatcatcattagcgcgggcgcgggcgtgactccacccgttgtgtctctacaattgaccaaccttacatttgaggacaccggcacttatttctgtgcgcgcgagaggggacgacaccgtgatccgaagacgggagatgccctccgcggccgatacttcgatttctggggccgtggcaccggcgtcagcgtctccgcg

SEQ ID NO: 39 - DNA, 405 bp, Homo sapiens
cagctgcaactggaccagcctggggctgcgatgaagtctcttggatcatcactgaccatcacttgcagagtctccggagacgacctcggctccttccacttcggcacttattttatgatctgggtgcgtcaaggccctggacagggccttgagtacatgggaggcatcctcccttccacgaagacacccacttacgcgcacaaatttcgaggccgcgtctctatttccgctcccggggttccgcccgtgttgtccctcgcactgaccaacctcacctacgacgacacggccacctatttctgtgcgcgcgagaggggccgacattttgagccaaagaacagggatcatctcgaaggcaaatttttggatctctggggtcgtggcaccttcgttcgtgtctcgccg

SEQ ID NO: 40 - DNA, 390 bp, Homo sapiens
caggtgcagttagtgcagtctggggctgtggtgaagagccctggatcttcggcgaagctctcctgcagactccttggagatgaatttggcacttatttcataagctggattcgacaggccccgggacgaggacttgagtgggtgggaggcatcgtccctcactctcaattacagagttccgcgcagaaatttcatggtcgattcttcatcaccgggggcgtgactccacccattatatttctgcaattgactaacgtaacatttgaggacaccggcacttacttctgtgcgcgcgagaggggacgacaccgcgatccgaagacgggaaaatccctccgcggccgatatttcgatttctggggccgtggcaccggcgtcaccgtctccccg

SEQ ID NO: 41 - DNA, 402 bp, Homo sapiens
cagccgtacatagtacagtctgggcctgccgtgaagggggctggatcgtcggtgaccgtcacttgcaaggtcgatggagacgacgacggcattttcagtttcggcacttatttcatggtctgggtgcgacaggcccctggacaaggacttcaatatatgggcggcctcgtcccttcctatgcacccacttacgcgcacaaatttgaaggccgaatcactatttccgcggccgggaccccgcccgtcgtgtcgctgacactgctcaacctcacaaatgacgacgcggccacttatttttgtgcgcgcgagagggggcgacattacgattcaaagactcgagaaaatcgccgtggccgctatttcgacatgtggggccggggcactcggatcactgtctccccg

SEQ ID NO: 42 - DNA, 402 bp, Homo sapiens
cagccgtacatagtacagtctgggcctgccgtgaagggggctggatcgtcggtgaccgtcacttgcagggtcgatggagacgacgacggcattttcaatttcggcacttatttcatggtctgggtgcgacaggcccctggacaaggacttcaatatatgggcggcctcgtcccttcctatgcacccacttacgcgcacaaatttgaaggccgaatcactatttccgcggccgggaccccgcccgtcgtgtccctgacactgatcaacctcacaaatgccgacgcggccacttatttttgtgcgcgcgagagggggcgacattacgattcaaagactcgagaaaatcgccgtggccgctatttcgacatgtggggccggggcactcggatcactgtctccccg

SEQ ID NO: 43 - DNA, 390 bp, Homo sapiens
cagatagaattgaggcagtctggggctgcgatgaagagccctggatcttcggtgaagctctcctgcacagtccttggagaaaatttcggcacttatttcataagttggattcgacaggcccctggacaaggacttgagtgggtgggaggcatcgtccctcattctcaattacaaagttccgcacagaagtttcatggtcgaatcaaaatcagcgggggcgggacgccgcctgttgtgtccctagaactgactaatgttacatttgacgacaccggcacctacttctgtgcgcgcgagaggggacgacacgttgaaccgaagacgggcgaccgcctcgccggccgatacttcgacatctggggccgtggcaccggactccgcgtctccccg

SEQ ID NO: 44 - DNA, 396 bp, Homo sapiens
caggtgcagctagtgcagtctggggctgcggtgaagagccctggatcttcggcgaagctctcctgcacagtccttggagatgaatttggtacttatttcataagttggattcgacaggcccccggacaaggacttgagtgggtgggaggcatcgtccctcactctcaattacagagttccgcgcagaggtttcatggtcgaatcactatcagcgcgggcgcgggcgtgactccgcccgttgtgtctctacaattgaccaatcttacatttgatgacgccggcacttatttctgcgcgcgcgagaggggacgacaccgtgatccgaagacgggagatgccctccgcggccgatatttcgatttctggggccgtggcaccggcatcagcgtctccccg

SEQ ID NO: 45 - DNA, 396 bp, Homo sapiens
caggtgcagctggtgcagtctgggactgcgctgaagagccctggatcttcggcgaagctctcctgcacagtccttggagatgaatttggtacttatttcataagttggattcgacaggcccctggacaaggacttgagtgggtgggaggcatcgtccctcactctcagttacagagctccgcgcagaagtttcatggtcgaatcatcattagcgcgggcgcgggcgtgactccacccgttgtgtctctacaattgaccaaccttacatttgaagacaccggcacttatttctgtgcgcgcgagaggggacgacaccgtgatccgaagacgggagatgccctccgcggccgatatttcgatttctggggccgtggcaccggcctcagagtctccacg

SEQ ID NO: 46 - DNA, 396 bp, Homo sapiens
caggtgcagctggtgcagtctgggactgcgctgaagagccctggatcttcggcgaagctctcctgcacagtccttggagatgaatttggtacttacttcataagctggattcgacaggcccctggacaaggacttgagtgggtgggaggcatcgtccctcactctcagttacagagttccgcgcagaagtttcatggtcgaatcaccattagcgcgggcgcgggcgtgactccacccgttgtgtctctacaattgaccaaccttacatttgaggataccggcacttatttctgtgcgcgcgagaggggacgacaccgtgatccgaagacgggagatgccctccgcggccgatatttcgatttctggggccgtggcaccggcgtcagggtctccacg

SEQ ID NO: 47 - DNA, 396 bp, Homo sapiens
caggtgcagctggtgcagtctgggactgcgctgaagagccctggatcttcggcgaagctctcctgcacagtccttggagatgaatttggtacttatttcataagttggattcgacaggcccctggacaaggacttgagtgggtgggaggcatcgtccctcactctcagttacagagctccgcgcagaagtttcatggtcgaatcatcattagcgcgggcgcgggcgtgactccacccgttgtgtctctacaattgaccaaccttacatttgaggacaccggcacttatttctgtgcgcgcgagaggggacgacaccgtgatccgaagacgggaaaagccccccgcggccggtatttcgatttctggggccgtggcaccggcgtcaccgtctccgcg

SEQ ID NO: 48 - DNA, 402 bp, Homo sapiens
caggtgcagctggtgcagcctgggactgcgatgaagactctgggatcatcactgacgatctcctgtagggtttctggagacgacggcaacttccatttcgggacttattttatggcttgggtgcggcaagtccctggacaaggccttgaatatatgggagggatcctcccttccaccaaatcgcccacctacgcgcacaaatttcgaggccgggtctctatttccactgccggggttccgcccgtagtgtccctcgctctgaccaatctcacatacgacgacacggccacttatttttgtgcgcgagagagggggcgacactatgactcaaaaaaacaggaaaattatcgcggcaaatttttcgatctttggggccgtggcacctgggtccgagtttcgcca

SEQ ID NO: 49 - DNA, 300 bp, Homo sapiens
cagtctgccctgactcagccggcctccgtgtctgggagtccgggtcagtcgattaacatctcctgcgctggacgttctgaccgtgtctcctggtaccaacaacgcccgaacggcgtccccaaattattaatgttcgatgtctaccgtcgaccgtctggtgtttctgatcgcttctctggctcgcattccggcgacacggcctttttgaccatctctgggctccagacggaggacgaggctgactactattgtacctcacatccttatgccttcggggctgggaccaaggtcaatgtcctc

SEQ ID NO: 50 - DNA, 300 bp, Homo sapiens
cagtctgtgctgactcagcctgccttcgtgtctggttctccggggcagtccatcaccctctcgtgcactttaggaactgaggatgtctcttggtatcaacaacggccaaacagcgccccccgactaataatttttgatgtcaaaaagagaccgtcggatgtctctgatcgcttctctggccacaactctgacactgcggccttcctgaccatctctgaactccggcctgaggacgaggctgagtatttttgcacctcatggccttatcgtttcggccgtgggaccaaggtcgatgtcgca

SEQ ID NO: 51 - DNA, 300 bp, Homo sapiens
cagtctgtgctgactcagccggcctccgtgtctgggagtccgggtcagtcgattaacatctcctgcgctggccgttctgaccgtgtctcctggtaccaacagcgcccaaacggcgtccccaaattattactcttcgatgtctaccgtcgaccgtcgggtgtttctgatcgcttctctggctcccattcggacgacacggcctttttgaccatctctgggctccagacggacgacgaggctgactactattgtacctcacatccttatgccttcgggcctgggaccaaggtcaatgtcctc

SEQ ID NO: 52 - DNA, 300 bp, Homo sapiens
cagtctgtgctgactcagcctgccttcgtgtctgcttctccggggcagtccatcaccctctcgtgcaatttagggactgactatgtctcttggtatcaacaacggccaagcagcgccccccgactaataatttttgatgtcaaaaatcgaccgtcgggtgtctctgatcgcttctctggttacaactctgacactgcggccttcctgaccatctctggactccagcctgaggacgaggctgaatattattgcacctcatggccttataatttcggccctgggaccaaggtcgatgtcaca

SEQ ID NO: 53 - DNA, 306 bp, Homo sapiens
cagtctgtgctgactcagccagcctctgtgtctgggtctccgggtcagaccatctccatctcctgcagtgggactttcggctctgactctgtgtcttggtatcaacaatacccgaattccgcccccacacttatcatttttgatgtccaccgtcgaccctcaggcatttctactcgcttctctggctccacgtctggcgacacagcctccctcaccatctctacactccacagagaggacgaggcctattattactgcacatcgcatccctatgatttcggaggtggcacctacgtcattgtccta

SEQ ID NO: 54 - DNA, 306 bp, Homo sapiens
cagtctgtcctgactcagccagcctctgtgtctgggtctccgggtcagaccatctccatctcctgcagtgggactttcggctctgactctgtgtcttggtatcaacaattcccaaattccgcccccacacttatcatttttgatgtccaccgtcgaccctcaggcatttctactcgcttctctggctccacatctggcgacacagcctccctcaccatctctacactccacagagaggacgaggccttttattactgcacatcgcatccctatgatttcggaggtggcacctacgtcattgtccta

SEQ ID NO: 55 - DNA, 300 bp, Homo sapiens
cagtctgtgctgactcagcctgccttcgtgtctggttctccggggcagtcaatcaccatttcgtgcactgttggtactaccgatgtctcctggtaccaacaacggccgaacagcgccccccgactgataatttttgatgtcaaaaaacgaccgtcgggtgtctctgatcgcttctctggctccagttctgacaacgcggccttcctgaccatcgctggactccggcctgaggacgaagctgaatattattgcacttcctggccttatcgtttcggccgtgggaccaaggtcgatgtcggg

SEQ ID NO: 56 - DNA, 301 bp, Homo sapiens
cagtctgccctgactcatcctgccttcgtgtctggttctccggggcagtcaatcaccctctcgtgcactttaggaacagaggatgtctcttggtatcaacaacgaccaaacagcgccccccgactaataatttttgatgtcagaaaacggccgtcggaggtctctgatcgcttctcaggccacaagtctgacactgcggccttcctgaccatctctgacctccggcctgaagacgaggctgagtatttttgcacctcatggccttatcgtttcgaccgtgggacgagactcgatgtcgcac

SEQ ID NO: 57 - DNA, 300 bp, Homo sapiens
cagtctgccctgactcagcctgccttcgtgtctggttctccgggtcagtccatcaccctctcgtgctctttaggaactgcggatgtctcttggtatcaacaacggccaaacagcgccccccgactaataatttttgatgtcaaaaagagaccgtcggatgtctctgatcgcttctctggtcacaactctgacactgcggccttcgtgaccatctctgaactccggcctgaggacgaggctgaatatttttgcacctcatggccttatcgtttcggccctgggaccaaggtcgatgtcgca

SEQ ID NO: 58 - DNA, 301 bp, Homo sapiens
cagtctgccctgactcagcctgccttcgtgtctggttctccggggcagtccatcaccctctcgtgcactttaggaactgaggatgtctcttggtatcaacaacggccaaacagcgccccccgactaataatttttgatgtcaaaaagagaccgtcggatgtctctgatcgcttctctggccacaactctgacactgcggccttcctgaccatctctgaactccggcctgaggacgaggctgagtatttttgcacctcatggccttatcgtttcggccgtgggaccaaggtcgatgtcgcac

SEQ ID NO: 59 - DNA, 300 bp, Homo sapiens
cagtctgccctgactcagcctgccttcgtgtctggttctccggggcagtccatcaccctctcgtgcactttaggaactgaggatgtctcttggtatcaacaacggccaaacagcgccccccgactaataatttttgatgtcaaaaagagaccgtcggatgtctctgatcgcttctctggccacaactctgacactgcggccttcctgaccatctctgaactccggcctgaggacgaggctgactatttttgcacctcatggccttatcgtttcgaccgtgggaccaaggtcgatgtcgca

SEQ ID NO: 60 - DNA, 300 bp, Homo sapiens
cagtctgccctgactcagcctgcctccgtgtctgggtctccggggcagtcgattaccatctcctgcactgcagactctgactatgtctcctggtatcaacaacgcccgaacagcgcccccaaactcattctttttgatgtcaatagtcgaccctcaggagtttctgatcgatactctggctccaggtcaggcaacacggcctccctgaccatctctggactccagcgtgaggacgaggctgattatttctgcaccccctggccttatgacttcagacctgggacgagagtctttgtctta

SEQ ID NO: 61 - DNA, 387 bp, Homo sapiens
gaggtgcagttatcagagtcggggggaggcttcgtaaagccgggggggtccctgagactctcctgtgaggcctctggattcaccttcaataattatgccatgggttgggtccgccaggctccagggaaggggctggagtgggtctctgtgacgagtgctcatggtggtagtgcatactttggagaattcgtgaagggccgcttcaccatgtccagagaccacttcatagacacggtctacttggaaatgaacagactgacagtcgaggacacggccgtctactactgcgtcagagtgacattttaccatgagggcagtggctactattatcgtgccgggaattactttgattcctggggccagggaaccctagtcatcgtctccgca

SEQ ID NO: 62 - DNA, 387 bp, Homo sapiens
gaggtgcagttatcagaatccgggggaggcgtcgtaaagcctggggggtccctgagactctcctgtgaggcctctggattcaccttcagtaattatgccatgagttgggtccgccaggctccagggaaggggctggagtgggtctctgtgacgggtggtcatggtacttatggatattatggagaattcgtgaagggccggttcaccatctccagagaccattccatggacacggtctatttgcaaatgaacagactgagagtcgaggacacggccgtctactactgcgtcagaacgccatattaccatgagtcaagtggctactattatcgtgccggtaattactttgacttttggggcaagggaactctagtcatcgtctccgca

SEQ ID NO: 63 - DNA, 387 bp, Homo sapiens
gaggtgcagttatcagagtcgggtggaggcttcgtaaagccgggggggtccctgagactctcctgtgaggcctctggattcaccttcaataattatgccatgagttgggtccgccaggctccagggaaggggctggagtgggtctctgtgacgagtgctcatggtggtagtgcatactatggagaattcgtgaagggccggttcaccatgtccagagaccatttcatagacacggtctacttggaaatgaacagactgacagtcgaggacacggccgtctactactgcgtcagagtgacattttaccatgagggcagtggctactattaccgtgccggtaattactttgactcctggggcaagggaacccgagtcgtcgtctccgca

SEQ ID NO: 64 - DNA, 387 bp, Homo sapiens
gaggtgcagttatcagagtcgggtggaggcttcgtaaagccgggggggtccctgagactctcctgtgaggcctctggattcaccttcaataattatgccatgagttgggtccgccaggctccagggaaggggctggagtgggtctctgtgacgagtgctcatggtggtagtgcatactatggagaattcgtgaagggccggttcaccatgtccagagaccatttcatagacacggtctatttggaaatgaacggactgacagtcgaggacacggccgtctactactgcgtcagagtgacattttaccatgagggcagtggctactattaccgtgccggtaattactttgactcatggggcaagggaacccgggtcgtcgtctccgca

SEQ ID NO: 65 - DNA, 321 bp, Homo sapiens
gacatccagatgacccagtctccatcctccctgtctgcatctataggggacagagtcaccataacttgtcgggcgagtcaggacattgccaactatttaaattggtatcagaagaaatcagggactccccctaaactcctcatctttggtgcgaccaacttgcaccatggggtgtctccgaggttcagtggcagtggacatgggacgcatttcagtctcaccatcaccaatgttcaacatgaagacttcgctaattatttttgtcaacagagttttcaaaccgttgggtcattcggccaagggacctgggtggacatcaga

SEQ ID NO: 66 - DNA, 321 bp, Homo sapiens
gacatccagatgacccagtctccatcttccctgtctgcatctgtaggagacagagtcaccattacttgtcgggcaagtcaggacattgccaactatttaaattggtatcagaagaaatcagggacaccccctaaactcctcatctttgctgcgaccaacttacaccgtggggtgtctccgaggttcagtggcagtggatctgggacacatttcagtctcaccatcaccaatgtgcaatatgaagacttcgctaattatttttgtcagcatagttttggaaccattgggacattcggccaagggacctgggtggacatcaga

SEQ ID NO: 67 - DNA, 321 bp, Homo sapiens
gacatccagatgacccagtctccatcctccctgtctgcatctataggggacagagtcaccataacttgtcgggcgagtcaggacattgccaattatttaaattggtatcagaagaaatcagggactccccctaaactcctcatctttgctgcgaccaacttgcaccgtggggtgtctccgaggttcactggcagtggacatgggacgcatttcagtctcaccatcaccaatgtgcaacatgaagacttcggtcattatttttgtcaacaaagttttgcaaccgttgggtcattcggccaagggacctgggtggacatcaga

SEQ ID NO: 68 - DNA, 321 bp, Homo sapiens
gacatccagatgacccagtctccatcctccctgtctgcatctataggggacagagtcaccataacttgtcgggcgagtcaggacattgccaactatttaaattggtatcagaagaaatcagggactccccctaaactcctcatctttgctgcgaccaacttgcaccgtggggtgtctccgaggttcactggcagtggacatgggacgcatttcagtctcaccatcaccaatgtgcaacatgaagacttcggtaattatttttgtcaacagagttttgcaaccgttgggtcattcggccaagggacctgggtggacatcaga

SEQ ID NO: 69 - protein, 98 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCAR

SEQ ID NO: 70 - protein, 17 aa, Homo sapiens
YWYFDLWGRGTLVTVSS

SEQ ID NO: 71 - protein, 99 aa, Homo sapiens
QSALTQPASVSGSPGQSITISCTGTSSDVGGYNYVSWYQQHPGKAPKLMIYEVSNRPSGVSNRFSGSKSGNTASLTISGLQAEDEADYYCSSYTSSSTL

SEQ ID NO: 72 - protein, 12 aa, Homo sapiens
YVFGTGTKVTVL

SEQ ID NO: 73 - protein, 98 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 74 - protein, 95 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 75 - protein, 15 aa, Homo sapiens
YFDYWGQGTLVTVSS

SEQ ID NO: 76 - protein, 12 aa, Homo sapiens
WTFGQGTKVEIK

SEQ ID NO: 77 - protein, 458 aa, artificial sequence
  Modified antibody heavy chain
QLVQSGAEVKKPGSSVKVSCKASGGTFSSYAISWVRQAPGQGLEWMGGIIPIFGTANYAQKFQGRVTITADESTSTAYMELSSLRSEDTAVYYCARERGRHFEPKNRDNLEGKFFDLWGRGTFVRVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 212 aa, artificial sequence
  Modified antibody light chain
QSALTQPASVSGSPGQSITISCTGTSSDVGGYNYVSWYQQHPGKAPKLMIYEVSNRPSGVSNRFSGSKSGNTASLTISGLQAEDEADYYCTSHPYAFGAGTKVNVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 79 - protein, 459 aa, Artificial sequence
  Modified antibody heavy chain
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVTFYHEGSGYYYRAGNYFDSWGQGTLVIVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 214 aa, artificial sequence
  modified antibody light chain
DIQMTQSPSSLSASVGDRVTITCRASQSISSYLNWYQQKPGKAPKLLIYAASSLQSGVPSRFSGSGSGTDFTLTISSLQPEDFATYYCQQSFQTVGSFGQGTWVDIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2015/0044137 A1
(43) Pub. Date: Feb. 12, 2015

(54) NEUTRALIZING ANTIBODIES TO HIV-1 AND THEIR USE

(71) Applicant: The United States of America, as represented by the Secretary, Dep. of Health Care Human Services, Bethesda, MD (US)

(72) Inventors: John R. Mascola, Rockville, MD (US); Rebecca M. Lynch, Washington, DC (US); Xueling Wu, Potomac, MD (US); Mark Connors, Bethesda, MD (US); Tongqing Zhou, Boyds, MD (US); Joseph Casazza, McLean, VA (US); Peter D. Kwong, Washington, DC (US)

(21) Appl. No.: 14/386,920

(22) PCT Filed: Mar. 15, 2013
(86) PCT No.: PCT/US2013/032070

§ 371 Date: Sep. 22, 2014

Related U.S. Application Data
(63) Continuation-in-part of application No. PCT/US2012/030465, filed on Mar. 23, 2012.
(60) Provisional application No. 61/698,487, filed on Sep. 7, 2012.

(51) Int. Cl. C07K 16/10; G01N 33/569; A61K 45/06; A61K 39/42; A61K 49/00

(52) U.S. Cl. CPC C07K 16/1063; A61K 39/42; A61K 49/0004; A61K 45/06; G01N 33/56983; C07K 2317/31; A61K 2039/505
USPC 424/9.1; 530/388.35; 530/387.3; 530/391.7; 530/391.3; 536/23.53; 424/148.1; 514/44 R; 435/188; 435/320.1; 435/339.1; 435/5

(57) ABSTRACT

Neutralizing antibodies that specifically bind to HIV-1 gp120 and antigen binding fragments of these antibodies are disclosed. Nucleic acids encoding these antibodies, vectors and host cells are also provided. Methods for detecting HIV using these antibodies are disclosed. In addition, the use of these antibodies, antigen binding fragment, nucleic acids and vectors to prevent and/or treat an HIV infection is disclosed.

### RELATED APPLICATIONS

This claims the benefit of U.S. Provisional Application No. 61/698,487, filed Sep. 7, 2012, and International Application No. PCT/US2012/030465, which was filed at the United States receiving Office on Mar. 23, 2012. Each of the prior applications is incorporated by reference herein in its entirety.

### FIELD

This relates to broadly neutralizing monoclonal antibodies that bind to the CD4 binding site of human immunodeficiency virus (HIV)-1 gp120, their use, and methods of identifying these broadly neutralizing monoclonal antibodies.

### BACKGROUND

Human Immunodeficiency Virus (HIV) infection, and the resulting Acquired Immunodeficiency Syndrome (AIDS), remain threats to global public health, despite extensive efforts to develop anti-HIV therapeutic agents. Some HIV-infected individuals eventually develop broadly neutralizing antibodies (bNAbs), which neutralize a large panel of HIV viruses. These individuals show delayed development of AIDS, even in the absence of any treatment for HIV infection.

One previously characterized HIV-1 neutralizing mAb, called b12, can bind to a site on gp120 that is required for viral attachment to its primary cellular receptor, CD4. mAb b12 was derived from a phage display library, a process which makes it impossible to know if the antibody was naturally present in an infected person, or was the result of a laboratory combination of antibody heavy and light chains. b12 can neutralize about 75% of Glade B strains of HIV-1 (those most common in North America), but it neutralizes less than 50% of other strains of HIV-1 found worldwide. Another neutralizing antibody, VRC01, binds to the CD4 binding site of gp120 by partially mimicking the structure of the HIV-1 receptor molecule CD4. Several antibodies similar to VRC01 have been isolated. All the VRC01-like (VRC01 class) of antibodies adopt a structural mode of recognition that involves mimicking the CD4 structure and all VRC01-like antibodies derive from the IgGVH1-2 gene. However, there is a need to develop additional neutralizing antibodies for HIV-1 with different structural recognition.

### SUMMARY

Isolated monoclonal neutralizing antibodies that specifically bind HIV-1 gp120 are provided herein. Also disclosed herein are compositions including these antibodies that specifically bind gp120, nucleic acids encoding these antibodies, expression vectors comprising the nucleic acids, and isolated host cells that express the nucleic acids. The antibodies include a heavy chain variable domain and a light chain variable domain.

Several embodiments include an isolated monoclonal antibody comprising a heavy chain variable region and optionally include a light chain variable region, wherein the heavy chain variable region comprises a heavy chain complementarity determining region (HCDR) 3, of the heavy chain variable region amino acid sequence set forth as SEQ ID NO: 1, wherein the antibody specifically binds to an epitope on the surface of gp120 and is neutralizing. Additional embodiments include an isolated monoclonal antibody comprising a heavy chain variable region and a light chain variable region, wherein the heavy chain variable region comprises a heavy chain complementarity determining region (HCDR) 1, a HCDR2, and a HCDR3 of the heavy chain variable region amino acid sequence set forth as SEQ ID NO: 1, wherein the antibody specifically binds to an epitope on the surface of gp120 and is neutralizing. Several embodiments include an isolated monoclonal antibody comprising a heavy chain variable region and a light chain variable region, wherein the heavy chain variable region comprises a heavy chain complementarity determining region (HCDR) 1, a HCDR2, and a HCDR3, corresponding to Kabat positions 31-35B, 50-65 and 95-102, respectively, of the amino acid sequence set forth as SEQ ID NO: 1, wherein the antibody specifically binds to an epitope on the surface of gp120 and is neutralizing. In some embodiments, the light chain variable region of the isolated monoclonal antibody comprises a light chain complementarity determining region (LCDR) 1, a LCDR2 and an LCDR3 of the light chain variable region amino acid sequence set forth as SEQ ID NO: 2. In some embodiments, the light chain variable region of the isolated monoclonal antibody comprises a light chain complementarity determining region (LCDR) 1, a LCDR2 and an LCDR3 corresponding to Kabat positions 24-34, 50-56, and 89-97, respectively, of the amino acid sequence set forth as SEQ ID NO: 2.

Additional embodiments include an isolated monoclonal antibody comprising a heavy chain variable region and a light chain variable region, wherein the heavy chain variable region comprises a heavy chain complementarity determining region (HCDR) 1, a HCDR2, and a HCDR3 of the amino acid sequence set forth as SEQ ID NO: 3, wherein the antibody specifically binds to an epitope on the surface of gp120 and is neutralizing. In some embodiments the isolated monoclonal antibody includes a heavy chain variable region and a light chain variable region, wherein the heavy chain variable region comprises a heavy chain complementarity determining region (HCDR) 1, a HCDR2, and a HCDR3, corresponding to Kabat positions 31-35B, 50-65 and 95-102, respectively, of the amino acid sequence set forth as SEQ ID NO: 3; and wherein the antibody specifically binds to an epitope on the surface of gp120 and is neutralizing. In some embodiments, the light chain variable region of the isolated monoclonal antibody comprises a light chain complementarity determining region (LCDR) 1, a LCDR2 and an LCDR3 of the light chain variable region amino acid sequence set forth as SEQ ID NO: 4. In some embodiments, the light chain variable region of the isolated monoclonal antibody comprises a light chain complementarity determining region (LCDR) 1, a LCDR2 and an LCDR3 corresponding to Kabat positions 24-34, 50-56, and 89-97, respectively, of the amino acid sequence set forth as SEQ ID NO: 4.

Further embodiments include an isolated monoclonal antibody comprising a heavy chain variable region and a light chain variable region, wherein the heavy chain variable region is at least 80% identical to SEQ ID NO: 5 and the light chain variable region is at least 80% identical to SEQ ID NO: 17, wherein the antibody specifically binds gp120 and is neutralizing. In several such embodiments, the heavy chain variable domain is a clonal variant from donor 44, wherein the heavy chain is encoded by a V 1-69 gene, and a VJ-2 J from donor 44. In further embodiments, the light chain variable domain is a clonal variant from donor 44, wherein the light chain is encoded by a LV2-14 V gene and a LJ-1 J gene.

Some embodiments include an isolated monoclonal antibody comprising a heavy chain variable region and a light chain variable region, wherein the heavy chain variable region is at least 80% identical to SEQ ID NO: 29 and the light chain variable region is at least 80% identical to SEQ ID NO: 133, wherein the antibody specifically binds gp120 and is neutralizing. In several such embodiments, the heavy chain variable domain is a clonal variant from donor 200-384, with a heavy chain encoded by a VH3-23 V gene and a VJ-4 J gene. In further embodiments, the light chain variable domain is a clonal variant from donor 200-384, with a light chain encoded by a KV1-39 V gene and a KJ-1 J gene.

The antibodies and compositions disclosed herein can be used for a variety of purposes, such as for detecting an HIV-1 infection or diagnosing AIDS in a subject. These methods can include contacting a sample from the subject diagnosed with HIV-1 or AIDS with a human monoclonal antibody that specifically binds gp120, and detecting binding of the antibody to the sample. An increase in binding of the antibody to the sample relative to binding of the antibody to a control sample confirms that the subject has an HIV-1 infection and/or AIDS. In some embodiments, the methods further comprise contacting a second antibody that specifically binds gp120 with the sample, and detecting binding of the second antibody. In some non-limiting examples an increase in binding of the antibody to the sample relative to a control sample detects HIV-1 in the subject. In some non-limiting examples, the antibody specifically binds soluble gp120 in the sample. I n some embodiments, the methods further comprise contacting a second antibody that specifically recognizes the gp120 specific antibody with the sample and detecting binding of the second antibody.

In further embodiments, a method is disclosed for preventing an HIV infection in a subject. The methods include administering a therapeutically effective amount of one or more of the monoclonal antibodies disclosed herein, or a nucleic acid molecule encoding the antibody, to the subject, for example to a subject at risk of HIV infection.

In additional embodiments, a method is disclosed for treating a subject with an HIV infection, such as, but not limited to, a subject with AIDS. The methods include administering a therapeutically effective amount of one or more of the monoclonal antibodies disclosed herein or a nucleic acid molecule encoding the antibody.

The foregoing and other features and advantages of the invention will become more apparent from the following detailed description of a several embodiments which proceeds with reference to the accompanying figures.

### BRIEF DESCRIPTION OF THE FIGURES

FIG. 1 is a table showing the identification of a class of antibodies with a new CD4 binding site. The heavy chain of VRC13 derives from the 1-69*01 heavy chain gene and has an IMGT CDR3 length of 23 amino acids. The light chain of VRC13 derives from the 1-39*01 light chain gene. The heavy chain of VRC16 derives from the 3-23*01 heavy chain gene and has an IMGT CDR3 length of 22 amino acids.

FIG. 2 is a set of digital images of crystals of VRC13 bound to gp120 and VRC16 bound to gp120.

FIG. 3 is a set of ribbon diagrams illustrating the crystal structures of the VRC13, VRC16, and VRC01 antibodies in complex with HIV-1 gp120.

FIG. 4 is a set of diagrams illustrating that VRC13 and VRC16 target the CD4-Binding Site on HIV-1 gp120. Epitopes of VRC13 and VRC01 overlap at the initial site of CD4 attachment, but VRC13 extends its interaction into the b19 area on HIV-1 gp120.

FIG. 5 is a set of ribbon diagrams illustrating that VRC13 and VRC01 have different angles of approach to HIV-1 gp120.

FIG. 6 is a set of ribbon diagrams showing that VRC13 only uses the heavy chain to engage gp120.

FIG. 7 is a schematic diagram showing CDR H3-mediated interactions in VRC13.

FIG. 8 is a schematic diagram showing CDR H3-mediated interactions in VRC16.

FIG. 9 is a table showing that VRC13 and VRC16 primarily use CDR H3 to engage gp120.

FIG. 10 is a table of the hydrogen bonds between VRC13 heavy chain and gp120.

FIG. 11 is a table of the salt bridges between VRC13 heavy chain and gp120.

FIG. 12 is a set of ribbon diagrams showing VRC13 conserved the Arg_{Antibody}: Asp368_{gp120} salt bridges of VRC01.

FIG. 13 is a comparison of VRC13 and VRC01 heavy chains, illustrating that that a new modality implicates an Alternative Elicitation Pathway.

FIG. 14 shows the schema for isolating the specific B-cells that produced VRC16 and its clonal relatives. PBMC from an HIV-1 infected donor C38 were incubated with the probe RSC3 (specific for CD4bs antibodies) and the ΔRSC3 protein (which knocks out CD4bs antibody binding). Flow cytometry was performed to isolate memory (IgG+) B-cells and to further identify B-cells that bind RSC3 but not ΔRSC3. A total of 19 B-cells were deposited as a single cell per well, into 96-well plates.

FIGS. 15A and 15B are a set of tables showing the germline V, D and J genes for the heavy and light chains of the VRC16, VRC16b, VRC16c, and VRC16d antibodies, as well as the length of the CDR3 junction region and V gene mutation frequency. All four clonally related antibodies use the same germline genes and have the same length CDR3s.

FIG. 16 is a graph showing that VRC16 and clonally related mAbs bind to Yu2 but not to the Yu2 gp120 D368R CD4bs knockout mutant, indicating the CD4bs specificity..

FIG. 17 is a graph showing VRC16 and clonally related mAbs bind to the RSC3 probe but not to the ΔRSC3CD4bs knockout mutant, indicating the CD4bs specificity.

FIG. 18 is a set for graphs illustrating that that mAb VRC16 and CD4 cross-compete (block) with each other to bind to the Yu2 gp120 HIV-1 Env protein. These data confirm that VRC16 is directed to the CD4bs.

FIG. 19 shows the schema for isolating the specific B-cells that produced VRC13 and its clonal relatives. PBMC from Pt44 were incubated with a probe RSC3 (specific for CD4bs antibodies) and the RSC3dI371 protein (which knocks out CD4bs antibody binding). Both proteins are biotin labeled. Flow cytometry was performed to isolate memory (IgG+) B-cells and to further identify B-cells that bind RSC3 but not the d371 mutant. These B-cells were deposited as a single cell per well, into 96-well plates.

FIG. 20 is a table showing the germline V, D, and J genes for the heavy and light chains of the VRC13, VRC13b, VRC13c, VRC13d, VRC13e, VRC13f, VRC13g, VRC13h, VRC14, VRC14b, VRC14c, and VRC15 antibodies, as well as the length of the CDR3 junction region and V gene mutation frequency. All 12 clonally related antibodies use the same germline genes and have the same length CDR3s.

FIGS. 21A-21B show protein sequence alignments for the heavy and light chain of VRC13 and the related clones. The heavy chains of the VRC13 (SEQ ID NO: 5), VRC13b (SEQ ID NO: 6), VRC13c (SEQ ID NO: 7), VRC13d (SEQ ID NO: 8), VRC13e (SEQ ID NO: 9), VRC13f (SEQ ID NO: 10), VRC13g (SEQ ID NO: 11), VRC13h (SEQ ID NO: 12), VRC14 (SEQ ID NO: 13), VRC14b (SEQ ID NO: 14), VRC14c (SEQ ID NO: 15), and VRC15 (SEQ ID NO: 16) antibodies, and the light chains of the VRC13 (SEQ ID NO: 17), VRC13b (SEQ ID NO: 18), VRC13c (SEQ ID NO: 19), VRC13d (SEQ ID NO: 20), VRC13e (SEQ ID NO: 21), VRC13f (SEQ ID NO: 22), VRC13g (SEQ ID NO: 23), VRC13h (SEQ ID NO: 24), VRC14 (SEQ ID NO: 25), VRC14b (SEQ ID NO: 26), VRC14c (SEQ ID NO: 27), and VRC15 (SEQ ID NO: 28) antibodies are shown. IGHV1-69*01 (SEQ ID NO: 69) and IGHJ2*01 (SEQ ID NO: 70) germline sequences are shown in FIG. 21A and IGLV2-14*01 (SEQ ID NO: 71) and IGLJ1*01 (SEQ ID NO: 72) germline sequences are shown in FIG. 21B. The IMGT CDR and framework sequences are underlined. All 12 antibodies are the result of the same B-cell recombination event (V-D-J recombination) and therefore are somatic variants of each other. Germline sequences are highlighted in bold and residues un-mutated from germline are indicated by a period (‘.’). All twelve antibodies are highly mutated from germline.

FIGS. 22A and 22B are a set of graphs showing that the 12 VRC13 mAbs bind to the RSC3 probe but not CD4bs knockout protein RSC3 Δ371I\P363N, indicating CD4bs specificity.

FIG. 23 is a graph. The data shows that mAbs VRC13, VRC14, VRC14b, VRC14c and VRC15 cross-compete (block) binding of CD4 to the YU2 gp120 HIV Env protein. These data confirm that these antibodies are directed to the CD4bs.

FIGS. 24A-24G are tables showing neutralization data for VRC16 and related clones. FIG. 24A provides a table showing IC50 and IC80 values for VRC16 neutralization of exemplary HIV-1 strains.

FIGS. 24B-24D are tables showing IC50 values for VRC16 neutralization of an expanded set of exemplary HIV-1 strains. FIGS. 24E-24G are tables showing IC80 values for VRC16 neutralization of an expanded set of exemplary HIV-1 strains. IC50 and IC80 values shown are in μg/ml.

FIGS. 25A-25G tables showing neutralization data for VRC13 and related clones. FIG. 25A is a table showing IC50 and IC80 values for VRC13 neutralization of exemplary HIV-1 strains. FIGS. 25B-25D are tables showing IC50 values for VRC13 neutralization of an expanded set of exemplary HIV-1 strains. FIGS. 25E-25G are tables showing IC80 values for VRC13 neutralization of an expanded set of exemplary HIV-1 strains. IC50 and IC80 values shown are in μg/ml.

FIG. 26 is a table showing results an immunoglobulin gene analysis of the VRC13 CD4-binding site neutralizing mAbs.

FIGS. 27A and 27B show the amino acid sequences of the heavy chains of the VRC13 (SEQ ID NO: 5), VRC13b (SEQ ID NO: 6), VRC13c (SEQ ID NO: 7), VRC13d (SEQ ID NO: 8), VRC13e (SEQ ID NO: 9), VRC13f (SEQ ID NO: 10), VRC13g (SEQ ID NO: 11), VRC13h (SEQ ID NO: 12), VRC14 (SEQ ID NO: 13), VRC14b (SEQ ID NO: 14), VRC14c (SEQ ID NO: 15), and VRC15 (SEQ ID NO: 16) antibodies, and the light chains of the VRC13 (SEQ ID NO: 17), VRC13b (SEQ ID NO: 18), VRC13c (SEQ ID NO: 19), VRC13d (SEQ ID NO: 20), VRC13e (SEQ ID NO: 21), VRC13f (SEQ ID NO: 22), VRC13g (SEQ ID NO: 23), VRC13h (SEQ ID NO: 24), VRC14 (SEQ ID NO: 25), VRC14b (SEQ ID NO: 26), VRC14c (SEQ ID NO: 27), and VRC15 (SEQ ID NO: 28) antibodies. The IGHV1-69*01 (SEQ ID NO: 69) and IGLV2-14*01 (SEQ ID NO: 70) germline sequences are also shown. The Kabat CDR sequences are underlined.

FIGS. 28A and 28B show the amino acid sequences of the heavy chains of the VRC13 (SEQ ID NO: 5), VRC13b (SEQ ID NO: 6), VRC13c (SEQ ID NO: 7), VRC13d (SEQ ID NO: 8), VRC13e (SEQ ID NO: 9), VRC13f (SEQ ID NO: 10), VRC13g (SEQ ID NO: 11), VRC13h (SEQ ID NO: 12), VRC14 (SEQ ID NO: 13), VRC14b (SEQ ID NO: 14), VRC14c (SEQ ID NO: 15), and VRC15 (SEQ ID NO: 16) antibodies, and the light chains of the VRC13 (SEQ ID NO: 17), VRC13b (SEQ ID NO: 18), VRC13c (SEQ ID NO: 19), VRC13d (SEQ ID NO: 20), VRC13e (SEQ ID NO: 21), VRC13f (SEQ ID NO: 22), VRC13g (SEQ ID NO: 23), VRC13h (SEQ ID NO: 24), VRC14 (SEQ ID NO: 25), VRC14b (SEQ ID NO: 26), VRC14c (SEQ ID NO: 27), and VRC15 (SEQ ID NO: 28) antibodies. The IGHV1-69*01 (SEQ ID NO: 69) and IGLV2-14*01 (SEQ ID NO: 70) germline sequences are also shown. The IMGT CDR sequences are underlined.

FIGS. 29A and 29B show the amino acid sequences of the heavy chains (FIG. 29A) of the VRC16 (SEQ ID NO: 29), VRC16b (SEQ ID NO: 30), VRC16c (SEQ ID NO: 31), and VRC16d (SEQ ID NO: 32) antibodies, and the light chains (FIG. 29B) of the VRC16 (SEQ ID NO: 33), VRC16b (SEQ ID NO: 34), VRC16c (SEQ ID NO: 35), and VRC16d (SEQ ID NO: 36) antibodies. The IGHV3-23*01 (SEQ ID NO: 73) and IGKV1-39*01 (SEQ ID NO: 74) germline sequences are shown in FIGS. 29A and 29B, respectively. The Kabat CDR sequences are underlined.

FIG. 30 shows the amino acid sequences of the heavy chains of the VRC16 (SEQ ID NO: 29), VRC16b (SEQ ID NO: 30), VRC16c (SEQ ID NO: 31), and VRC16d (SEQ ID NO: 32) antibodies, and the light chains of the VRC16 (SEQ ID NO: 33), VRC16b (SEQ ID NO: 34), VRC16c (SEQ ID NO: 35), and VRC16d (SEQ ID NO: 36) antibodies. The IGHV3-23*01 (SEQ ID NO: 71) and IGKV1-39*01 (SEQ ID NO: 72) germline sequences are also shown. The IMGT CDR sequences are underlined.

FIGS. 31A-31C are a set of tables showing the interactions between VRC13 and gp120 based on the co-crystal structure of VRC13 and gp120. The tables in FIGS. 31A-31B list gp120 epitope contacts, and the tables in FIGS. 31C list VRC13 paratope contacts. The VRC13 light chain does not contact gp120. “H” indicates a hydrogen bond, “S” indicates a salt bridge, and “HS” indicates a hydrogen bond and a salt bridge.

FIGS. 32A-32C are tables showing the interactions between VRC16 and gp120 based on the co-crystal structure of VRC16 and gp120. The tables in FIG. 32A list VRC16 heavy chain residues that contact gp120. FIG. 32B lists gp120 residues that contact VRC16 heavy chain. FIG. 32C lists VRC16 light chain residues that contact gp120, and gp120 residues that contact VRC16 light chain. “H” indicates a hydrogen bond, “S” indicates a salt bridge, and “HS” indicates a hydrogen bond and a salt bridge. The tables in FIG. 32A list gp120 epitope contacts, and the tables in FIGS. 31C list VRC13 paratope contacts.

FIGS. 33A-33C are a set of graphs and diagrams illustrating that broadly neutralizing CD4-binding-site antibodies can derive from diverse V_{H}-genes. (A) A maximum likelihood tree illustrates VH germline family relationships. Previously isolated neutralizing CD4bs antibodies that are VH1-2 are included in tree, and VRC13, VRC16 and HJ16 are indicated. The most common VH germline genes VH families are indicated. (B) Competition ELISAs were performed with a single concentration of biotin-labeled VRC13 and VRC16. Unlabeled mAbs were titrated into the ELISA at increasing concentrations to evaluate the effect on mAb binding to Yu2 gp120 (VRC13 & VRC16). (C) Dendograms display neutralization by VRC13 and VRC16 against 178 genetically diversified HIV-1 Env-pseudoviruses representing the major circulating clades. Neighbor joining trees display the protein distance of gp160 sequences from 178 gp160 sequences. A scale bar denotes 2% distance in amino acid sequence. Tree branches are colored by the neutralization potencies of VRC13 and VRC16 against each virus.

FIGS. 34A-34D are a set of diagrams and sequence alignments illustrating the recognition of the gp120 CD4bs by VRC13 and VRC16. (A) Structures of VRC13 and VRC16 bound to gp120 are shown at atomic-level detail with polypeptide chains in ribbon representations. (B) The epitope of antibodies VRC13, VRC16 and VRC01 are shown on the molecular surface of gp120. (C) Amino acid sequences of heavy chains for VRC13 (SEQ ID NOs: 5 and 17, respectively) and VRC16 (SEQ ID NOs: 29 and 33, respectively) are aligned to their putative germline V and J genes. VRC13 heavy chain is aligned with IGHV1-69*01 (SEQ ID NO: 69) and IGHJ2*01 (SEQ ID NO: 70). VRC16 heavy chain is aligned with IGHV3-23*01 (SEQ ID NO: 73) and IGHJ4*02 (SEQ ID NO: 75). (D) Amino acid sequences of light chains for VRC13 (SEQ ID NOs: 5 and 17, respectively) and VRC16 (SEQ ID NOs: 29 and 33, respectively) are aligned to their putative germline V and J genes. VRC13 light chain is aligned with IGLV2-14*01 (SEQ ID NO: 71) and IGLJ1*01 (SEQ ID NO: 72). VRC16 light chain is aligned with IGKV1-39*01 (SEQ ID NO: 74) and IGKJ1*01 (SEQ ID NO: 76). Residues that remain germline are indicated by a dash, and alignments are organized by CDR and FR regions. Antibody residues that contact gp120 are highlighted with a star for side-chain interactions, an open circle for main chain contacts and a filled circle for contacts that are both main and side chain. The light chain of VRC13 does not contact gp120.

FIG. 35 is a set of graphs showing the gp120 binding of VRC13, VRC16, and VRC01 antibodies with V gene germline reversion mutations. The results show that the VRC13 and VRC16 germline revertants retain binding for gp120, whereas the VRC01 germline revertants do not. (A) Binding of mature, V-gene reverted (gH-gL) and chimeric (mH-gL, gH-mL) antibodies was tested against gp120s from five different clades of HIV. The antibodies were immobilized on a sensor chip for SPR kinetic binding analysis with the proteins shown. The dissociation constant for each antibody and protein pairing is indicated by a single dot.

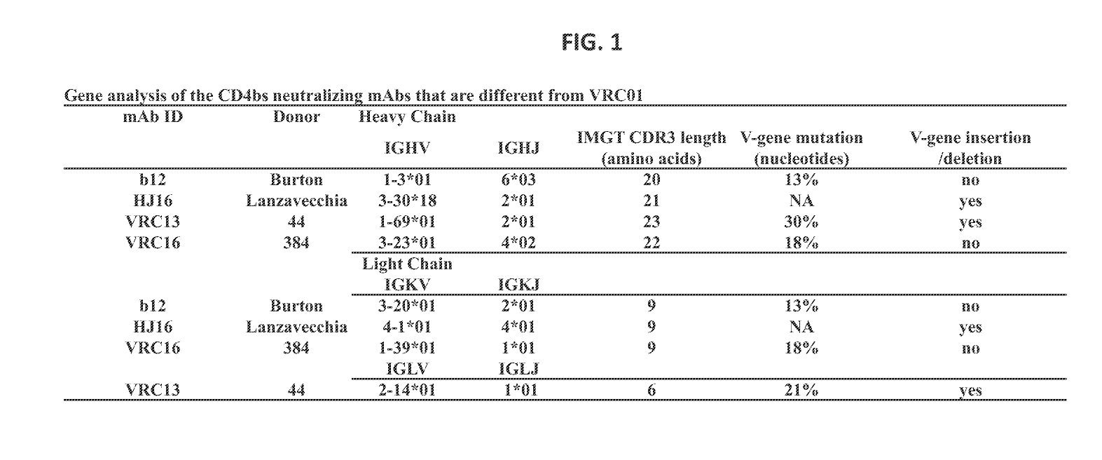

### SEQUENCE LISTING

The nucleic and amino acid sequences listed in the accompanying sequence listing are shown using standard letter abbreviations for nucleotide bases, and one or three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file in the form of the file named “Sequence.txt” 76 kb), which was created on Mar. 13, 2013, and is incorporated by reference herein. In the accompanying Sequence Listing:

SEQ ID NO: 1 is a VRC13 heavy chain consensus amino acid sequence.

SEQ ID NO: 2 is a VRC13 light chain consensus amino acid sequence.

SEQ ID NO: 3 is a VRC16 heavy chain consensus amino acid sequence.

SEQ ID NO: 4 is a VRC16 light chain consensus amino acid sequence.

SEQ ID NOs: 5-12 are the amino acid sequences of the heavy chain variable regions of the VRC13, VRC13b, VRC13c, VRC13d, VRC13e, VRC13f, VRC13g, and VRC13h antibodies, respectively.

SEQ ID NOs: 13-15 are the amino acid sequences of the heavy chain variable regions of the VRC14, VRC14b, and VRC14c antibodies, respectively.

SEQ ID NO: 16 is the amino acid sequence of the heavy chain variable region of the VRC15 antibody.

SEQ ID NOs: 17-24 are the amino acid sequences of the light chain variable regions of the VRC13, VRC13b, VRC13c, VRC13d, VRC13e, VRC13f, VRC13g, and VRC13h antibodies, respectively.

SEQ ID NOs: 25-27 are the amino acid sequences of the light chain variable regions of the VRC14, VRC14b, and VRC14c antibodies, respectively.

SEQ ID NO: 28 is the amino acid sequence of the light chain variable region of the VRC15 antibody.

SEQ ID NOs: 29-32 are the amino acid sequences of the heavy chain variable regions of the VRC16, VRC16b, VRC16c, and VRC16d antibodies, respectively.

SEQ ID NOs: 33-36 are the amino acid sequences of the light chain variable regions of the VRC16, VRC16b, VRC16c, and VRC16d antibodies, respectively.

SEQ ID NOs: 37-44 are exemplary nucleic acid sequences encoding the heavy chain variable regions of the VRC13, VRC13b, VRC13c, VRC13d, VRC13e, VRC13f, VRC13g, and VRC13h antibodies, respectively.

SEQ ID NOs: 45-47 are exemplary nucleic acid sequences encoding the heavy chain variable regions of the VRC14, VRC14b, and VRC14c antibodies, respectively.

SEQ ID NO: 48 is an exemplary nucleic acid sequence encoding the heavy chain variable region of the VRC15 antibody.

SEQ ID NOs: 49-56 are exemplary nucleic acid sequences encoding the light chain variable regions of the VRC13, VRC13b, VRC13c, VRC13d, VRC13e, VRC13f, VRC13g, and VRC13h antibodies, respectively.

SEQ ID NOs: 57-59 are exemplary nucleic acid sequences encoding the light chain variable regions of the VRC14, VRC14b, and VRC14c antibodies, respectively.

SEQ ID NO: 60 is an exemplary nucleic acid sequence encoding the light chain variable region of the VRC15 antibody.

SEQ ID NOs: 61-64 are exemplary nucleic acid sequences encoding the heavy chain variable regions of the VRC16, VRC16b, VRC16c, and VRC16d antibodies, respectively.

SEQ ID NOs: 65-68 are exemplary nucleic acid sequences encoding the light chain variable regions of the VRC16, VRC16b, VRC16c, and VRC16d antibodies, respectively.

SEQ ID NO: 69 is a IGHV1-69*01 germline amino acid sequence.

SEQ ID NO: 70 is a IGHJ2*01 germline amino acid sequence.

SEQ ID NO: 71 is a IGLV2-14 germline amino acid sequence.

SEQ ID NO: 72 is a IGLJ1*01 germline amino acid sequence.

SEQ ID NO: 73 is a IGHV3-23*01 germline amino acid sequence.

SEQ ID NO: 74 is a IGKV1-39*01 germline amino acid sequence.

SEQ ID NO: 75 is a IGHJ4*02 germline amino acid sequence.

SEQ ID NO: 76 is a IGKJ1*01 germline amino acid sequence.

SEQ ID NO: 77 is the amino acid sequence of a VRC13 heavy chain germline revertant.

SEQ ID NO: 78 is the amino acid sequence of a VRC13 light chain germline revertant.

SEQ ID NO: 79 is the amino acid sequence of a VRC16 heavy chain germline revertant.

SEQ ID NO: 80 is the amino acid sequence of a VRC16 light chain germline revertant.

### DETAILED DESCRIPTION

Isolated VRC13 and VRC16 monoclonal antibodies that specifically bind HIV-1 gp120 and are neutralizing, clonal variants of these monoclonal antibodies, compositions including these antibodies, nucleic acids encoding these antibodies, expression vectors comprising the nucleic acids, and isolated host cells that express the nucleic acids are disclosed herein. Clonal variants of these VRC13 and VRC16 antibodies are also provided that specifically bind gp120 and are neutralizing. These clonal variants are derived from specific germline sequences. Methods of using these antibodies are also provided.

### II. SUMMARY OF TERMS

Unless otherwise noted, technical terms are used according to conventional usage. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Terms describing protein structure and structural elements of proteins can be found in Creighton, Proteins, Structures and Molecular Properties, W.H. Freeman & Co., New York, 1993 (ISBN 0-717-7030) which is incorporated by reference herein in its entirety.

As used herein, the term “comprises” means “includes.” Thus, “comprising an antigen” means “including an antigen” without excluding other elements.

It is further to be understood that all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting.

To facilitate review of the various embodiments of this disclosure, the following explanations of terms are provided:

Administration: The introduction of a composition into a subject by a chosen route. Administration can be local or systemic. For example, if the chosen route is intravenous, the composition is administered by introducing the composition into a vein of the subject. In some examples a disclosed antibody specific for an HIV gp120 protein or polypeptide, or a nucleic acid encoding the antibody, is administered to a subject.

Agent: Any substance or any combination of substances that is useful for achieving an end or result; for example, a substance or combination of substances useful for inhibiting HIV infection in a subject. Agents include proteins, antibodies, nucleic acid molecules, compounds, small molecules, organic compounds, inorganic compounds, or other molecules of interest. An agent can include a therapeutic agent (such as an anti-retroviral agent), a diagnostic agent or a pharmaceutical agent. In some embodiments, the agent is a polypeptide agent (such as a HIV-neutralizing antibody), or an anti-viral agent. The skilled artisan will understand that particular agents may be useful to achieve more than one result.

Amino acid substitution: The replacement of one amino acid in peptide with a different amino acid.

Amplification: A technique that increases the number of copies of a nucleic acid molecule (such as an RNA or DNA). An example of amplification is the polymerase chain reaction, in which a biological sample is contacted with a pair of oligonucleotide primers, under conditions that allow for the hybridization of the primers to a nucleic acid template in the sample. The primers are extended under suitable conditions, dissociated from the template, and then re-annealed, extended, and dissociated to amplify the number of copies of the nucleic acid. The product of amplification can be characterized by electrophoresis, restriction endonuclease cleavage patterns, oligonucleotide hybridization or ligation, and/or nucleic acid sequencing using standard techniques. Other examples of amplification include strand displacement amplification, as disclosed in U.S. Pat. No. 5,744,311; transcription-free isothermal amplification, as disclosed in U.S. Pat. No. 6,033,881; repair chain reaction amplification, as disclosed in PCT Publication No. WO 90/01069; ligase chain reaction amplification, as disclosed in European Publication No. EP-A-320 308; gap filling ligase chain reaction amplification, as disclosed in U.S. Pat. No. 5,427,930; and NASBA™ RNA transcription-free amplification, as disclosed in U.S. Pat. No. 6,025,134.

Animal: A living multicellular vertebrate organism, a category that includes, for example, mammals and birds. A “mammal” includes both human and non-human mammals, such as mice. The term “subject” includes both human and animal subjects, such as non-human primates.

Antibody: A polypeptide substantially encoded by an immunoglobulin gene or immunoglobulin genes, or antigen binding fragments thereof, which specifically binds and recognizes an analyte (antigen) such as gp120 or an antigenic fragment of gp120 Immunoglobulin genes include the kappa, lambda, alpha, gamma, delta, epsilon and mu constant region genes, as well as the myriad immunoglobulin variable domain genes.

Antibodies exist, for example as intact immunoglobulins and as antigen binding fragments produced by digestion with various peptidases. For instance, Fabs, Fvs, and single-chain Fvs (scFvs) that specifically bind to gp120 or fragments of gp120 (that include the epitope bound by the originating antibody) would be gp120-specific binding agents. A scFv protein is a fusion protein in which a light chain variable domain of an immunoglobulin and a heavy chain variable domain of an immunoglobulin are bound by a linker, while in dsFvs, the chains have been mutated to introduce a disulfide bond to stabilize the association of the chains. The term also includes genetically engineered forms such as chimeric antibodies (such as humanized murine antibodies), heteroconjugate antibodies such as bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

Examples of antigen-binding antibody fragments include: (1) Fab, the fragment which contains a monovalent antigen-binding fragment of an antibody molecule produced by digestion of whole antibody with the enzyme papain to yield an intact light chain and a portion of one heavy chain; (2) Fab′, the fragment of an antibody molecule obtained by treating whole antibody with pepsin, followed by reduction, to yield an intact light chain and a portion of the heavy chain; two Fab′ fragments are obtained per antibody molecule; (3) (Fab′)₂, the fragment of the antibody obtained by treating whole antibody with the enzyme pepsin without subsequent reduction; (4) F(ab′)₂, a dimer of two Fab′ fragments held together by two disulfide bonds; (5) Fv, a genetically engineered fragment containing the variable domain of the light chain and the variable domain of the heavy chain expressed as two chains; and (6) single chain antibody (“SCA”), a genetically engineered molecule containing the variable domain of the light chain, the variable domain of the heavy chain, linked by a suitable polypeptide linker as a genetically fused single chain molecule. The term “antibody,” as used herein, also includes antibody fragments either produced by the modification of whole antibodies or those synthesized de novo using recombinant DNA methodologies.

Typically, a naturally occurring immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE.

Each heavy and light chain contains a constant region and a variable region, (the regions are also known as “domains”). In several embodiments, the heavy and the light chain variable domains combine to specifically bind the antigen. In additional embodiments, only the heavy chain variable domain is required. For example, naturally occurring camelid antibodies consisting of a heavy chain only are functional and stable in the absence of light chain (see, e.g., Hamers-Casterman et al., Nature, 363:446-448, 1993; Sheriff et al., Nat. Struct. Biol., 3:733-736, 1996). Light and heavy chain variable domains contain a “framework” region interrupted by three hypervariable regions, also called “complementarity-determining regions” or “CDRs” (see, e.g., Kabat et al., Sequences of Proteins of Immunological Interest, U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The amino acid sequence boundaries of a given CDR can be readily determined using any of a number of well-known schemes, including those described by Kabat et al. (“Sequences of Proteins of Immunological Interest,” 5th Ed. Public Health Service, National Institutes of Health, Bethesda, Md., 1991; “Kabat” numbering scheme), Al-Lazikani et al., (JMB 273,927-948, 1997; “Chothia” numbering scheme), and Lefranc et al. (“IMGT unique numbering for immunoglobulin and T cell receptor variable domains and Ig superfamily V-like domains,” Dev. Comp. Immunol., 27:55-77, 2003; “IMGT” numbering scheme).

The CDRs of each chain are typically referred to as CDR1, CDR2, and CDR3 (from the N-terminus to C-terminus), and are also typically identified by the chain in which the particular CDR is located. Thus, a V_{H} CDR3 is the CDR3 from the variable domain of the heavy chain of the antibody in which it is found, whereas a V_{L} CDR1 is the CDR1 from the variable domain of the light chain of the antibody in which it is found. Light chain CDRs are sometimes referred to as LCDR1, LCDR2, and LCDR3. Heavy chain CDRs are sometimes referred to as LCDR1, LCDR2, and LCDR3.

References to “V_{H}” or “VH” refer to the variable domain of an immunoglobulin heavy chain, including that of an antibody fragment, such as Fv, scFv, dsFv or Fab. References to “V_{L}” or “VL” refer to the variable domain of an immunoglobulin light chain, including that of an Fv, scFv, dsFv or Fab.

A “monoclonal antibody” may be produced by a single clone of B-lymphocytes or by a cell including one or more nucleic acid molecules encoding the light and heavy chains of a single antibody have been transfected. Monoclonal antibodies are produced by methods known to those of skill in the art, for instance by making hybrid antibody-forming cells from a fusion of myeloma cells with immune spleen cells. These fused cells and their progeny are termed “hybridomas.” Monoclonal antibodies include humanized and fully human monoclonal antibodies. In some examples monoclonal antibodies are isolated from a subject. The amino acid sequences of such isolated monoclonal antibodies can be determined.

A “humanized” immunoglobulin is an immunoglobulin including a human framework region and one or more CDRs from a non-human (such as a mouse, rat, or synthetic) immunoglobulin. The non-human immunoglobulin providing the CDRs is termed a “donor,” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, such as at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody including a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions, such as in the framework region, which have substantially no effect on antigen binding or other immunoglobulin functions. Humanized immunoglobulins can be constructed by means of genetic engineering (for example, see U.S. Pat. No. 5,585,089).

Antibody Immunogenicity: A property of an antibody, whereby the antibody generates an immune response when administered to a subject, such as a human subject. In several embodiments, a disclosed antibody is not immunogenic or has low immunogenicity, for example, a disclosed antibody is not significantly more immunogenic compared to a standard control, or a reference antibody. Methods of determining the immunogenicity of an antibody are known to the person of ordinary skill in the art (see, e.g., Krieckaert et al., Current Opin Rheumatol., 24:306-311, 2012; Stas and Lasters, IDrugs, 12:169-173, 2009). In one non-limiting example, immunogenicity can be determined by assaying plasma or serum from a test subject using an ELISA against the antibody of interest.

Antibody Scaffold: Refers to a heterologous protein that is engrafted with one or more CDRs from an antibody of interest on its surface. Transplantation of the CDRs can be performed computationally in a manner that preserves its relevant structure and conformation. Mutations within the acceptor scaffold are made in order to accommodate the CDR graft.

Antibody self-reactivity or autoreactivity: A property of an antibody, whereby the antibody reacts with self-epitopes, that is epitopes of proteins and/or lipids that are produced by the subject. An antibody that does not have self-reactivity does not substantially bind to epitopes or lipids present on the membrane of a cell from a subject. Methods of determining if an antibody reacts with self epitopes are known to the person of ordinary skill in the art and described herein (for example, in Examples 1 and 8). In one example, antibody self reactivity is evaluated using an anti-cardiolipin assay or an anti-nuclear antigen (ANA) assay. The anti-ANA assay can include an anti-ANA LUMINEX® assay or an ANA cell-staining assay, for example. In several embodiments, a disclosed antibody is not self-reactive (or autoreactive), or is minimally self-reactive. In one non-limiting example, a disclosed antibody is not significantly more self-reactive compared to the VRC01 antibody, for example as measured using an anti-ANA LUMINEX® assay or an ANA cell-staining assay. In another non-limiting example, a disclosed antibody noes not have self reactivity above background levels, for example, as measured using an anti-ANA LUMINEX® assay or an ANA cell-staining assay.

Antigen: A polypeptide that can stimulate the production of antibodies or a T cell response in an animal, including polypeptides that are injected or absorbed into an animal. An antigen reacts with the products of specific humoral or cellular immunity, including those induced by heterologous antigens, such as the disclosed antigens. “Epitope” or “antigenic determinant” refers to the region of an antigen to which B and/or T cells respond. In one embodiment, T cells respond to the epitope, when the epitope is presented in conjunction with an MHC molecule. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation. Methods of determining spatial conformation of epitopes include, for example, x-ray crystallography and nuclear magnetic resonance.

Immunogenic polypeptides and immunogenic peptides are non-limiting examples of antigens. In some examples, antigens include polypeptides derived from a pathogen of interest, such as a virus. An antigen that can stimulate the production of antibodies or a T cell response in a subject to a polypeptide expressed by a virus is a viral antigen. An “HIV antigen” can stimulate the production of antibodies or a T cell response in a subject to a polypeptide expressed by HIV. In some embodiments, an HIV antigen is a polypeptide expressed by HIV, such as HIV ENV, or a fragment thereof, such as gp120.

A “target epitope” is a specific epitope on an antigen that specifically binds an antibody of interest, such as a monoclonal antibody. In some examples, a target epitope includes the amino acid residues that contact the antibody of interest, such that the target epitope can be selected by the amino acid residues determined to be in contact with the antibody of interest.

Anti-retroviral agent: An agent that specifically inhibits a retrovirus from replicating or infecting cells. Non-limiting examples of antiretroviral drugs include entry inhibitors (e.g., enfuvirtide), CCR5 receptor antagonists (e.g., aplaviroc, vicriviroc, maraviroc), reverse transcriptase inhibitors (e.g., lamivudine, zidovudine, abacavir, tenofovir, emtricitabine, efavirenz), protease inhibitors (e.g., lopivar, ritonavir, raltegravir, darunavir, atazanavir), maturation inhibitors (e.g., alpha interferon, bevirimat and vivecon).

Anti-retroviral therapy (ART): A therapeutic treatment for HIV infection involving administration of at least one anti-retroviral agents (e.g., one, two, three or four anti-retroviral agents) to an HIV infected individual during a course of treatment. Non-limiting examples of antiretroviral agents include entry inhibitors (e.g., enfuvirtide), CCR5 receptor antagonists (e.g., aplaviroc, vicriviroc, maraviroc), reverse transcriptase inhibitors (e.g., lamivudine, zidovudine, abacavir, tenofovir, emtricitabine, efavirenz), protease inhibitors (e.g., lopivar, ritonavir, raltegravir, darunavir, atazanavir), maturation inhibitors (e.g., alpha interferon, bevirimat and vivecon). One example of an ART regimen includes treatment with a combination of tenofovir, emtricitabine and efavirenz. In some examples, ART includes Highly Active Anti-Retroviral Therapy (HAART).

Antigenic surface: A surface of a molecule, for example a protein such as a gp120 protein or polypeptide, capable of eliciting an immune response. An antigenic surface includes the defining features of that surface, for example the three-dimensional shape and the surface charge. An antigenic surface includes both surfaces that occur on gp120 polypeptides as well as surfaces of compounds that mimic the surface of a gp120 polypeptide (mimetics). In some examples, an antigenic surface includes all or part of the surface of gp120 that binds to the CD4 receptor.

Atomic Coordinates or Structure coordinates: Mathematical coordinates derived from mathematical equations related to the patterns obtained on diffraction of a monochromatic beam of X-rays by the atoms (scattering centers) such as an antigen, or an antigen in complex with an antibody. In some examples that antigen can be gp120, a gp120:antibody complex, or combinations thereof in a crystal. The diffraction data are used to calculate an electron density map of the repeating unit of the crystal. The electron density maps are used to establish the positions of the individual atoms within the unit cell of the crystal. In one example, the term “structure coordinates” refers to Cartesian coordinates derived from mathematical equations related to the patterns obtained on diffraction of a monochromatic beam of X-rays, such as by the atoms of a gp120 in crystal form.

Those of ordinary skill in the art understand that a set of structure coordinates determined by X-ray crystallography is not without standard error. For the purpose of this disclosure, any set of structure coordinates that have a root mean square deviation of protein backbone atoms (N, Cα, C and 0) of less than about 1.0 Angstroms when superimposed, such as about 0.75, or about 0.5, or about 0.25 Angstroms, using backbone atoms, shall (in the absence of an explicit statement to the contrary) be considered identical.

Those of skill in the art will understand that a set of structure coordinates, for example, for a disclosed antibody or portions thereof in complex with gp120 or a portion thereof, is a relative set of points that define a shape in three dimensions. Thus, it is possible that an entirely different set of coordinates could define a similar or identical shape. Moreover, slight variations in the individual coordinates will have little effect on overall shape. The variations in coordinates discussed above may be generated because of mathematical manipulations of the structure coordinates.

Binding affinity: Affinity of an antibody or antigen binding fragment thereof for an antigen. In one embodiment, affinity is calculated by a modification of the Scatchard method described by Frankel et al., Mol. Immunol., 16:101-106, 1979. In another embodiment, binding affinity is measured by an antigen/antibody dissociation rate. In yet another embodiment, a high binding affinity is measured by a competition radioimmunoassay. In several examples, a high binding affinity is at least about 1×10⁻⁸ M. In other embodiments, a high binding affinity is at least about 1.0×10⁻⁸, at least about 5.0×10⁻⁸, at least about 1.0×10⁻⁹, at least about 1.5×10⁻⁹, at least about 2.0×10⁻⁹, at least about 2.5×10⁻⁹, or at least about 3.0×10⁻⁹.

Bispecific antibody: A recombinant molecule composed of two different antigen binding domains that consequently bind to two different antigenic epitopes. Bispecific antibodies include chemically or genetically linked molecules of two antigen-binding domains. The antigen binding domains can be linked using a linker. The antigen binding domains can be monoclonal antibodies, antigen-binding fragments (e.g., Fab, scFv), eAds, bispecific single chain antibodies or combinations thereof. A bispecific antibody can include one or more constant domains, but does not necessarily include a constant domain. An example of a bispecific antibody is a bispecific single chain antibody including a scFv that specifically binds to gp120 joined (via a peptide linker) to a scFv that specifically binds to an antigen other than gp120. Another example is a bispecific antibody including a Fab that specifically binds to gp120 joined to a scFv that specifically binds to an antigen other than gp120.

CD4: Cluster of differentiation factor 4 polypeptide; a T-cell surface protein that mediates interaction with the MHC class II molecule. CD4 also serves as the primary receptor site for HIV on T-cells during HIV-I infection. CD4 is known to bind to gp120 from HIV. The known sequence of the CD4 precursor has a hydrophobic signal peptide, an extracellular region of approximately 370 amino acids, a highly hydrophobic stretch with significant identity to the membrane-spanning domain of the class II MHC beta chain, and a highly charged intracellular sequence of 40 resides (Maddon, Cell 42:93, 1985).

The term “CD4” includes polypeptide molecules that are derived from CD4 include fragments of CD4, generated either by chemical (for example enzymatic) digestion or genetic engineering means. Such a fragment may be one or more entire CD4 protein domains. The extracellular domain of CD4 consists of four contiguous immunoglobulin-like regions (D1, D2, D3, and D4, see Sakihama et al., Proc. Natl. Acad. Sci. 92:6444, 1995; U.S. Pat. No. 6,117,655), and amino acids 1 to 183 have been shown to be involved in gp120 binding. For instance, a binding molecule or binding domain derived from CD4 would comprise a sufficient portion of the CD4 protein to mediate specific and functional interaction between the binding fragment and a native or viral binding site of CD4. One such binding fragment includes both the D1 and D2 extracellular domains of CD4 (D1D2 is also a fragment of soluble CD4 or sCD4 which is comprised of D1 D2 D3 and D4), although smaller fragments may also provide specific and functional CD4-like binding. The gp120-binding site has been mapped to D1 of CD4.

CD4 polypeptides also include “CD4-derived molecules” which encompasses analogs (non-protein organic molecules), derivatives (chemically functionalized protein molecules obtained starting with the disclosed protein sequences) or mimetics (three-dimensionally similar chemicals) of the native CD4 structure, as well as proteins sequence variants or genetic alleles that maintain the ability to functionally bind to a target molecule.

CD4 binding site (CD4BS) antibodies: Antibodies that bind to or substantially overlap the CD4 binding surface of a gp120 polypeptide. The antibodies interfere with or prevent CD4 from binding to a gp120 polypeptide.

Chimeric antibody: An antibody which includes sequences derived from two different antibodies, which typically are of different species. In some examples, a chimeric antibody includes one or more CDRs and/or framework regions from one human antibody and CDRs and/or framework regions from another human antibody.

Clonal variant: Any sequence, which differs by one or more nucleotides or amino acids, in presence of V region with identical mutations compared to the germline, identical VDJ or VJ gene usage, and identical D and J length. The “germline” sequence is intended to be the sequence coding for the antibody/immunoglobulin (or of any fragment thereof) deprived of mutations, for example somatic mutations. The percentage of homology represents an indication of the mutational events which any type of heavy chain portion undergoes after contact with an antigen.

Contacting: Placement in direct physical association; includes both in solid and liquid form, which can take place either in vivo or in vitro. Contacting includes contact between one molecule and another molecule, for example the amino acid on the surface of one polypeptide, such as an antigen, that contacts another polypeptide, such as an antibody. Contacting can also include contacting a cell for example by placing an antibody in direct physical association with a cell.

Control: A reference standard. In some embodiments, the control is a negative control, such as sample obtained from a healthy patient not infected with HIV. In other embodiments, the control is a positive control, such as a tissue sample obtained from a patient diagnosed with HIV infection. In still other embodiments, the control is a historical control or standard reference value or range of values (such as a previously tested control sample, such as a group of HIV patients with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, or at least about 500%.

Detecting: To identify the existence, presence, or fact of something. General methods of detecting are known to the skilled artisan and may be supplemented with the protocols and reagents disclosed herein. For example, included herein are methods of detecting a cell that expresses gp120 in a subject.

DNA sequencing: The process of determining the nucleotide order of a given DNA molecule. The general characteristics of “deep sequencing” are that genetic material is amplified, such as by polymerase chain reaction, and then the amplified products are ligated to a solid surface. The sequence of the amplified target genetic material is then performed in parallel and the sequence information is captured by a computer. Generally, the sequencing can be performed using automated Sanger sequencing (AB13730x1 genome analyzer), pyrosequencing on a solid support (454 sequencing, Roche), sequencing-by-synthesis with reversible terminations (ILLUMINA® Genome Analyzer), sequencing-by-ligation (ABI SOLiD®) or sequencing-by-synthesis with virtual terminators (HELISCOPE®).

In some embodiments, DNA sequencing is performed using a chain termination method developed by Frederick Sanger, and thus termed “Sanger based sequencing” or “SBS.” This technique uses sequence-specific termination of a DNA synthesis reaction using modified nucleotide substrates. Extension is initiated at a specific site on the template DNA by using a short oligonucleotide primer complementary to the template at that region. The oligonucleotide primer is extended using DNA polymerase in the presence of the four deoxynucleotide bases (DNA building blocks), along with a low concentration of a chain terminating nucleotide (most commonly a di-deoxynucleotide). Limited incorporation of the chain terminating nucleotide by the DNA polymerase results in a series of related DNA fragments that are terminated only at positions where that particular nucleotide is present. The fragments are then size-separated by electrophoresis a polyacrylamide gel, or in a narrow glass tube (capillary) filled with a viscous polymer. An alternative to using a labeled primer is to use labeled terminators instead; this method is commonly called “dye terminator sequencing.”

“Pyrosequencing” is an array based method, which has been commercialized by 454 Life Sciences. In some embodiments of the array-based methods, single-stranded DNA is annealed to beads and amplified via EmPCR®. These DNA-bound beads are then placed into wells on a fiber-optic chip along with enzymes that produce light in the presence of ATP. When free nucleotides are washed over this chip, light is produced as the PCR amplification occurs and ATP is generated when nucleotides join with their complementary base pairs. Addition of one (or more) nucleotide(s) results in a reaction that generates a light signal that is recorded, such as by the charge coupled device (CCD) camera, within the instrument. The signal strength is proportional to the number of nucleotides, for example, homopolymer stretches, incorporated in a single nucleotide flow.

Effector molecule: The portion of a chimeric molecule that is intended to have a desired effect on a cell to which the chimeric molecule is targeted. Effector molecule is also known as an effector moiety (EM), therapeutic agent, or diagnostic agent, or similar terms.

Epitope: An antigenic determinant. These are particular chemical groups or peptide sequences on a molecule that are antigenic, i.e. that elicit a specific immune response. An antibody specifically binds a particular antigenic epitope on a polypeptide. In some examples a disclosed antibody specifically binds to an epitope on the surface of gp120 from HIV.

Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation. Methods of determining spatial conformation of epitopes include, for example, x-ray crystallography and nuclear magnetic resonance. Epitopes can also include post-translation modification of amino acids, such as N-linked glycosylation.

Fc polypeptide: The polypeptide including the constant region of an antibody excluding the first constant region immunoglobulin domain. Fc region generally refers to the last two constant region immunoglobulin domains of IgA, IgD, and IgG, and the last three constant region immunoglobulin domains of IgE and IgM. An Fc region may also include part or all of the flexible hinge N-terminal to these domains. For IgA and IgM, an Fc region may or may not include the tailpiece, and may or may not be bound by the J chain. For IgG, the Fc region includes immunoglobulin domains Cgamma2 and Cgamma3 (Cγ2 and Cγ3) and the lower part of the hinge between Cgammal (Cγ1) and Cγ2. Although the boundaries of the Fc region may vary, the human IgG heavy chain Fc region is usually defined to include residues C226 or P230 to its carboxyl-terminus, wherein the numbering is according to the EU index as in Kabat. For IgA, the Fc region includes immunoglobulin domains Calpha2 and Calpha3 (Cα2 and Cα3) and the lower part of the hinge between Calpha1 (Cα1) and Cα2. Encompassed within the definition of the Fc region are functionally equivalent analogs and variants of the Fc region. A functionally equivalent analog of the Fc region may be a variant Fc region, including one or more amino acid modifications relative to the wild-type or naturally existing Fc region. Variant Fc regions will possess at least 50% homology with a naturally existing Fc region, such as about 80%, and about 90%, or at least about 95% homology. Functionally equivalent analogs of the Fc region may include one or more amino acid residues added to or deleted from the N- or C-termini of the protein, such as no more than 30 or no more than 10 additions and/or deletions. Functionally equivalent analogs of the Fc region include Fc regions operably linked to a fusion partner. Functionally equivalent analogs of the Fc region must include the majority of all of the Ig domains that compose Fc region as defined above; for example IgG and IgA Fc regions as defined herein must include the majority of the sequence encoding CH₂ and the majority of the sequence encoding CH₃. Thus, the CH₂ domain on its own, or the CH₃ domain on its own, are not considered Fc region. The Fc region may refer to this region in isolation, or this region in the context of an Fc fusion polypeptide (immunoadhesin, see below).

Framework Region: Amino acid sequences interposed between CDRs. Includes variable light and variable heavy framework regions. The framework regions serve to hold the CDRs in an appropriate orientation for antigen binding.

gp120: An envelope protein from Human Immunodeficiency Virus (HIV). This envelope protein is initially synthesized as a longer precursor protein of 845-870 amino acids in size, designated gp160. gp160 is cleaved by a cellular protease into gp120 and gp41. gp120 contains most of the external, surface-exposed, domains of the HIV envelope glycoprotein complex, and it is gp120 which binds both to cellular CD4 receptors and to cellular chemokine receptors (such as CCR5).

The mature gp120 wildtype polypeptides have about 500 amino acids in the primary sequence. gp120 is heavily N-glycosylated giving rise to an apparent molecular weight of 120 kD. The polypeptide is comprised of five conserved regions (C1-05) and five regions of high variability (V1-V5). Exemplary sequence of wt gp120 polypeptides are shown on GENBANK®, for example accession numbers AAB05604 and AAD12142 (as available on Oct. 16, 2009), incorporated by reference herein. It is understood that there are numerous variation in the sequence of gp120 from what is given in GENBANK®, for example accession numbers AAB05604 and AAD12142, and that these variants are skill recognized in the art as gp120.

Highly active anti-retroviral therapy (HAART): A therapeutic treatment for HIV infection involving administration of multiple anti-retroviral agents (e.g., two, three or four anti-retroviral agents) to an HIV infected individual during a course of treatment. Non-limiting examples of antiretroviral agents include entry inhibitors (e.g., enfuvirtide), CCR5 receptor antagonists (e.g., aplaviroc, vicriviroc, maraviroc), reverse transcriptase inhibitors (e.g., lamivudine, zidovudine, abacavir, tenofovir, emtricitabine, efavirenz), protease inhibitors (e.g., lopivar, ritonavir, raltegravir, darunavir, atazanavir), maturation inhibitors (e.g., alpha interferon, bevirimat and vivecon). One example of a HAART regimen includes treatment with a combination of tenofovir, emtricitabine and efavirenz.

HIV Envelope protein (Env): The HIV envelope protein is initially synthesized as a longer precursor protein of 845-870 amino acids in size, designated gp160. gp160 forms a homotrimer and undergoes glycosylation within the Golgi apparatus. In vivo, it is then cleaved by a cellular protease into gp120 and gp41. gp120 contains most of the external, surface-exposed, domains of the HIV envelope glycoprotein complex, and it is gp120 which binds both to cellular CD4 receptors and to cellular chemokine receptors (such as CCR5). gp41 contains a transmembrane domain and remains in a trimeric configuration; it interacts with gp120 in a non-covalent manner.

Host cells: Cells in which a vector can be propagated and its DNA expressed, for example a disclosed antibody can be expressed in a host cell. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Human Immunodeficiency Virus (HIV): A retrovirus that causes immunosuppression in humans (HIV disease), and leads to a disease complex known as the acquired immunodeficiency syndrome (AIDS). “HIV disease” refers to a well-recognized constellation of signs and symptoms (including the development of opportunistic infections) in persons who are infected by an HIV virus, as determined by antibody or western blot studies. Laboratory findings associated with this disease include a progressive decline in T cells. HIV includes HIV type 1 (HIV-1) and HIV type 2 (HIV-2). Related viruses that are used as animal models include simian immunodeficiency virus (SIV), and feline immunodeficiency virus (FIV). Treatment of HIV-1 with HAART has been effective in reducing the viral burden and ameliorating the effects of HIV-1 infection in infected individuals.

HXB2 numbering system: A reference numbering system for HIV protein and nucleic acid sequences, using HIV-1 HXB2 strain sequences as a reference for all other HIV strain sequences. The person of ordinary skill in the art is familiar with the HXB2 numbering system, and this system is set forth in “Numbering Positions in HIV Relative to HXB2CG,” Bette Korber et al., Human Retroviruses and AIDS 1998: A Compilation and Analysis of Nucleic Acid and Amino Acid Sequences. Korber B, Kuiken CL, Foley B, Hahn B, McCutchan F, Mellors J W, and Sodroski J, Eds. Theoretical Biology and Biophysics Group, Los Alamos National Laboratory, Los Alamos, N. Mex., which is incorporated by reference herein in its entirety. HXB2 is also known as: HXBc2, for HXB clone 2; HXB2R, in the Los Alamos HIV database, with the R for revised, as it was slightly revised relative to the original HXB2 sequence; and HXB2CG in GENBANK™, for HXB2 complete genome. The numbering used in gp120 polypeptides disclosed herein is relative to the HXB2 numbering scheme.

Immune complex: The binding of antibody to a soluble antigen forms an immune complex. The formation of an immune complex can be detected through conventional methods known to the skilled artisan, for instance immunohistochemistry, immunoprecipitation, flow cytometry, immunofluorescence microscopy, ELISA, immunoblotting (for example, Western blot), magnetic resonance imaging, CT scans, X-ray and affinity chromatography Immunological binding properties of selected antibodies may be quantified using methods well known in the art.

Immunoadhesin: A molecular fusion of a protein with the Fc region of an immunoglobulin, wherein the immunoglobulin retains specific properties, such as Fc receptor binding and increased half-life. An Fc fusion combines the Fc region of an immunoglobulin with a fusion partner, which in general can be any protein, polypeptide, peptide, or small molecule. In one example, and immunoadhesin includes the hinge, CH₂, and CH₃ domains of the immunoglobulin gamma 1 heavy chain constant region. In another example, the immunoadhesin includes the CH₂, and CH₃ domains of an IgG.

Immunological Probe: A molecule that can be used for selection of antibodies from sera which are directed against a specific epitope, including from human patient sera. The epitope scaffolds, along with related point mutants, can be used as immunological probes in both positive and negative selection of antibodies against the epitope graft. In some examples immunological probes are engineered variants of gp120.

Immunologically reactive conditions: Includes reference to conditions which allow an antibody raised against a particular epitope to bind to that epitope to a detectably greater degree than, and/or to the substantial exclusion of, binding to substantially all other epitopes Immunologically reactive conditions are dependent upon the format of the antibody binding reaction and typically are those utilized in immunoassay protocols or those conditions encountered in vivo. See Harlow & Lane, supra, for a description of immunoassay formats and conditions. The immunologically reactive conditions employed in the methods are “physiological conditions” which include reference to conditions (e.g., temperature, osmolarity, pH) that are typical inside a living mammal or a mammalian cell. While it is recognized that some organs are subject to extreme conditions, the intra-organismal and intracellular environment normally lies around pH 7 (e.g., from pH 6.0 to pH 8.0, more typically pH 6.5 to 7.5), contains water as the predominant solvent, and exists at a temperature above 0° C. and below 50° C. Osmolarity is within the range that is supportive of cell viability and proliferation.

IgA: A polypeptide belonging to the class of antibodies that are substantially encoded by a recognized immunoglobulin alpha gene. In humans, this class or isotype comprises IgA₁ and IgA₂. IgA antibodies can exist as monomers, polymers (referred to as pIgA) of predominantly dimeric form, and secretory IgA. The constant chain of wild-type IgA contains an 18-amino-acid extension at its C-terminus called the tail piece (tp). Polymeric IgA is secreted by plasma cells with a 15-kDa peptide called the J chain linking two monomers of IgA through the conserved cysteine residue in the tail piece.

IgG: A polypeptide belonging to the class or isotype of antibodies that are substantially encoded by a recognized immunoglobulin gamma gene. In humans, this class comprises IgG₁, IgG₂, IgG₃, and IgG₄. In mice, this class comprises IgG₁, IgG₂ₐ, IgG_{2b}, IgG₃

Inhibiting or treating a disease: Inhibiting the full development of a disease or condition, for example, in a subject who is at risk for a disease such as acquired immunodeficiency syndrome (AIDS). “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology.

Isolated: An “isolated” biological component (such as a cell, for example a B cell, a nucleic acid, peptide, protein or antibody) has been substantially separated, produced apart from, or purified away from other biological components in the cell of the organism in which the component naturally occurs, such as, other chromosomal and extrachromosomal DNA and RNA, and proteins. Nucleic acids, peptides and proteins which have been “isolated” thus include nucleic acids and proteins purified by standard purification methods. The term also embraces nucleic acids, peptides, and proteins prepared by recombinant expression in a host cell as well as chemically synthesized nucleic acids. In some examples an antibody, such as an antibody specific for gp120 can be isolated, for example isolated from a subject infected with HIV.

K_{d}: The dissociation constant for a given interaction, such as a polypeptide ligand interaction or an antibody antigen interaction. For example, for the bimolecular interaction of an antibody (such as VRC13 or VRC16 or a variant thereof as disclosed herein) and an antigen (such as gp120) it is the concentration of the individual components of the bimolecular interaction divided by the concentration of the complex.

Label: A detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. In some examples, a disclosed antibody as labeled.

Linker: A bi-functional molecule that can be used to link two molecules into one contiguous molecule, for example, to link an effector molecule to an antibody. In some embodiments, a conjugate includes a linker between the effector molecule or detectable marker and an antibody. In some embodiments, the linker is cleavable under intracellular conditions, such that cleavage of the linker releases the effector molecule or detectable marker from the antibody in the intracellular environment. In yet other embodiments, the linker is not cleavable and the effector molecule or detectable marker can be released, for example, by antibody degradation. In some cases, a linker is a peptide within an antigenbinding fragment (such as an Fv fragment) which serves to indirectly bond the variable heavy chain to the variable light chain.

In several embodiments, the terms “conjugating,” “joining,” “bonding” or “linking” refer to making two polypeptides into one contiguous polypeptide molecule, to covalently attaching a radionuclide or other molecule to a polypeptide, such as an antibody that specifically binds gp120, or an antigen binding fragment thereof. In the specific context, the terms include reference to joining a ligand, such as an antibody moiety, to an effector molecule. The linkage can be either by chemical or recombinant means. “Chemical means” refers to a reaction between the antibody moiety and the effector molecule such that there is a covalent bond formed between the two molecules to form one molecule.

Neutralizing antibody: An antibody which reduces the infectious titer of an infectious agent by binding to a specific antigen on the infectious agent. In some examples the infectious agent is a virus. In some examples, an antibody that is specific for gp120 neutralizes the infectious titer of HIV. A “broadly neutralizing antibody” is an antibody that binds to and inhibits the function of related antigens, such as antigens that share at least 85%, 90%, 95%, 96%, 97%, 98% or 99% identity antigenic surface of antigen. With regard to an antigen from a pathogen, such as a virus, the antibody can bind to and inhibit the function of an antigen from more than one class and/or subclass of the pathogen. For example, with regard to a human immunodeficiency virus, the antibody can bind to and inhibit the function of an antigen, such as gp120 from more than one Glade. In one embodiment, broadly neutralizing antibodies to HIV are distinct from other antibodies to HIV in that they neutralize a high percentage of the many types of HIV in circulation.

Nucleic acid: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Recombinant nucleic acid” refers to a nucleic acid having nucleotide sequences that are not naturally joined together. This includes nucleic acid vectors comprising an amplified or assembled nucleic acid which can be used to transform a suitable host cell. A host cell that comprises the recombinant nucleic acid is referred to as a “recombinant host cell.” The gene is then expressed in the recombinant host cell to produce, e.g., a “recombinant polypeptide.” A recombinant nucleic acid may serve a non-coding function (e.g., promoter, origin of replication, ribosome-binding site, etc.) as well.

A first sequence is an “antisense” with respect to a second sequence if a polynucleotide whose sequence is the first sequence specifically hybridizes with a polynucleotide whose sequence is the second sequence.

Terms used to describe sequence relationships between two or more nucleotide sequences or amino acid sequences include “reference sequence,” “selected from,” “comparison window,” “identical,” “percentage of sequence identity,” “substantially identical,” “complementary,” and “substantially complementary.”

For sequence comparison of nucleic acid sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well known in the art. Optimal alignment of sequences for comparison can be conducted, e.g., by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of Pearson & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds 1995 supplement)).

A polynucleotide or nucleic acid sequence refers to a polymeric form of nucleotide at least 10 bases in length. A recombinant polynucleotide includes a polynucleotide that is not immediately contiguous with both of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an autonomously replicating plasmid or virus; or into the genomic DNA of a prokaryote or eukaryote, or which exists as a separate molecule (e.g., a cDNA) independent of other sequences. The nucleotides can be ribonucleotides, deoxyribonucleotides, or modified forms of either nucleotide. The term includes single- and double-stranded forms of DNA. A gp120 polynucleotide is a nucleic acid encoding a gp120 polypeptide.

Operably linked: A first nucleic acid sequence is operably linked with a second nucleic acid sequence when the first nucleic acid sequence is placed in a functional relationship with the second nucleic acid sequence. For instance, a promoter, such as the CMV promoter, is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carriers: The pharmaceutically acceptable carriers of use are conventional. Remington's Pharmaceutical Sciences, by E. W. Martin, Mack Publishing Co., Easton, Pa., 19th Edition, 1995, describes compositions and formulations suitable for pharmaceutical delivery of the disclosed antibodies.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Pharmaceutical agent: A chemical compound or composition capable of inducing a desired therapeutic or prophylactic effect when properly administered to a subject or a cell. In some examples a pharmaceutical agent includes one or more of the disclosed antibodies.

Polypeptide: Any chain of amino acids, regardless of length or post-translational modification (e.g., glycosylation or phosphorylation). In one embodiment, the polypeptide is gp120 polypeptide. In one embodiment, the polypeptide is a disclosed antibody or a fragment thereof. A “residue” refers to an amino acid or amino acid mimetic incorporated in a polypeptide by an amide bond or amide bond mimetic. A polypeptide has an amino terminal (N-terminal) end and a carboxy terminal end.

Promoter: A promoter is an array of nucleic acid control sequences that directs transcription of a nucleic acid. A promoter includes necessary nucleic acid sequences near the start site of transcription, for example, in the case of a polymerase II type promoter, a TATA element. A promoter also optionally includes distal enhancer or repressor elements which can be located as much as several thousand base pairs from the start site of transcription. Both constitutive and inducible promoters are included (see for example, Bitter et al., Methods in Enzymology 153:516-544, 1987).

Specific, non-limiting examples of promoters include promoters derived from the genome of mammalian cells (such as the metallothionein promoter) or from mammalian viruses (such as the retrovirus long terminal repeat; the adenovirus late promoter; the vaccinia virus 7.5K promoter) may be used. Promoters produced by recombinant DNA or synthetic techniques may also be used. A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

Purified: The term purified does not require absolute purity; rather, it is intended as a relative term. Thus, for example, a purified peptide preparation is one in which the peptide or protein (such as an antibody) is more enriched than the peptide or protein is in its natural environment within a cell. In one embodiment, a preparation is purified such that the protein or peptide represents at least 50% of the total peptide or protein content of the preparation, such as at least 80%, at least 90%, at least 95% or greater of the total peptide or protein content.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques.

Sequence identity: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Homologs and variants of a V_{L} or a V_{H} of an antibody that specifically binds a polypeptide are typically characterized by possession of at least about 75%, for example at least about 80%, 85%, 90%, 91%, 92%, 93%, 94%, 95%, 96%, 97%, 98% or 99% sequence identity counted over the full length alignment with the amino acid sequence of interest. Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Specifically bind: When referring to an antibody, refers to a binding reaction which determines the presence of a target protein, peptide, or polysaccharide in the presence of a heterogeneous population of proteins and other biologics. Thus, under designated conditions, an antibody binds preferentially to a particular target protein, peptide or polysaccharide (such as an antigen present on the surface of a pathogen, for example gp120) and does not bind in a significant amount to other proteins or polysaccharides present in the sample or subject. Specific binding can be determined by methods known in the art. With reference to an antibody antigen complex, specific binding of the antigen and antibody has a K_{d} of less than about 10⁻⁶ Molar, such as less than about 10⁻⁶ Molar, 10⁻⁷ Molar, 10⁻⁸ Molar, 10⁻⁹, or even less than about 10⁻¹⁰ Molar.

T Cell: A white blood cell critical to the immune response. T cells include, but are not limited to, CD4⁺ T cells and CD8⁺ T cells. A CD4⁺ T lymphocyte is an immune cell that carries a marker on its surface known as “cluster of differentiation 4” (CD4). These cells, also known as helper T cells, help orchestrate the immune response, including antibody responses as well as killer T cell responses. CD8⁺ T cells carry the “cluster of differentiation 8” (CD8) marker. In one embodiment, a CD8 T cells is a cytotoxic T lymphocytes. In another embodiment, a CD8 cell is a suppressor T cell.

Therapeutic agent: Used in a generic sense, it includes treating agents, prophylactic agents, and replacement agents. A therapeutic agent is used to ameliorate a specific set of conditions in a subject with a disease or a disorder.

Therapeutically effective amount: A quantity of a specific substance, such as a disclosed antibody, sufficient to achieve a desired effect in a subject being treated. For instance, this can be the amount necessary to inhibit HIV replication or treat HIV infection. In several embodiments, a therapeutically effective amount is the amount necessary to reduce a sign or symptom of HIV infection, and/or to decrease viral titer in a subject. When administered to a subject, a dosage will generally be used that will achieve target tissue concentrations that has been shown to achieve a desired in vitro effect.

Toxin: An effector molecule that induces cytotoxicity when it contacts a cell. Specific, non-limiting examples of toxins include, but are not limited to, abrin, ricin, auristatins (such as monomethyl auristatin E (MMAE; see for example, Francisco et al., Blood, 102: 1458-1465, 2003)) and monomethyl auristatin F (MMAF; see, for example, Doronina et al., BioConjugate Chem., 17: 114-124, 2006), maytansinoids (such as DM1; see, for example, Phillips et al., Cancer Res., 68:9280-9290, 2008), Pseudomonas exotoxin (PE, such as PE35, PE37, PE38, and PE40), diphtheria toxin (DT), botulinum toxin, saporin, restrictocin or gelonin, or modified toxins thereof, or other toxic agents that directly or indirectly inhibit cell growth or kill cells. For example, PE and DT are highly toxic compounds that typically bring about death through liver toxicity. PE and DT, however, can be modified into a form for use as an immunotoxin by removing the native targeting component of the toxin (such as the domain Ia of PE and the B chain of DT) and replacing it with a different targeting moiety, such as an antibody.

Under conditions sufficient for: A phrase that is used to describe any environment that permits a desired activity. In one example the desired activity is formation of an immune complex. In particular examples the desired activity is treatment of HIV infection.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements known in the art.

Virus: Microscopic infectious organism that reproduces inside living cells. A virus consists essentially of a core of a single nucleic acid surrounded by a protein coat, and has the ability to replicate only inside a living cell. “Viral replication” is the production of additional virus by the occurrence of at least one viral life cycle. A virus may subvert the host cells' normal functions, causing the cell to behave in a manner determined by the virus. For example, a viral infection may result in a cell producing a cytokine, or responding to a cytokine, when the uninfected cell does not normally do so.

“Retroviruses” are RNA viruses wherein the viral genome is RNA. When a host cell is infected with a retrovirus, the genomic RNA is reverse transcribed into a DNA intermediate which is integrated very efficiently into the chromosomal DNA of infected cells. The integrated DNA intermediate is referred to as a provirus. The term “lentivirus” is used in its conventional sense to describe a genus of viruses containing reverse transcriptase. The lentiviruses include the “immunodeficiency viruses” which include human immunodeficiency virus (HIV) type 1 and type 2 (HIV-I and HIV-II), simian immunodeficiency virus (SIV), and feline immunodeficiency virus (FIV).

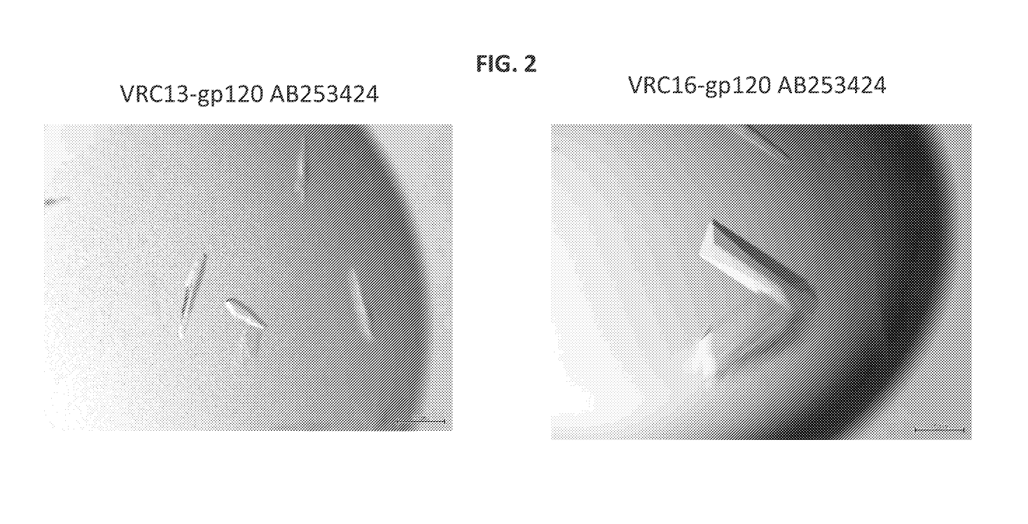

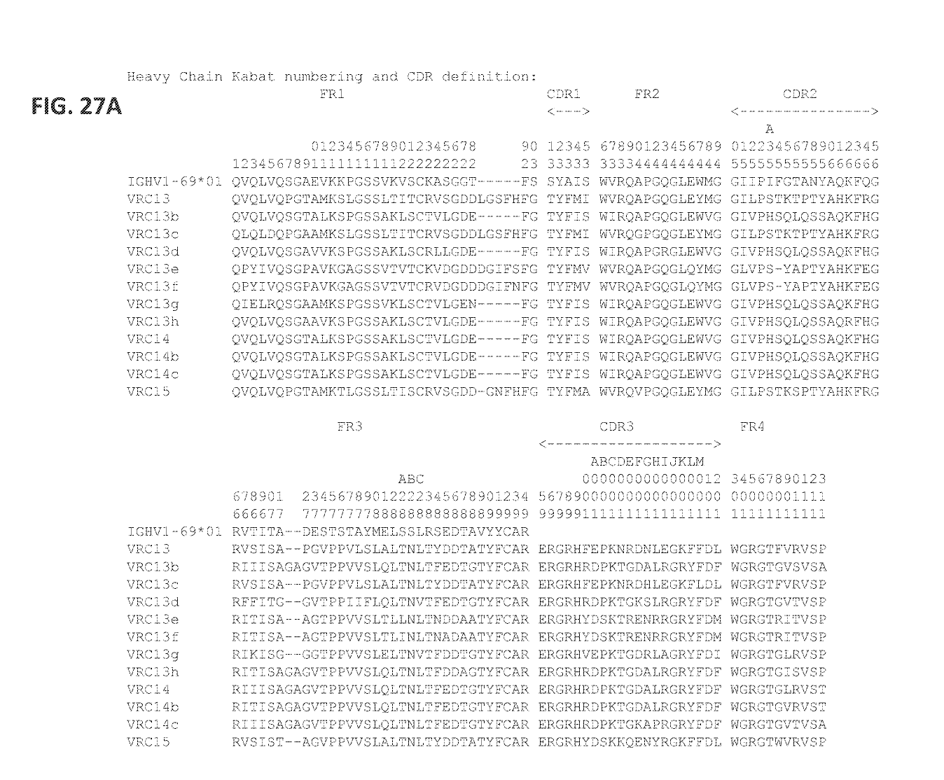

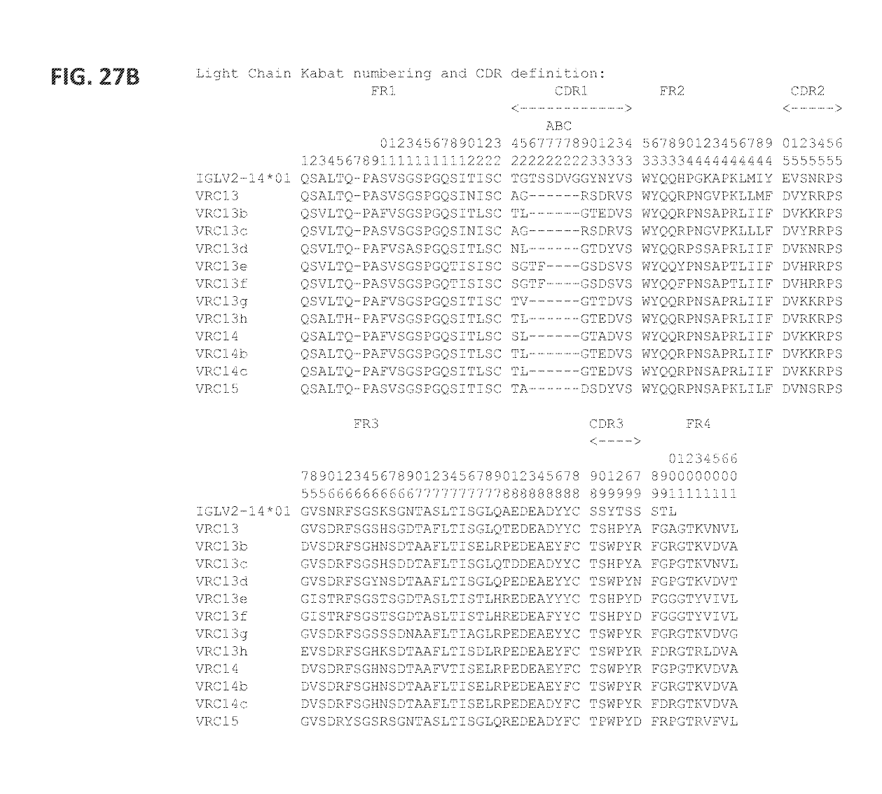

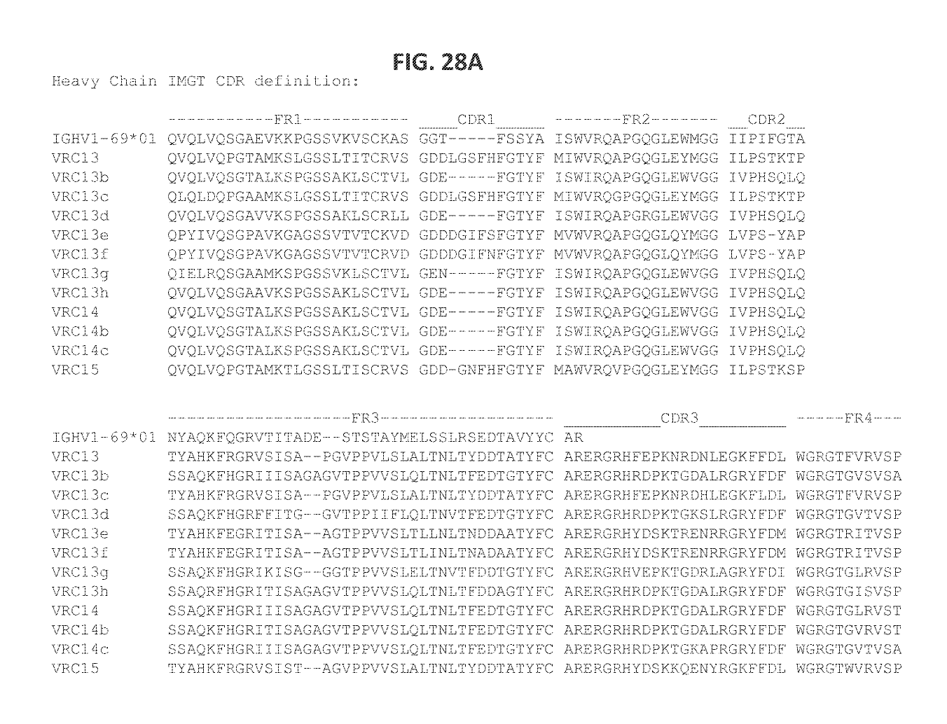

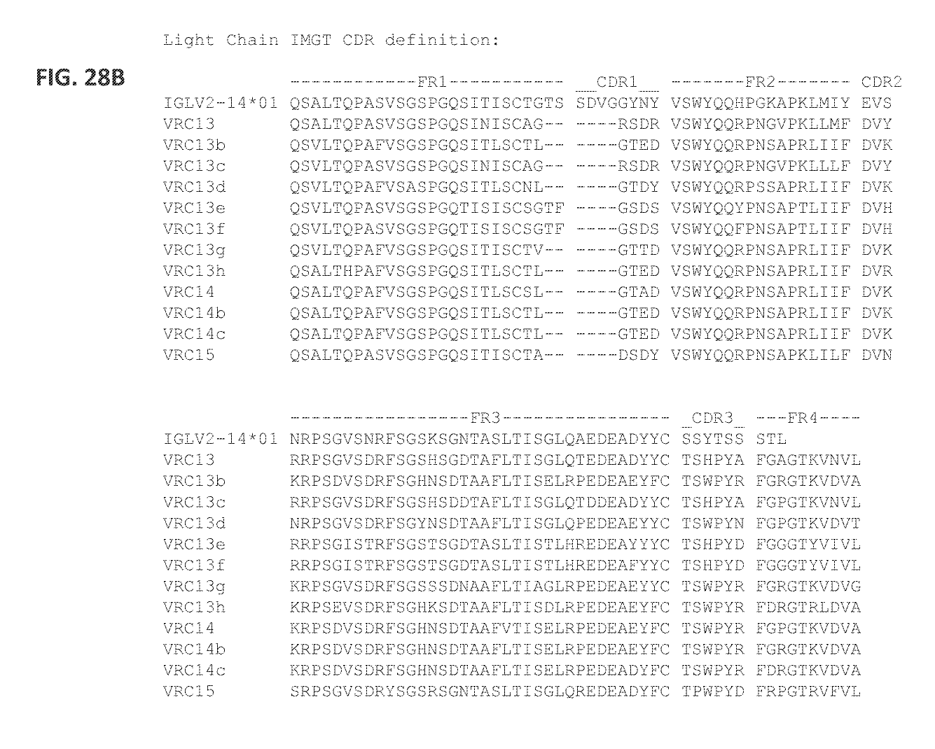

VRC01-like antibody: Exemplary VRC-01 antibodies, and methods for identifying and producing these antibodies, are disclosed, for example, in International (PCT) App. Nos. PCT/US2010/050295 and PCT/US2012/030465, and Kwong et al., Immunity, 37:412-425, 2012, each of which is incorporated herein by reference in its entirety. Generally, these antibodies bind to the CD4 binding surface of gp120 in substantially the same orientation as VRC01, and are broadly neutralizing. The coordinates of the three-dimensional structure of VRC01 bound to gp120 are available as Protein Data Bank (PDB) accession number 3NGB, incorporated by reference herein as present in PDB on Sep. 5, 2012. VRC01-like antibodies partially mimic the binding of the CD4 receptor to gp120, with an about 6 Å shift and an about 43 degree rotation from the CD4-defined position (see FIG. 2d of Zhou et al., “Structural Basis for Broad and Potent Neutralization of HIV-1 by Antibody VRC01, Science 329, 811-817 (2010), which is incorporated herein by reference in its entirety, and Protein Data Bank (PDB) accession number 3NGB). VRC13 Antibodies: A set of related antibodies including the VRC13, VRC13b, VRC13c, VRC13d, VRC13e, VRC13f, VRC13g, VRC13h, VRC14, VRC14b, VRC14c, and VRC15 antibodies as described herein. Exemplary amino acid sequences of VRC13 antibodies are provided in FIGS. 27-28. An exemplary consensus sequence for the heavy and light chain variable regions of VRC 13 antibodies is provided as SEQ ID NOs: 1 and 2, respectively. In some embodiments, these antibodies include a heavy chain variable region that is a clonal variant from donor 44 with a heavy chain variable region set forth as SEQ ID NO: 5, wherein the a heavy chain is derived from a VH1-69 gene and a VJ-2 J gene, and light chain variable region that is a clonal variant from donor 44 with a light chain variable region set forth as SEQ ID NO: 17, wherein the light chain is derived a LV2-14 V gene and a LJ-1 J gene.

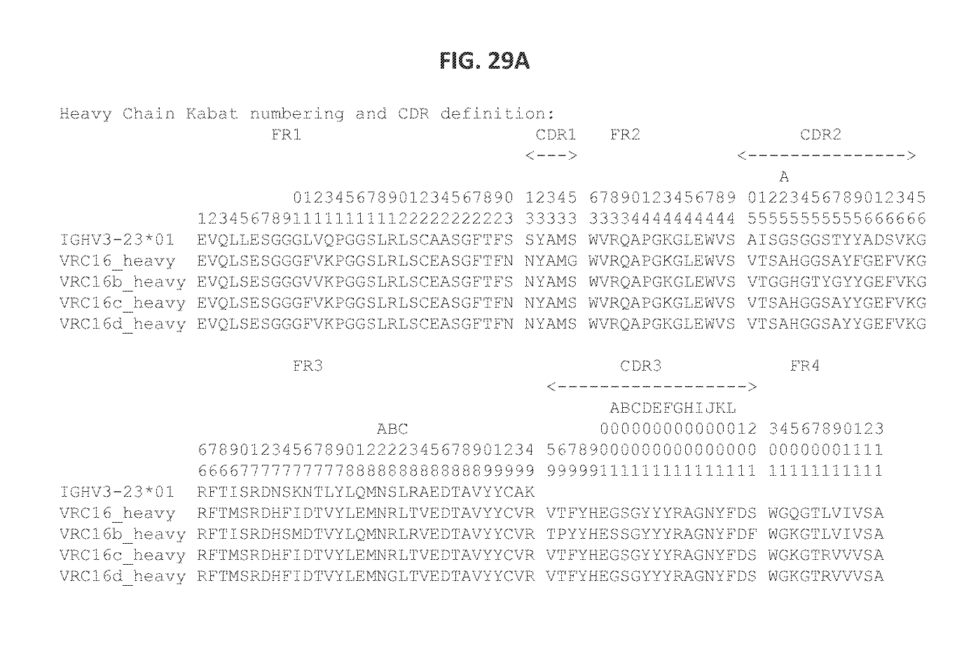

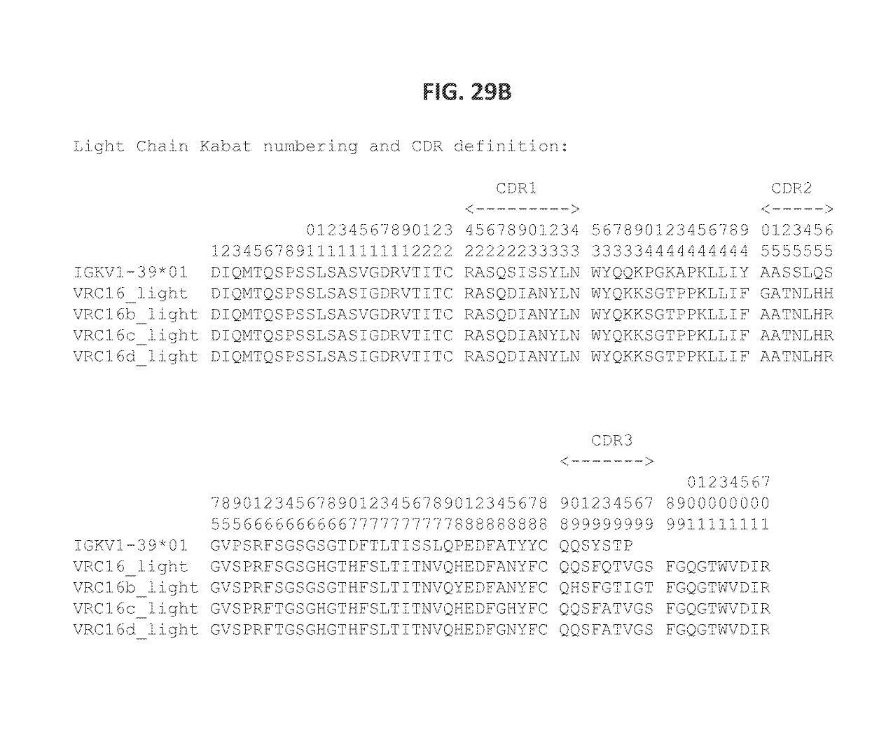

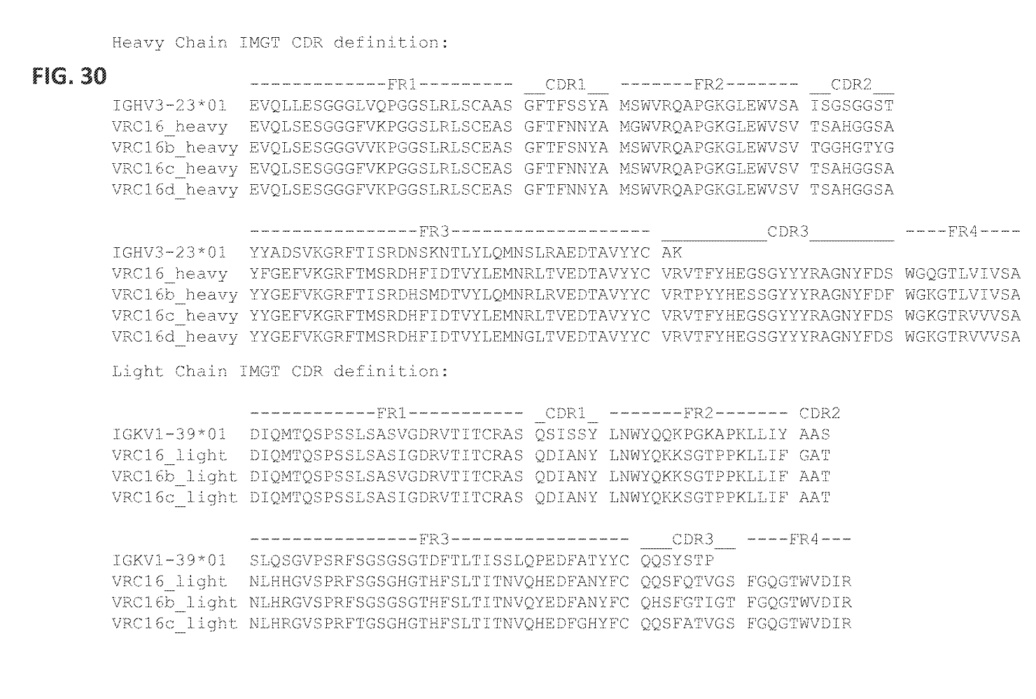

VRC16 Antibodies: A set of related antibodies including the VRC16, VRC16b, VRC16c and VRC16d antibodies as described herein. Exemplary amino acid sequences of VRC16 antibodies are provided in FIGS. 29-30. An exemplary consensus sequence for the heavy and light chain variable regions of VRC13 antibodies is provided as SEQ ID NOs: 3 and 4, respectively. In some embodiments, these antibodies include a heavy chain variable region that is a clonal variant of a heavy chain variable region set forth as SEQ ID NO: 29, wherein the heavy chain is from donor 200-3844, with a VH3-23 V gene and a VJ-1 J gene; and include light chain variable region that is a clonal variant of the light chain set forth as SEQ ID NO: 33, the light chain variable region is a clonal variant from donor 200-384, is derived from a KV1-39 V gene and a KJ-1 J gene.

### II. DESCRIPTION OF SEVERAL EMBODIMENTS

### A. Neutralizing Monoclonal Antibodies

Isolated monoclonal antibodies that specifically bind gp120 are disclosed herein. The antibodies can be fully human. Also disclosed herein are compositions including these monoclonal antibodies and a pharmaceutically acceptable carrier. Nucleic acids encoding these antibodies, expression vectors comprising these nucleic acids, and isolated host cells that express the nucleic acids are also provided.

Compositions comprising the monoclonal antibodies specific for gp120 can be used for research, diagnostic and therapeutic purposes. For example, the monoclonal antibodies disclosed herein can be used to diagnose or treat a subject having an HIV-1 infection and/or AIDS. For example, the antibodies can be used to determine HIV-1 titer in a subject. The antibodies disclosed herein also can be used to study the biology of the human immunodeficiency virus.

In several embodiments, the monoclonal antibodies include a heavy chain comprising a heavy chain complementarity determining region (HCDR)1, a HCDR2 and an HCDR3, and a light chain comprising a light chain complementarity determining region (LCDR) 1, LCDR2 and LCDR3. The disclosed antibodies specifically bind to an epitope on the surface of gp120 and are neutralizing.

In some embodiments, the HCDR3 is 22 or 23 amino acids in length and contributes at least 60% of gp120 binding surface area. Thus, in specific non-limiting examples, the HCDR3 is primarily responsible for binding to gp120. The heavy chain variable region of these antibodies has an orientation relative to gp120 when the antibody is specifically bound to gp120, and wherein the orientation is rotated by 45 degrees and translated ˜10 Å from that of a VRC01 heavy chain variable region when the VRC01 antibody is specifically bound to gp120 as shown in Protein Data Bank (PDB) accession number 3NGB.

Two classes of monoclonal antibodies, VRC13 and VRC16, are disclosed herein that bind on to an epitope on the surface of gp120 and are neutralizing. Generally, the VRC13 and VRC16 antibodies each include a variable heavy (V_{H}) and a variable light (V_{L}) chain and specifically bind gp120.

The discussion of monoclonal antibodies below refers to isolated monoclonal antibodies that include heavy and light chain variable domains including at least one complementarity determining region (CDR), such as a CDR1, CDR2 and CDR3. The person of ordinary skill in the art will understand that various CDR numbering schemes (such as the Kabat, Chothia or IMGT numbering schemes) can be used to determine CDR positions. The amino acid sequence and the CDR positions of the heavy and light chain of the VRC13, VRC13b, VRC13c, VRC13d, VRC13e, VRC13f, VRC13g, VRC13h, VRC14, VRC14b, VRC14c, and VRC15 monoclonal antibodies according to the Kabat and IMGT numbering schemes are shown in FIG. 27 (Kabat) and FIG. 28 (IMGT). The Kabat and IMGT CDR positions relative to the corresponding SEQ ID NOs for these antibodies is also listed in Table 1 (Kabat) and Table 2 (IMGT), below. The heavy and light chain CDR positions of the VRC16, VRC16b, VRC16c, and VRC16d monoclonal antibodies according to the Kabat and IMGT numbering scheme are shown in FIG. 29 (Kabat) and FIG. 30 (IMGT). The Kabat and IMGT CDR positions relative to the corresponding SEQ ID NOs for these antibodies is also listed in Table 3 (Kabat) and Table 4 (IMGT), below. The person of skill in the art will readily understand use of various CDR numbering schemes when referencing particular amino acids of the antibodies disclosed herein.

### 1. Exemplary VRC13 Antibodies

VRC13 antibodies include at least the related antibodies including the VRC13, VRC13b, VRC13c, VRC13d, VRC13e, VRC13f, VRC13g, VRC13h, VRC14, VRC14b, VRC14c, and VRC15 antibodies disclosed herein. The amino acid sequences and Kabat and IMGT CDR positions of these VRC13 antibodies are provided in FIGS. 27-28. Further the amino acid sequences of the VRC13, VRC13b, VRC13c, VRC13d, VRC13e, VRC13f, VRC13g, VRC13h, VRC14, VRC14b, VRC14c, and VRC15 heavy chain variable regions are disclosed as SEQ ID NOs: 5-16, respectively, and the amino acid sequences of the VRC13, VRC13b, VRC13c, VRC13d, VRC13e, VRC13f, VRC13g, VRC13h, VRC14, VRC14b, VRC14c, and VRC15 light chain variable regions are disclosed as SEQ ID NOs: 17-28, respectively. The amino acid residues of these SEQ ID NOs that correspond to the Kabat and IMGT CDR positions of thee VRC13 antibodies are provided in Table 1 (Kabat) and Table 2, below. A consensus CDR sequence for the heavy and light chains of the VRC13 antibodies is provided as SEQ ID NOs: 1 and 2, respectively.

In several embodiments, the monoclonal antibodies include a VRC13 heavy chain comprising a heavy chain complementarity determining region (HCDR)1, a HCDR2 and an HCDR3, and optionally a VRC13 light chain comprising a light chain complementarity determining region (LCDR) 1, LCDR2 and LCDR3. The disclosed antibodies specifically bind to an epitope on the surface of gp120 and are neutralizing. In some embodiments, the antibody is a VRC13-like antibody, such as VRC13, VRC13b, VRC13c, VRC13d, VRC13e, VRC13f, VRC13g, VRC13h, VRC14, VRC14b, VRC14c, or VRC15, that specifically binds to an epitope on the surface of gp120 and is neutralizing. In several embodiments, the VRC13-like antibody neutralizes HIV-1 strains that are resistant to neutralization by VRC01. In some embodiments, a VRC13 antibody includes a HCDR3 that binds the b19 loop of gp120 when the antibody is specifically bound to gp120.

TABLE 1
Locations of Kabat CDRs in VRC13, VRC13b, VRC13c,
VRC13d, VRC13e, VRC13f, VRC13g, VRC13h, VRC14,
VRC14b, VRC14c, or VRC15 antibodies.

| | Amino acid residues | | | |
|---|---|---|---|---|
| Antibody | HCDR1 | HCDR2 | HCDR3 | SEQ ID NO |
| VRC13 | 36-40 | 55-71 | 104-124 | 5 |
| VRC13b | 31-35 | 50-66 | 101-121 | 6 |
| VRC13c | 36-40 | 55-71 | 104-124 | 7 |
| VRC13d | 31-35 | 50-66 | 99-119 | 8 |
| VRC13e | 36-40 | 55-71 | 103-123 | 9 |
| VRC13f | 36-40 | 55-71 | 103-123 | 10 |
| VRC13g | 31-35 | 50-66 | 99-119 | 11 |
| VRC13h | 31-35 | 50-66 | 101-121 | 12 |
| VRC14 | 31-35 | 50-66 | 101-121 | 13 |
| VRC14b | 31-35 | 50-66 | 101-121 | 14 |
| VRC14c | 31-35 | 50-66 | 101-121 | 15 |
| VRC15 | 35-39 | 54-70 | 103-123 | 16 |
| Kabat | 31-35B | 50-65 | 95-102 | |
| positions | | | | |

| | Amino acid residues | | | |
|---|---|---|---|---|
| Antibody | LCDR1 | LCDR2 | LCDR3 | SEQ ID NO |
| VRC13 | 23-30 | 46-52 | 85-90 | 17 |
| VRC13b | 23-30 | 46-52 | 85-90 | 18 |
| VRC13c | 23-30 | 46-52 | 85-90 | 19 |
| VRC13d | 23-30 | 46-52 | 85-90 | 20 |
| VRC13e | 23-32 | 48-54 | 87-92 | 21 |
| VRC13f | 23-32 | 48-54 | 87-92 | 22 |
| VRC13g | 23-30 | 46-52 | 85-90 | 23 |
| VRC13h | 23-30 | 46-52 | 85-90 | 24 |
| VRC14 | 23-30 | 46-52 | 85-90 | 25 |
| VRC14b | 23-30 | 46-52 | 85-90 | 26 |
| VRC14c | 23-30 | 46-52 | 85-90 | 27 |
| VRC15 | 23-30 | 46-52 | 85-90 | 28 |
| Kabat | 24-34 | 50-56 | 89-97 | |
| positions | | | | |

TABLE 2
Locations of IMGT CDRs in VRC13, VRC13b, VRC13c,
VRC13d, VRC13e, VRC13f, VRC13g, VRC13h, VRC14,
VRC14b, VRC14c, or VRC15 antibodies.

| | Amino acid residues | | | |
|---|---|---|---|---|
| Antibody | HCDR1 | HCDR2 | HCDR3 | SEQ ID NO |
| VRC13 | 26-38 | 56-63 | 102-124 | 5 |
| VRC13b | 26-33 | 51-58 | 99-121 | 6 |
| VRC13c | 26-38 | 56-63 | 102-124 | 7 |
| VRC13d | 26-33 | 51-58 | 97-119 | 8 |
| VRC13e | 26-38 | 56-62 | 101-123 | 9 |
| VRC13f | 26-38 | 56-62 | 101-123 | 10 |
| VRC13g | 26-33 | 51-58 | 97-119 | 11 |
| VRC13h | 26-33 | 51-58 | 99-121 | 12 |
| VRC14 | 26-33 | 51-58 | 99-121 | 13 |
| VRC14b | 26-33 | 51-58 | 99-121 | 14 |
| VRC14c | 26-33 | 51-58 | 99-121 | 15 |
| VRC15 | 26-37 | 55-62 | 101-123 | 16 |

| | Amino acid residues | | | |
|---|---|---|---|---|
| | LCDR1 | LCDR2 | LCDR3 | SEQ ID NO |
| VRC13 | 25-28 | 46-48 | 85-90 | 17 |
| VRC13b | 25-28 | 46-48 | 85-90 | 18 |
| VRC13c | 25-28 | 46-48 | 85-90 | 19 |
| VRC13d | 25-28 | 46-48 | 85-90 | 20 |
| VRC13e | 27-30 | 48-50 | 87-92 | 21 |
| VRC13f | 27-30 | 48-50 | 87-92 | 22 |
| VRC13g | 25-28 | 46-48 | 85-90 | 23 |
| VRC13h | 25-28 | 46-48 | 85-90 | 24 |
| VRC14 | 25-28 | 46-48 | 85-90 | 25 |
| VRC14b | 25-28 | 46-48 | 85-90 | 26 |
| VRC14c | 25-28 | 46-48 | 85-90 | 27 |
| VRC15 | 25-28 | 46-48 | 85-90 | 28 |

In some embodiments, the antibody includes a heavy chain variable region a, that includes a heavy chain complementarity determining region (HCDR) 1, a HCD2, and/or a HCDR3, corresponding to Kabat positions 31-35B, 50-65 and 95-102, respectively, of the amino acid sequence set forth as SEQ ID NO: 1, which is a VRC13 heavy chain consensus CDR sequence:

(SEQ ID NO: 1)
QVQLVQPGTAMKSLGSSLTITCRVSGDDLGSFHFGTYFX₁X₂WVRQAPGQ
GLEYMGGX₃X₄PX₅X₆X₇X₈X₉X₁₀X₁₁AX₁₂KFX₁₃GRVSISAPGVPPVLSLAL
TNLTYDDTATYFCARERGRHX₁₄X₁₅X₁₆KX₁₇X₁₈X₁₉X₂₀X₂₁X₂₂GX₂₃X₂₄X₂₅
DX₂₆WGRGTFVRVSP

wherein X₁ is M or I; X₂ is I, S, V, or A; X₃ is I or L; X₄ is L or V; X₅ is S or H; X₆ is T, S or no amino acid; X₇ is K, Q or Y; X₈ is T, L, A or S; X₉ is P or Q; X₁₀ is T or S; X₁₁ is Y or S; X₁₂ is H or Q; X₁₃ is R or H; X14 is F, R, Y or V; X₁₅ is E or D; X₁₆ is P or S; X₁₇ is N, T or K; X₁₈ is R, G, or Q; X₁₉ is D, K or E; X₂₀ is N, A, H, S R; X₂₁ is L, R or P; X₂₂ is E, R or A; X₂₃ is K or R; X₂₄ is F or Y; X₂₅ is F or L; and X₂₆ is L, M, F or I. In other embodiments, the antibody includes a heavy chain variable region and optionally a light chain variable region, wherein the heavy chain variable region includes a heavy chain complementarity determining region (HCDR) 1, a HCD2, and a HCDR3, corresponding to Kabat positions 31-35B, 50-65 and 95-102, respectively, of the amino acid sequence set forth as SEQ ID NO: 1.

In several embodiments, the antibody includes a HCDR1, a HCD2, and/or a HCDR3, corresponding to Kabat positions 31-35B, 50-65 and 95-102, respectively, of one of the amino acid sequences set forth as SEQ ID NOs: 5-16. In additional embodiments, the VRC13 antibody includes a HCDR1, a HCD2, and a HCDR3, corresponding to positions 31-35B, 50-65 and 95-102, respectively, of one of the amino acid sequences set forth as SEQ ID NOs: 5-16.

In some embodiments, the heavy chain variable region comprises the amino acid sequence set forth as SEQ ID NO: 1. In specific non-limiting examples, the heavy chain variable region comprises the amino acid sequence set forth as one of SEQ ID NOs: 5-16.

In additional embodiments, the VRC13 antibody includes a light chain variable region comprises a light chain complementarity determining region (LCDR) 1, a LCDR2 and/or an LCDR3 corresponding to Kabat positions 24-34, 50-56, and 89-97, respectively, of the VRC light chain consensus amino acid sequence set forth as SEQ ID NO: 2, which is a VRC13 light chain consensus CDR sequence:

(SEQ ID NO: 2)
QSALTQPASVSGSPGQSINISCX₁X₂X₃X₄DX₅X₆SWYQQRPNGVPKLLM
FDVX₇X₈RPSGVSDRFSGSHSGDTAFLTISGLQTEDEADYYCTX₉X₁₀PY
X₁₁FGAGTKVNVL

wherein X₁ is A, T, N, or S; X₂ is G, L, GTF, V, L or A; X₃ is R, G, or D; X₄ is S or T; X₅ is D, E, T, or A; X₆ is R, D, Y, or S; X₇ is Y, K, H, R or N; X₈ is R, K, N, or S; X₉ is; S or P; X₁₀ is H or W; and X₁ᵢis A, R, N, or D. In further embodiments, the VRC13 antibody includes a light chain variable region comprising a light chain complementarity determining region (LCDR) 1, a LCDR2 and an LCDR3 corresponding to Kabat positions 24-34, 50-56, and 89-97, respectively, of the VRC light chain consensus amino acid sequence set forth as SEQ ID NO: 2.

In additional embodiments, the isolated monoclonal antibody includes a LCDR1, a LCDR2, and/or an LCDR3 corresponding to Kabat positions 24-34, 50-56, and 89-97, respectively, of one of the amino acid sequences set forth as SEQ ID NO: 17-28. In yet other embodiments, the isolated monoclonal antibody includes a LCDR1, a LCDR2, and an LCDR3 corresponding to positions Kabat 24-34, 50-56, and 89-97, respectively, of one of the amino acid sequences set forth as SEQ ID NO: 17-28.

In yet other embodiments, the isolated monoclonal antibody includes a light chain variable region comprising the amino acid sequence set forth as SEQ ID NO: 2. In several specific non-limiting examples, the light chain variable region comprises the amino acid sequence set forth as one of SEQ ID NOs: 17-28.

The VRC 13 heavy and the light chains can be used in any combination. In some embodiments, the heavy chain of the human monoclonal antibody includes an amino acid sequence having at least 80% (such as at least 85%, 90%, 91%, 92%, 93%, 94%, 95%, 96%, 97%, 98%, or at least 99%) sequence identity with the amino acid sequence set forth as any one of SEQ ID NOs: 1 or 5-16. In further embodiments, the light chain of the human monoclonal antibody includes the amino acid sequence having at least 80% (such as at least 85%, 90%, 91%, 92%, 93%, 94%, 95%, 96%, 97%, 98%, or at least 99%) sequence identity with the amino acid sequence set forth as any one of SEQ ID NOs: 2 or 17-28.

In some embodiments, the antibody includes a heavy chain variable region including a HCDR3 including the amino acid sequence of the HCDR3 of one of the VRC13, VRC13b, VRC13c, VRC13d, VRC13e, VRC13f, VRC13g, VRC13h, VRC14, VRC14b, VRC14c, or VRC15 antibodies as shown in FIG. 27 or FIG. 28. In other embodiments, the antibody includes a heavy chain variable region including the amino acid sequence of the HCDR1, HCDR2 and/or HCDR3 of one of the VRC13, VRC13b, VRC13c, VRC13d, VRC13e, VRC13f, VRC13g, VRC13h, VRC14, VRC14b, VRC14c, or VRC15 antibodies as shown in FIG. 27 or FIG. 28. In additional embodiments, the antibody includes a heavy chain variable region including the amino acid sequence of the HCDR1, HCDR2, and HCDR3 of one of the VRC13, VRC13b, VRC13c, VRC13d, VRC13e, VRC13f, VRC13g, VRC13h, VRC14, VRC14b, VRC14c, or VRC15 antibodies as shown in FIG. 27 or FIG. 28.

In some embodiments, the antibody includes a light chain variable region including one or more of the light chain CDRs of the VRC13, VRC13b, VRC13c, VRC13d, VRC13e, VRC13f, VRC13g, VRC13h, VRC14, VRC14b, VRC14c, or VRC15 antibodies as shown in FIG. 27 or FIG. 28. In other embodiments, the antibody includes a light chain variable region including the amino acid sequence of the LCDR1, LCDR2, and/or LCDR3 of one of the VRC13, VRC13b, VRC13c, VRC13d, VRC13e, VRC13f, VRC13g, VRC13h, VRC14, VRC14b, VRC14c, or VRC15 antibodies as shown in FIG. 27 or FIG. 28. In additional embodiments, the antibody includes a light chain variable region including the amino acid sequence of the LCDR1, LCDR2, and LCDR3 of one of the VRC13, VRC13b, VRC13c, VRC13d, VRC13e, VRC13f, VRC13g, VRC13h, VRC14, VRC14b, VRC14c, or VRC15 antibodies as shown in FIG. 27 or FIG. 28.

In some embodiments, the antibody includes a heavy chain variable region including the amino acid sequence of the HCDR1, HCDR2 and/or HCDR3 of one of the VRC13, VRC13b, VRC13c, VRC13d, VRC13e, VRC13f, VRC13g, VRC13h, VRC14, VRC14b, VRC14c, or VRC15 antibodies as shown in FIG. 27 or FIG. 28, and a light chain variable region including the amino acid sequence of the LCDR1, LCDR2 and/or LCDR3 of one of the VRC13, VRC13b, VRC13c, VRC13d, VRC13e, VRC13f, VRC13g, VRC13h, VRC14, VRC14b, VRC14c, or VRC15 antibodies, respectively, as shown in FIG. 27 or FIG. 28. In other embodiments, the antibody includes a heavy chain variable region including the amino acid sequence of the HCDR1, HCDR2, and HCDR3 of one of the VRC13, VRC13b, VRC13c, VRC13d, VRC13e, VRC13f, VRC13g, VRC13h, VRC14, VRC14b, VRC14c, or VRC15 antibodies as shown in FIG. 27 or FIG. 28, and a light chain variable region including the amino acid sequence of the LCDR1, LCDR2, and LCDR3 of one of the VRC13, VRC13b, VRC13c, VRC13d, VRC13e, VRC13f, VRC13g, VRC13h, VRC14, VRC14b, VRC14c, or VRC15 antibodies as shown in FIG. 27 or FIG. 28. In additional embodiments, the antibody includes a heavy chain variable region and a light chain variable region including the amino acid sequence of the HCDR1, HCDR2, and HCDR3, and the LCDR1, LCDR2, and LCD3, of one of the VRC13, VRC13b, VRC13c, VRC13d, VRC13e, VRC13f, VRC13g, VRC13h, VRC14, VRC14b, VRC14c, or VRC15 antibody as shown in FIG. 27 or FIG. 28. In additional embodiments, the antibody includes a heavy chain variable region and a light chain variable region including the amino acid sequence of the HCDR1, HCDR2, and HCDR3, and the LCDR1, LCDR2, and LCD3, of the VRC13 antibody as shown in FIG. 27 or FIG. 28. In additional embodiments, the antibody includes a heavy chain variable region and a light chain variable region including the amino acid sequence of the HCDR1, HCDR2, and HCDR3, and the LCDR1, LCDR2, and LCD3, of the VRC13b antibody as shown in FIG. 27 or FIG. 28. In additional embodiments, the antibody includes a heavy chain variable region and a light chain variable region including the amino acid sequence of the HCDR1, HCDR2, and HCDR3, and the LCDR1, LCDR2, and LCD3, of the VRC13c antibody as shown in FIG. 27 or FIG. 28. In additional embodiments, the antibody includes a heavy chain variable region and a light chain variable region including the amino acid sequence of the HCDR1, HCDR2, and HCDR3, and the LCDR1, LCDR2, and LCD3, of the VRC13d antibody as shown in FIG. 27 or FIG. 28. In additional embodiments, the antibody includes a heavy chain variable region and a light chain variable region including the amino acid sequence of the HCDR1, HCDR2, and HCDR3, and the LCDR1, LCDR2, and LCD3, of the VRC13e antibody as shown in FIG. 27 or FIG. 28. In additional embodiments, the antibody includes a heavy chain variable region and a light chain variable region including the amino acid sequence of the HCDR1, HCDR2, and HCDR3, and the LCDR1, LCDR2, and LCD3, of the VRC13f antibody as shown in FIG. 27 or FIG. 28. In additional embodiments, the antibody includes a heavy chain variable region and a light chain variable region including the amino acid sequence of the HCDR1, HCDR2, and HCDR3, and the LCDR1, LCDR2, and LCD3, of the VRC13g antibody as shown in FIG. 27 or FIG. 28. In additional embodiments, the antibody includes a heavy chain variable region and a light chain variable region including the amino acid sequence of the HCDR1, HCDR2, and HCDR3, and the LCDR1, LCDR2, and LCD3, of the VRC13h antibody as shown in FIG. 27 or FIG. 28. In additional embodiments, the antibody includes a heavy chain variable region and a light chain variable region including the amino acid sequence of the HCDR1, HCDR2, and HCDR3, and the LCDR1, LCDR2, and LCD3, of the VRC14 antibody as shown in FIG. 27 or FIG. 28. In additional embodiments, the antibody includes a heavy chain variable region and a light chain variable region including the amino acid sequence of the HCDR1, HCDR2, and HCDR3, and the LCDR1, LCDR2, and LCD3, of the VRC14b antibody as shown in FIG. 27 or FIG. 28. In additional embodiments, the antibody includes a heavy chain variable region and a light chain variable region including the amino acid sequence of the HCDR1, HCDR2, and HCDR3, and the LCDR1, LCDR2, and LCD3, of the VRC14c antibody as shown in FIG. 27 or FIG. 28. In additional embodiments, the antibody includes a heavy chain variable region and a light chain variable region including the amino acid sequence of the HCDR1, HCDR2, and HCDR3, and the LCDR1, LCDR2, and LCD3, of the VRC15 antibody as shown in FIG. 27 or FIG. 28.

In some embodiments, the antibody includes a heavy chain variable region including an amino acid sequence at least 80%, 85%, 90%, 91%, 92%, 93%, 94%, 95%, 96%, 97%, 98%, or 99% identical to the amino acid sequence of one of the VRC13, VRC13b, VRC13c, VRC13d, VRC13e, VRC13f, VRC13g, VRC13h, VRC14, VRC14b, VRC14c, or VRC15 antibodies as shown in FIG. 27 or FIG. 28, and optionally a light chain variable region including an amino acid sequence at least 80%, 85%, 90%, 91%, 92%, 93%, 94%, 95%, 96%, 97%, 98%, or 99% identical to the amino acid sequence of one of the VRC13, VRC13b, VRC13c, VRC13d, VRC13e, VRC13f, VRC13g, VRC13h, VRC14, VRC14b, VRC14c, or VRC15 antibodies as shown in FIG. 27 or FIG. 28.

For example, in some embodiments, the heavy chain of the human monoclonal antibody includes an amino acid sequence having at least 80% (such as at least 85%, 90%, 91%, 92%, 93%, 94%, 95%, 96%, 97%, 98%, or at least 99%) sequence identity with an amino acid sequence set forth as any one of SEQ ID NO: 5-16, and the light chain of the human monoclonal antibody includes the amino acid sequence having at least 80% (such as at least 85%, 90%, 91%, 92%, 93%, 94%, 95%, 96%, 97%, 98%, or at least 99%) sequence identity with the amino acid sequence set forth as any one of SEQ ID NOs: 17-28. In one embodiment, the heavy chain of the human monoclonal antibody includes an amino acid sequence having at least 80% (such as at least 85%, 90%, 91%, 92%, 93%, 94%, 95%, 96%, 97%, 98%, or at least 99%) sequence identity with an amino acid sequence set forth as SEQ ID NO: 5, and the light chain of the human monoclonal antibody includes the amino acid sequence having at least 80% (such as at least 85%, 90%, 91%, 92%, 93%, 94%, 95%, 96%, 97%, 98%, or at least 99%) sequence identity with the amino acid sequence set forth as SEQ ID NO: 17.

In some embodiments, the heavy chain of any one of the VRC13, VRC13b, VRC13c, VRC13d, VRC13e, VRC13f, VRC13g, VRC13h, VRC14, VRC14b, VRC14c, or VRC15 antibodies can be complemented with the light chain of any one of the VRC13, VRC13b, VRC13c, VRC13d, VRC13e, VRC13f, VRC13g, VRC13h, VRC14, VRC14b, VRC14c, or VRC15 antibodies and still retain specific binding to gp120, for example retain specific binding for the CD4 binding site of gp120, and maintain neutralizing activity.

In other embodiments, a nucleic acid sequence encoding a light chain variable region VRC13 clonal variant light chain variable domain is derived from the VL2-14 V gene and a JL 1 J gene from donor 44 and is about 10%, 15%, 20%, 25%, 30%, 35% or 40%, such as about 15% to 40% divergent, such as 25% divergent from the VL2-14 V gene and a JL 1 J gene.

In some embodiments, the antibody includes a heavy chain variable region that is a clonal variant from donor 44 of the heavy chain variable region set forth as SEQ ID NO: 5, wherein the heavy chain is derived from a VH1-69 gene and a VJ-2 J gene, and light chain variable region that is a clonal variant from donor 44 of the light chain variable region set forth as SEQ ID NO: 17, wherein the light chain is derived from a LV2-14 V gene and a LJ-1 J gene.

In some examples, a nucleic acid sequence encoding a heavy chain variable region of a VRC 13 clonal variant is derived from a VH1-69 gene and a VJ-2 J gene from donor 44 and is about 10%, 15%, 20%, 25%, 30%, 35% or 40%, such as about 15% to 40% divergent, such as 25% divergent from a VH1-69 gene and a VJ-2 J gene. In other embodiments, a nucleic acid sequence encoding a light chain variable region VRC13 clonal variant light chain variable domain is derived from the LV2-14 V gene and a LJ-1 J gene from donor 44 and is about 10%, 15%, 20%, 25%, 30%, 35% or 40%, such as about 15% to 40% divergent, such as 25% divergent from the LV2-14 and LJ-1 J gene. In some embodiments the heavy chain variable region is at least 50%, at least 60%, at least 70%, at least 80%, at least 90%, or at least 95% identical to SEQ ID NO: 5. In additional embodiments the heavy chain variable region is at least 50%, at least 60%, at least 70%, at least 80%, at least 90%, or at least 95% identical to SEQ ID NO: 17. In some embodiments a clonal variant of VRC13 can compete for binding to gp120 with an antibody including a heavy and light chain comprising SEQ ID NOs: 5 and 17, respectively.

### 2. Exemplary VRC16 Antibodies

VRC16 antibodies include at least the related antibodies including the VRC16, VRC16b, VRC16c and VRC16d antibodies as described herein. The amino acid sequences and Kabat and IMGT CDR positions of VRC16 antibodies are provided in FIGS. 29-30. Further the amino acid sequences of the VRC16, VRC16b, VRC16c and VRC16d heavy chain variable regions are disclosed as SEQ ID NOs: 29-32, respectively, and the amino acid sequences of the VRC16, VRC16b, VRC16c and VRC16d light chain variable regions are disclosed as SEQ ID NOs: 33-36, respectively. The amino acid residues of these SEQ ID NOs that correspond to the Kabat and IMGT CDR positions of the VRC16 antibodies are provided in Table 3 (Kabat) and Table 4, below. A consensus CDR sequence for the heavy and light chains of the VRC13 antibodies is provided as SEQ ID NOs: 3 and 4, respectively.

In several embodiments, the monoclonal antibodies include a VRC16 heavy chain comprising a heavy chain complementarity determining region (HCDR)1, a HCDR2 and an HCDR3, and optionally a VRC16 light chain comprising a light chain complementarity determining region (LCDR) 1, LCDR2 and LCDR3. The disclosed antibodies specifically bind to an epitope on the surface of gp120 and are neutralizing.

In some embodiments, the antibody is a VRC16-like antibody, such as VRC16, VRC16b, VRC16c, or VRC16d that specifically binds to an epitope on the surface of gp120 and is neutralizing. In several embodiments, the VRC13-like antibody neutralizes HIV-1 strains that are resistant to neutralization by VRC01.

TABLE 3
Locations of Kabat CDRs in VRC16, VRC16b,
VRC16c, and VRC16d antibodies.

| | Amino acid residues | | | |
|---|---|---|---|---|
| Antibody | HCDR1 | HCDR2 | HCDR3 | SEQ ID NO |
| VRC16 | 31-35 | 50-66 | 99-118 | 29 |
| VRC16b | 31-35 | 50-66 | 99-118 | 30 |
| VRC16c | 31-35 | 50-66 | 99-118 | 31 |
| VRC16d | 31-35 | 50-66 | 99-118 | 32 |
| Kabat | 31-35B | 50-65 | 95-102 | |
| positions | | | | |

| | Amino acid residues | | | |
|---|---|---|---|---|
| Antibody | LCDR1 | LCDR2 | LCDR3 | SEQ ID NO |
| VRC16 | 24-34 | 50-56 | 89-97 | 33 |
| VRC16b | 24-34 | 50-56 | 89-97 | 34 |
| VRC16c | 24-34 | 50-56 | 89-97 | 35 |
| VRC16d | 24-34 | 50-56 | 89-97 | 36 |
| Kabat | 24-34 | 50-56 | 89-97 | |
| positions | | | | |

TABLE 4
Locations of IMGT CDRs in VRC16, VRC16b,
VRC16c, and VRC16d antibodies.

| | Amino acid residues | | | |
|---|---|---|---|---|
| Antibody | HCDR1 | HCDR2 | HCDR3 | SEQ ID NO |
| VRC16 | 26-33 | 51-58 | 97-118 | 29 |
| VRC16b | 26-33 | 51-58 | 97-118 | 30 |
| VRC16c | 26-33 | 51-58 | 97-118 | 31 |
| VRC16d | 26-33 | 51-58 | 97-118 | 32 |
| | Amino acid residues | | | |
| | LCDR1 | LCDR2 | LCDR3 | SEQ ID NO |
| VRC16 | 27-32 | 50-52 | 89-97 | 33 |
| VRC16b | 27-32 | 50-52 | 89-97 | 34 |
| VRC16c | 27-32 | 50-52 | 89-97 | 35 |
| VRC16d | 27-32 | 50-52 | 89-97 | 36 |

In some embodiments, the isolated monoclonal antibody incudes a heavy chain variable region that comprises a heavy chain complementarity determining region (HCDR)1, a HCDR2, and/or a HCDR3 corresponding to Kabat positions 31-35B, 50-65 and 95-102, respectively, of the heavy chain CDR consensus amino acid sequence set forth as SEQ ID NO: 3, which is a VRC16 heavy chain consensus CDR sequence: EVQLSESGGGFVKPGGSLRLSCEASGFTFX₁NYAMGWVRQAPGKGLEWVSVTX₂X₃HGX₄X₅X₆YF GEFVKGRFTMSRDHFIDTVYLEMNRLTVEDTAVYYCVRX₇X₈X₉YHEX₁₀SGYYYRAGNYFDX₁₁W GQGTLVIVSA (SEQ ID NO: 3), wherein X₁ is N or S; X₂ is S or G; X₃ is A or G; X₄ is G or T; X₅ is S or Y; X₆ is A or G; X₇ is V or T; X₈ is T or P; X₉ is F or Y; X₁₀ is G or S; and X_{II} is F or S. In other embodiments, the isolated monoclonal antibody incudes a heavy chain variable region and optionally a light chain variable region, wherein the heavy chain variable region comprises a heavy chain complementarity determining region (HCDR) 1, a HCDR2, and a HCDR3 corresponding to Kabat positions 31-35B, 50-65 and 95-102, respectively, of the amino acid sequence set forth as SEQ ID NO: 3.

In further embodiments, the isolated monoclonal antibody includes a HCDR1, a HCDR2, and/or a HCDR3 corresponding to Kabat positions 31-35B, 50-65 and 95-102, respectively, of one of the amino acid sequence set forth as SEQ ID NOs: 29-32. In yet other embodiments, the isolated VRC16 monoclonal antibody incudes a HCDR1, a HCDR2, and a HCDR3 corresponding to Kabat positions 31-35B, 50-65 and 95-102, respectively, of one of the amino acid sequence set forth as SEQ ID NOs: 29-32.

In some embodiments, the heavy chain variable region comprises the amino acid sequence set forth as SEQ ID NO: 3. In specific non-limiting examples, the heavy chain variable region comprises the amino acid sequence set forth as one of SEQ ID NOs: 29-33.

In yet other embodiments, the isolated monoclonal antibody includes a light chain variable region comprises a light chain complementarity determining region (LCDR) 1, a LCDR2, and/or a LCDR3 corresponding to Kabat positions 24-34, 50-56, and 89-97, respectively, of the amino acid sequence set forth as SEQ ID NO: 4, which is a VRC16 light chain consensus CDR sequence: DIQMTQSPSSLSASIGDRVTITCRASQDIANYLNWYQKKSGTPPKLLIFX₁ATNLHX₂GVSPRFSGSG HGTHFSLTITNVQHEDFANYFCQX₃SFX₄TX₅GX₆FGQGTWVDIR (SEQ ID NO: 4), wherein X₁ is G or A; X₂ is H or R; X₃ is Q or H; X₄ is Q, G or A; X₅ is V or I; and X₆ is S or T. In further embodiments, the VRC16 antibody includes a light chain variable region comprising a light chain complementarity determining region (LCDR) 1, a LCDR2 and an LCDR3 corresponding to Kabat positions 24-34, 50-56, and 89-97, respectively, of the VRC light chain consensus amino acid sequence set forth as SEQ ID NO: 4.

In some embodiments, the isolated monoclonal antibody includes a LCDR1, a LCDR2 and/or an LCDR3 corresponding to Kabat positions 24-34, 50-56, and 89-97, respectively, of one of the amino acid sequences set forth as SEQ ID NO: 33-36. In further embodiments, the isolated monoclonal antibody includes a LCDR1, a LCDR2 and an LCDR3 corresponding to Kabat positions 24-34, 50-56, and 89-97, respectively, of one of the amino acid sequences set forth as SEQ ID NO: 33-36.

In yet other embodiments, the isolated monoclonal antibody includes a light chain variable region comprising the amino acid sequence set forth as SEQ ID NO: 4. In several specific non-limiting examples, the light chain variable region comprises the amino acid sequence set forth as one of SEQ ID NOs: 33-36.

In some embodiments, the heavy chain of the human monoclonal antibody includes an amino acid sequence having at least 80% (such as at least 85%, 90%, 91%, 92%, 93%, 94%, 95%, 96%, 97%, 98%, or at least 99%) sequence identity with the amino acid sequence set forth as any one of SEQ ID NOs: 3 or 29-32.

The VRC 16-like heavy and the light chains can be used in any combination. In further embodiments, the light chain of the human monoclonal antibody includes the amino acid sequence having at least 80% (such as at least 85%, 90%, 91%, 92%, 93%, 94%, 95%, 96%, 97%, 98%, or at least 99%) sequence identity with the amino acid sequence set forth as any one of SEQ ID NOs: 4 or 33-36.

In some embodiments, the antibody includes a heavy chain variable region including a HCDR3 including the amino acid sequence of the HCDR3 of one of the VRC16, VRC16b, VRC16c, or VRC16d antibodies as shown in FIG. 29 or FIG. 30. In other embodiments, the antibody includes a heavy chain variable region including the amino acid sequence of the HCDR1, HCDR2 and/or HCDR3 of one of the VRC16, VRC16b, VRC16c, or VRC16d antibodies as shown in FIG. 29 or FIG. 30. In additional embodiments, the antibody includes a heavy chain variable region including the amino acid sequence of the VRC16, VRC16b, VRC16c, or VRC16d antibodies as shown in FIG. 29 or FIG. 30.

In some embodiments, the antibody includes a light chain variable region including one or more of the light chain CDRs of the VRC16, VRC16b, VRC16c or VRC16d antibodies as shown in FIG. 29 or FIG. 30. In other embodiments, the antibody includes a light chain variable region including the amino acid sequence of the LCDR1, LCDR2, and/or LCDR3 of one of the VRC16, VRC16b, VRC16c or VRC16d antibodies as shown in FIG. 29 or FIG. 30. In additional embodiments, the antibody includes a light chain variable region including the amino acid sequence of the LCDR1, LCDR2, and LCDR3 of one of the VRC16, VRC16b, VRC16c or VRC16d antibodies as shown in FIG. 29 or FIG. 30.

In some embodiments, the antibody includes a heavy chain variable region including the amino acid sequence of the HCDR1, HCDR2 and/or HCDR3 of one of the VRC16, VRC16b, VRC16c or VRC16d antibodies as shown in FIG. 29 or FIG. 30, and a light chain variable region including the amino acid sequence of the LCDR1, LCDR2 and/or LCDR3 of one of the VRC16, VRC16b, VRC16c or VRC16d antibodies as shown in FIG. 29 or FIG. 30. In other embodiments, the antibody includes a heavy chain variable region including the amino acid sequence of the HCDR1, HCDR2, and HCDR3 of one of the VRC16, VRC16b, VRC16c or VRC16d antibodies as shown in FIG. 29 or FIG. 30, and a light chain variable region including the amino acid sequence of the LCDR1, LCDR2, and LCDR3 of one of the VRC16, VRC16b, VRC16c or VRC16d antibodies as shown in FIG. 29 or FIG. 30.

In additional embodiments, the antibody includes a heavy chain variable region and a light chain variable region including the amino acid sequence of the HCDR1, HCDR2, and HCDR3, and the LCDR1, LCDR2, and LCD3, of the VRC16 antibody as shown in FIG. 29 or FIG. 30. In additional embodiments, the antibody includes a heavy chain variable region and a light chain variable region including the amino acid sequence of the HCDR1, HCDR2, and HCDR3, and the LCDR1, LCDR2, and LCD3, of the VRC16b antibody as shown in FIG. 29 or FIG. 30. In additional embodiments, the antibody includes a heavy chain variable region and a light chain variable region including the amino acid sequence of the HCDR1, HCDR2, and HCDR3, and the LCDR1, LCDR2, and LCD3, of the VRC16c antibody as shown in FIG. 29 or FIG. 30. In additional embodiments, the antibody includes a heavy chain variable region and a light chain variable region including the amino acid sequence of the HCDR1, HCDR2, and HCDR3, and the LCDR1, LCDR2, and LCD3, of the VRC16d antibody as shown in FIG. 29 or FIG. 30.

In some embodiments, the antibody includes a heavy chain variable region including an amino acid sequence at least 80%, 85%, 90%, 91%, 92%, 93%, 94%, 95%, 96%, 97%, 98%, or 99% identical to the amino acid sequence of one of the VRC16, VRC16b, VRC16c, or VRC16d antibodies as shown in FIG. 29 or FIG. 30. The antibody can also include a light chain variable region including an amino acid sequence at least 80%, 85%, 90%, 91%, 92%, 93%, 94%, 95%, 96%, 97%, 98%, or 99% identical to the amino acid sequence of one of the VRC16, VRC16b, VRC16c, or VRC16d antibodies as shown in FIG. 29 or FIG. 30.

For example, in some embodiments, the heavy chain of the human monoclonal antibody includes an amino acid sequence having at least 80% (such as at least 85%, 90%, 91%, 92%, 93%, 94%, 95%, 96%, 97%, 98%, or at least 99%) sequence identity with an amino acid sequence set forth as any one of SEQ ID No: 29-32, and the light chain of the human monoclonal antibody includes the amino acid sequence having at least 80% (such as at least 85%, 90%, 91%, 92%, 93%, 94%, 95%, 96%, 97%, 98%, or at least 99%) sequence identity with the amino acid sequence set forth as any one of SEQ ID NOs: 33-36. In one embodiment, the heavy chain of the human monoclonal antibody includes an amino acid sequence having at least 80% (such as at least 85%, 90%, 91%, 92%, 93%, 94%, 95%, 96%, 97%, 98%, or at least 99%) sequence identity with an amino acid sequence set forth as SEQ ID NO: 29, and the light chain of the human monoclonal antibody includes the amino acid sequence having at least 80% (such as at least 85%, 90%, 91%, 92%, 93%, 94%, 95%, 96%, 97%, 98%, or at least 99%) sequence identity with the amino acid sequence set forth as SEQ ID NO: 33.

In some embodiments, the heavy chain of any one of the VRC16, VRC16b, VRC16c, or VRC16d antibodies can be complemented with the light chain of any one of the VRC16, VRC16b, VRC16c, or VRC16d antibodies and still retain specific binding to gp120, for example retain specific binding for the CD4 binding site of gp120, and maintain neutralizing activity.

In some embodiments, the VRC16 antibody includes a heavy chain variable region that is a clonal variant from donor 200-384 of the heavy chain variable region set forth as SEQ ID NO: 29, wherein the heavy chain is derived from a VH3-23 V gene and a VJ-6 J gene; and a light chain variable region that is a clonal variant from donor 200-384 of the light chain variable domain set forth as SEQ ID NO: 33, wherein the heavy chain is derived from a KV1-39 V gene and a KJ-1 J gene.

In some examples, a nucleic acid sequence encoding a heavy chain variable region of a VRC 16 clonal variant is derived from a VH3-23 V gene and a VJ-6 J gene from donor 200-384 and is about 10%, 15%, 20%, 25%, 30%, 35% or 40%, such as about 15% to 40% divergent, such as 25% divergent from a VH3-23 V gene and a VJ-6 J gene. In other embodiments, a nucleic acid sequence encoding a light chain variable region VRC16 clonal variant light chain variable domain is derived from the KV1-39 V gene and a KJ-1 J gene from donor 200-384 and is about 10%, 15%, 20%, 25%, 30%, 35% or 40%, such as about 15% to 40% divergent, such as 25% divergent from the KV1-39 V gene and a KJ-1 J gene. In some embodiments the heavy chain variable region is at least 50%, at least 60%, at least 70%, at least 80%, at least 90%, or at least 95% identical to SEQ ID NO: 29. In additional embodiments the heavy chain variable region is at least 50%, at least 60%, at least 70%, at least 80%, at least 90%, or at least 95% identical to SEQ ID NO: 33. In some embodiments a clonal variant of VRC16 can compete for binding to gp120 with an antibody including a heavy and light chain comprising SEQ ID NOs: 29 and 33, respectively.

### 3. Additional Description of the Disclosed Antibodies

Without being bound by theory, it is believed that the neutralization breadth of the VRC13 and VRC16 antibodies can tolerate conservative changes to the epitope binding domain while still maintaining binding.

It is disclosed herein that the heavy chain CDRs of the VRC13 and VRC16 antibodies bind gp120. Camelid heavy chain antibodies exist as homodimers of a single heavy chain, dimerized via their constant regions (U.S. Pat. Nos. 5,840,526 and 6,838,254; and U.S. Patent Application Publication No. 2003-0088074). The variable domains of these camelid heavy chain antibodies, referred to as V_{H}H domains, retain the ability, when isolated as fragments of the V_{H} chain, to bind antigen with high specificity (Hamers-Casterman et al. Nature 363:446-448, 1993; Gahroudi et al. FEBS Lett. 414:521-526, 1997). The HCDRs disclosed herein can be included in a camelid antibody.

Antigen binding single V_{H} domains, called domain antibodies (dAb), have also been identified from a library of murine V_{H} genes amplified from genomic DNA of immunized mice (Ward et al. Nature 341:544-546, 1989). Human single immunoglobulin variable domain polypeptides capable of binding antigen with high affinity have also been described (see, for example, PCT Publication Nos. WO 2005/035572 and WO 2003/002609). The CDRs disclosed herein can also be included in a dAb.

CH2 and CH3 domain molecules are known in the art, see for example, PCT Publication No. 2009/099961, incorporated herein by reference. The CDRs disclosed herein can be include in a CH2 or CH3 domain molecule.

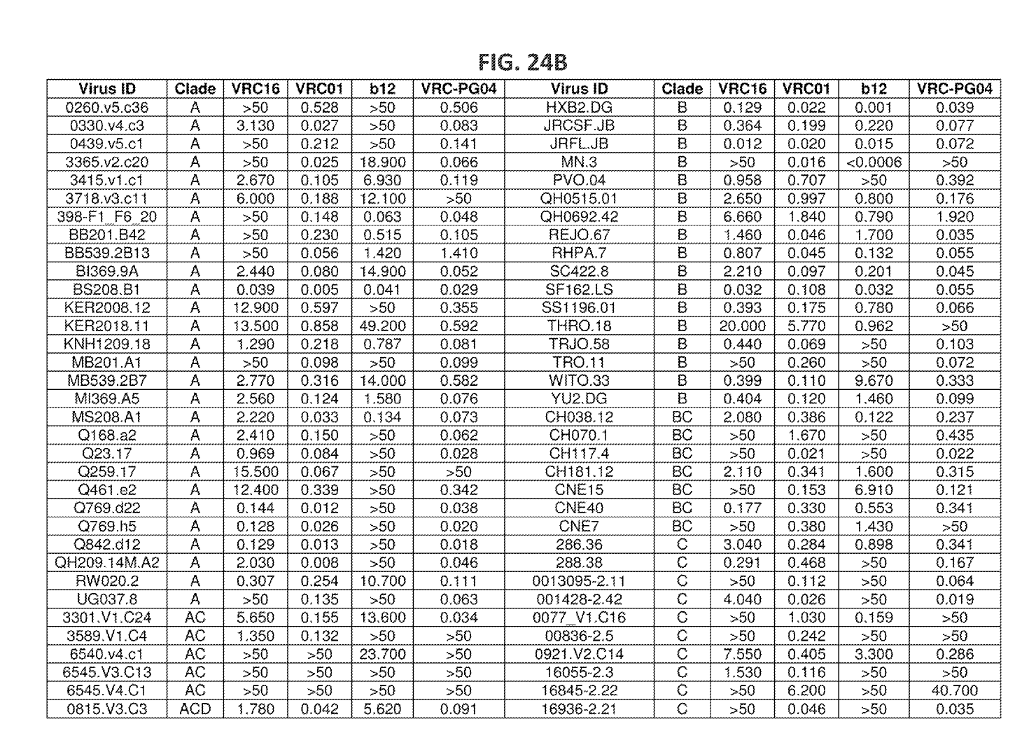

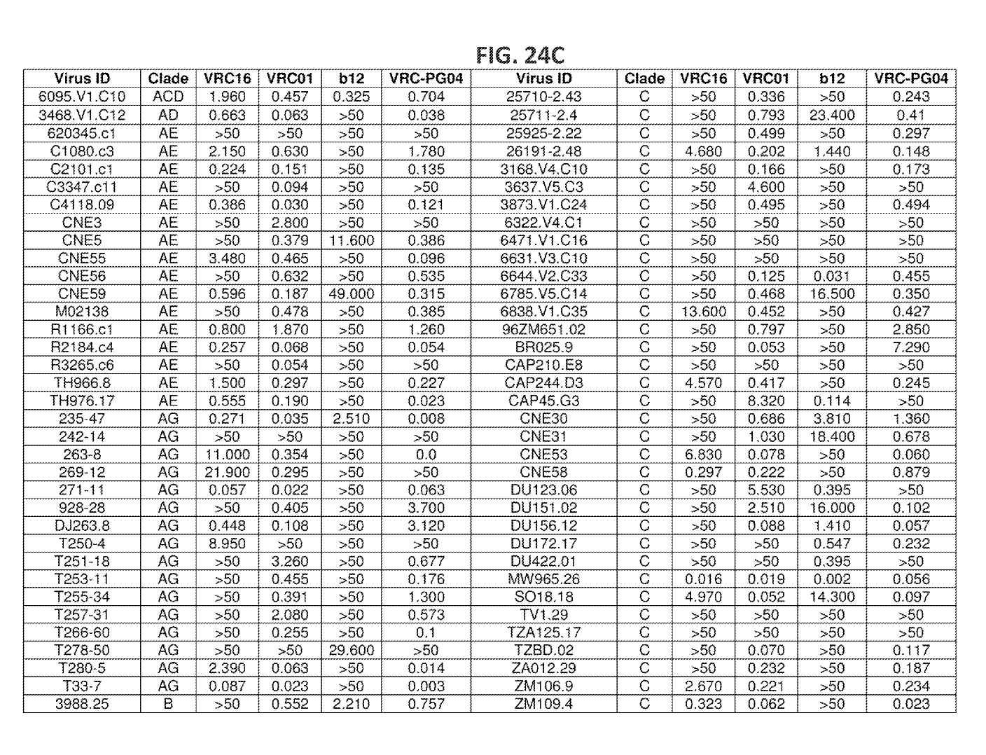

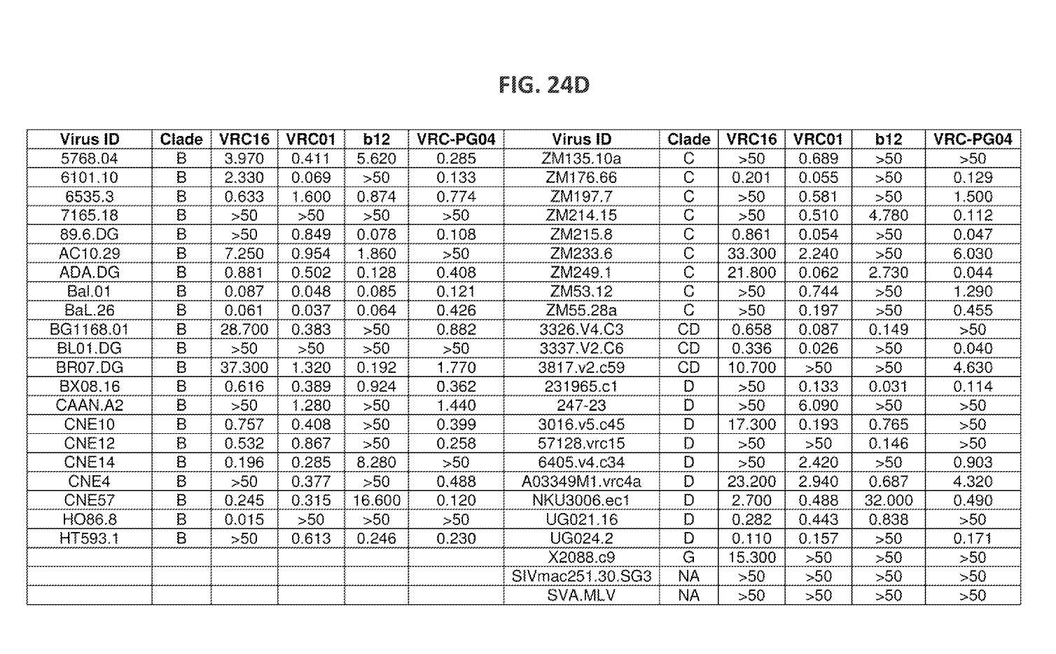

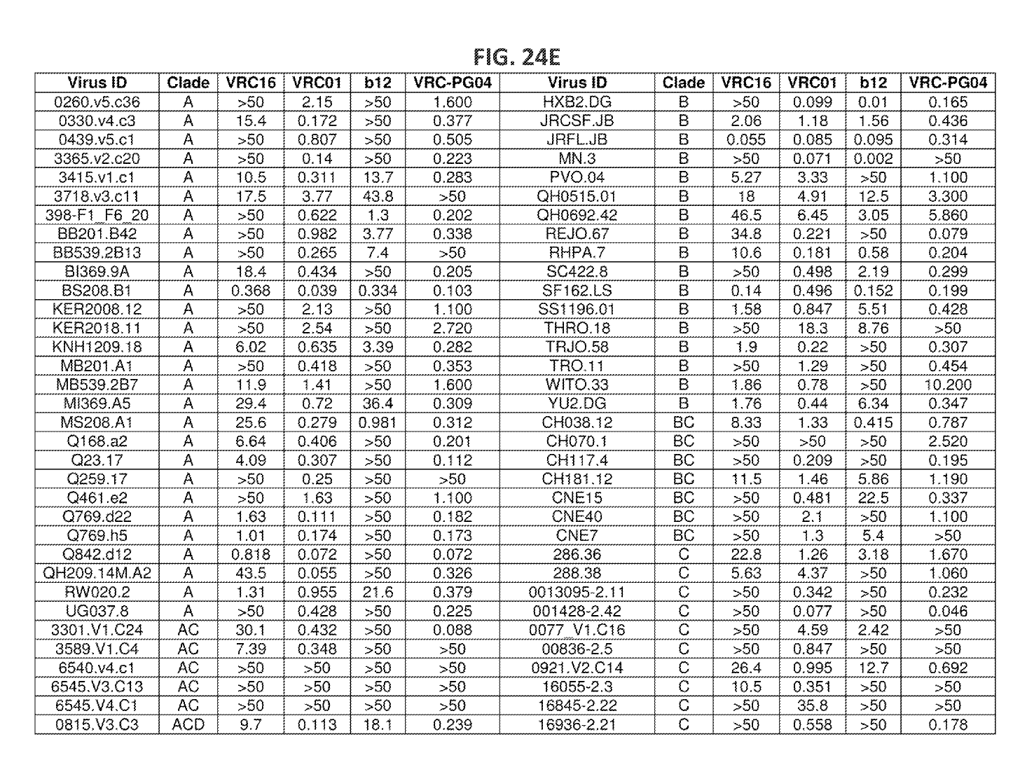

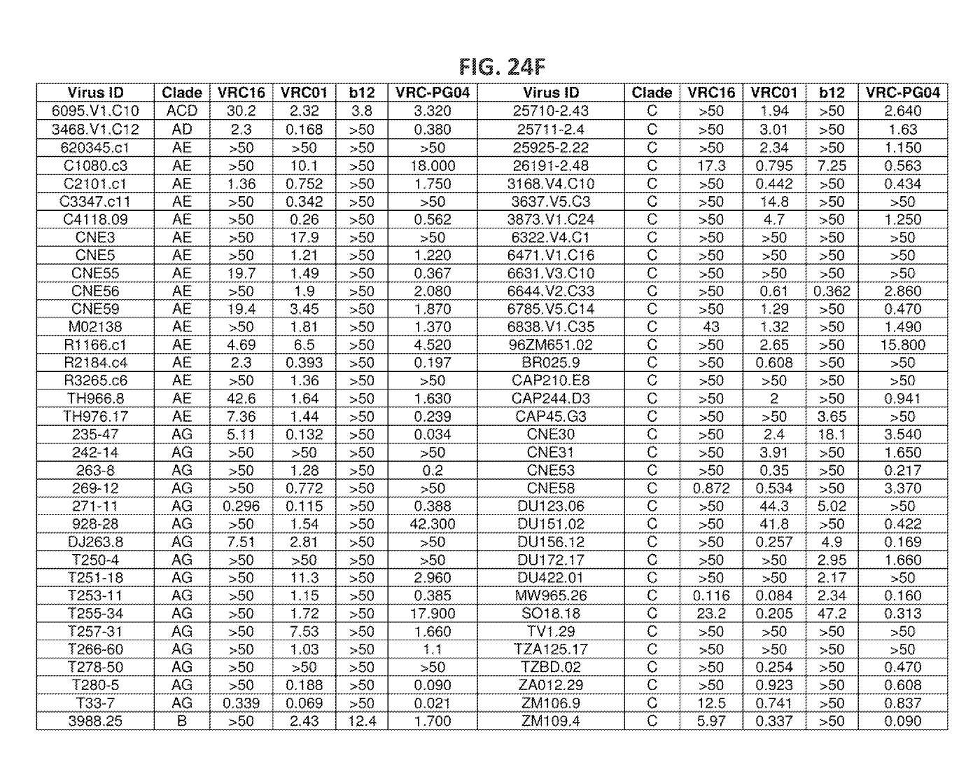

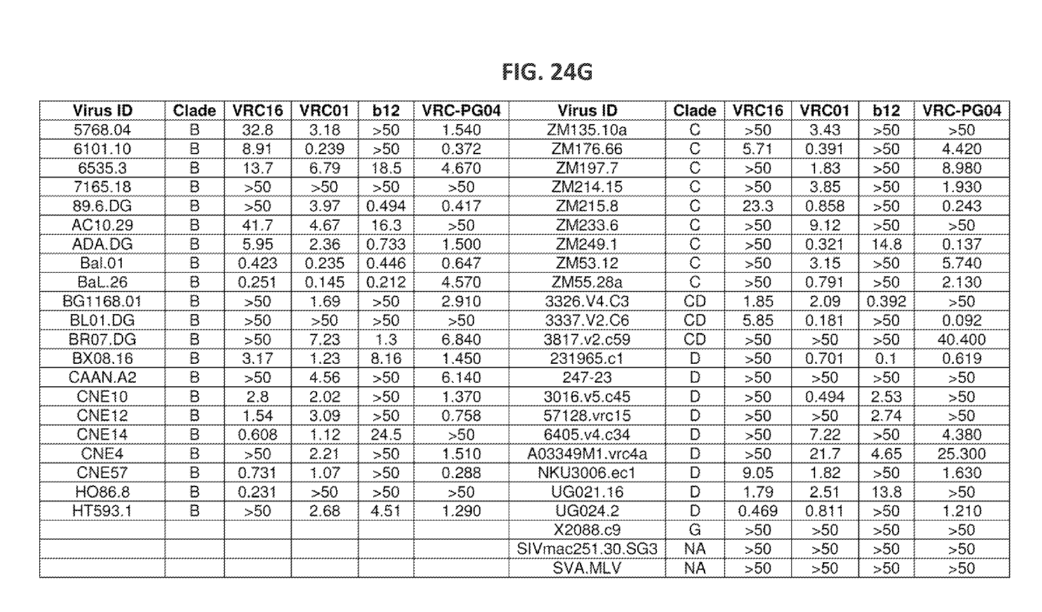

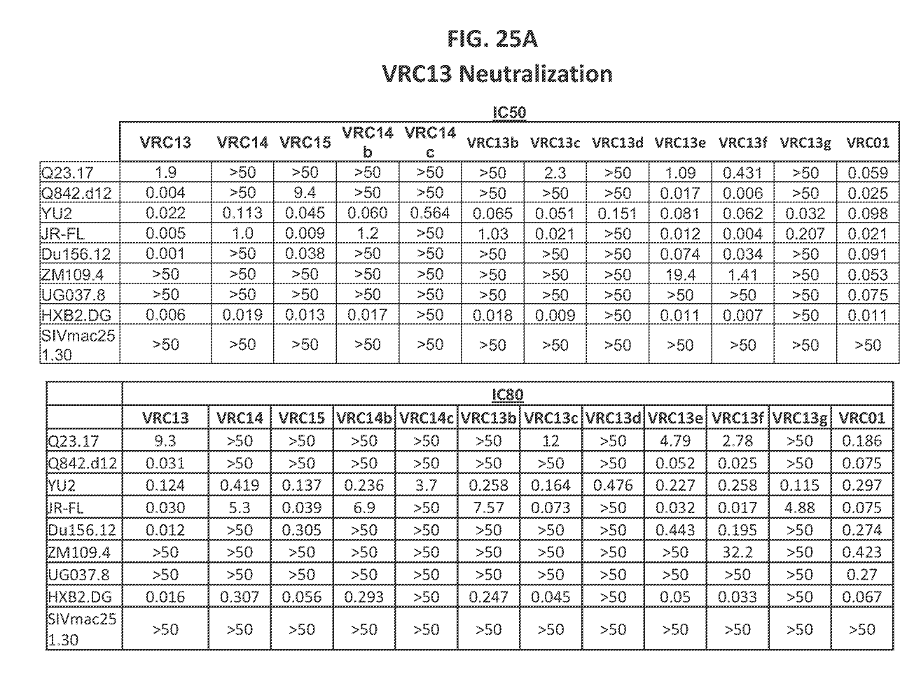

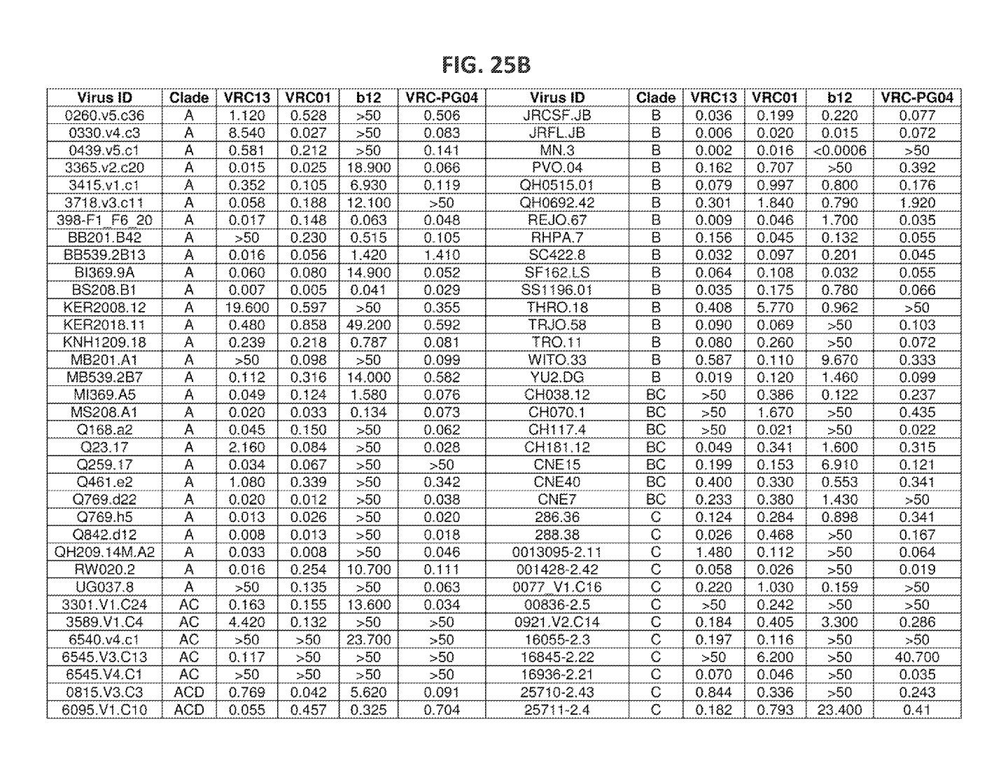

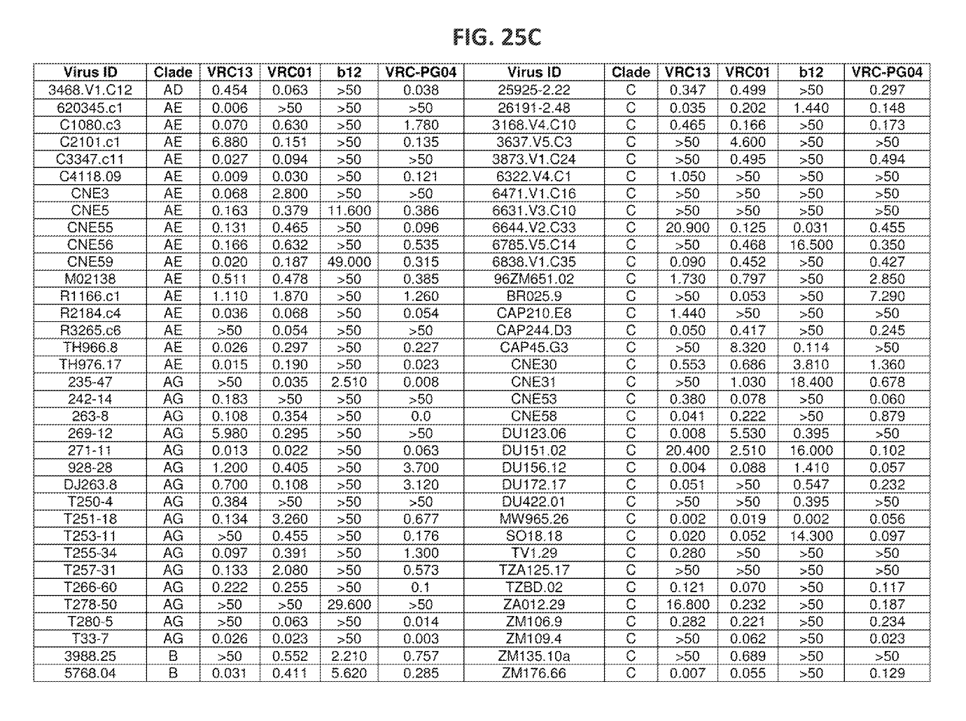

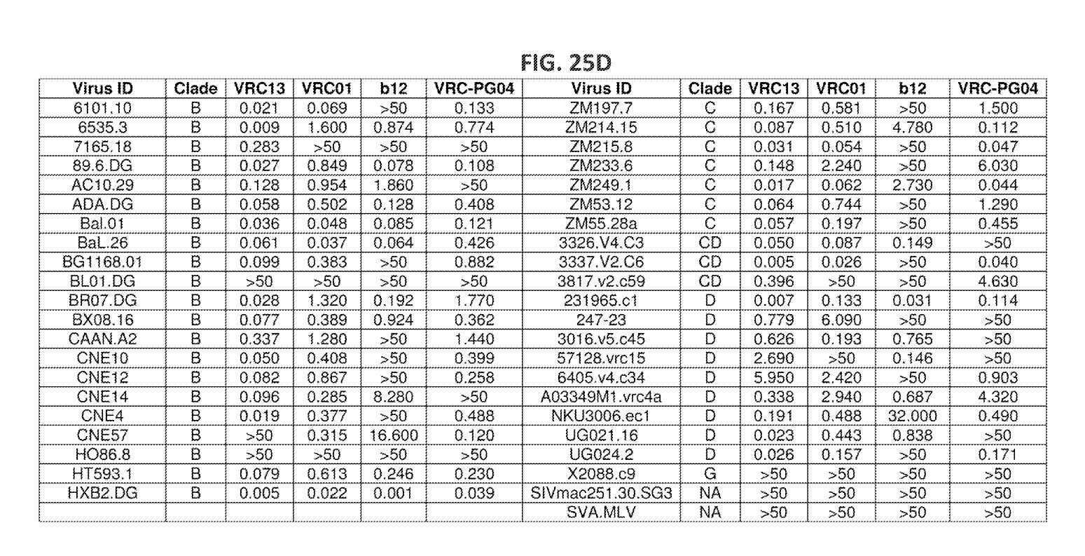

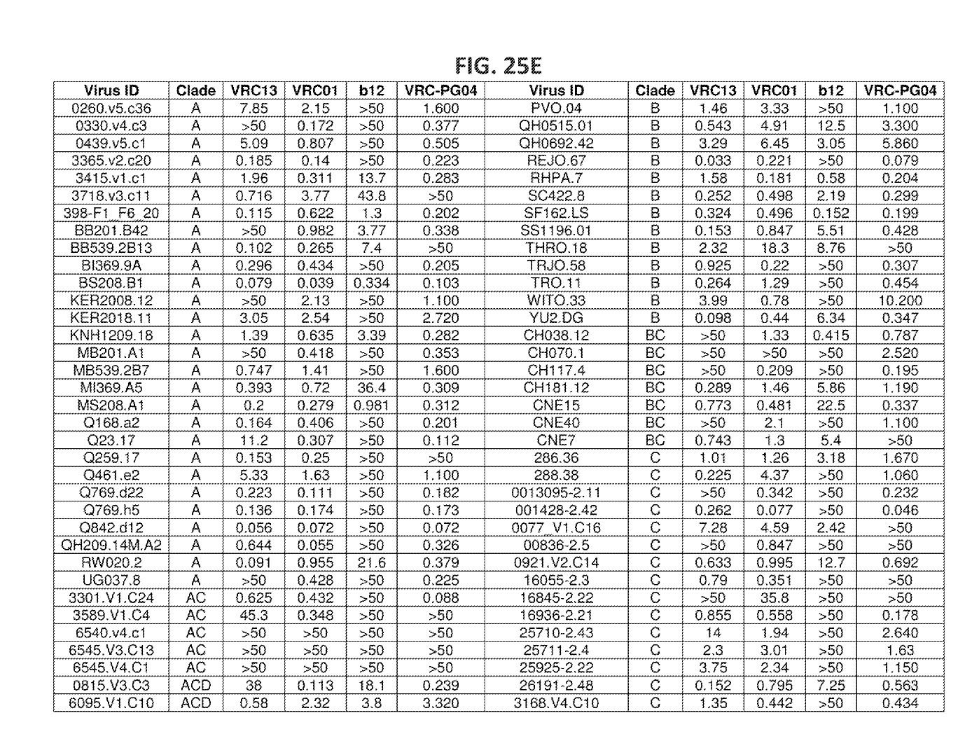

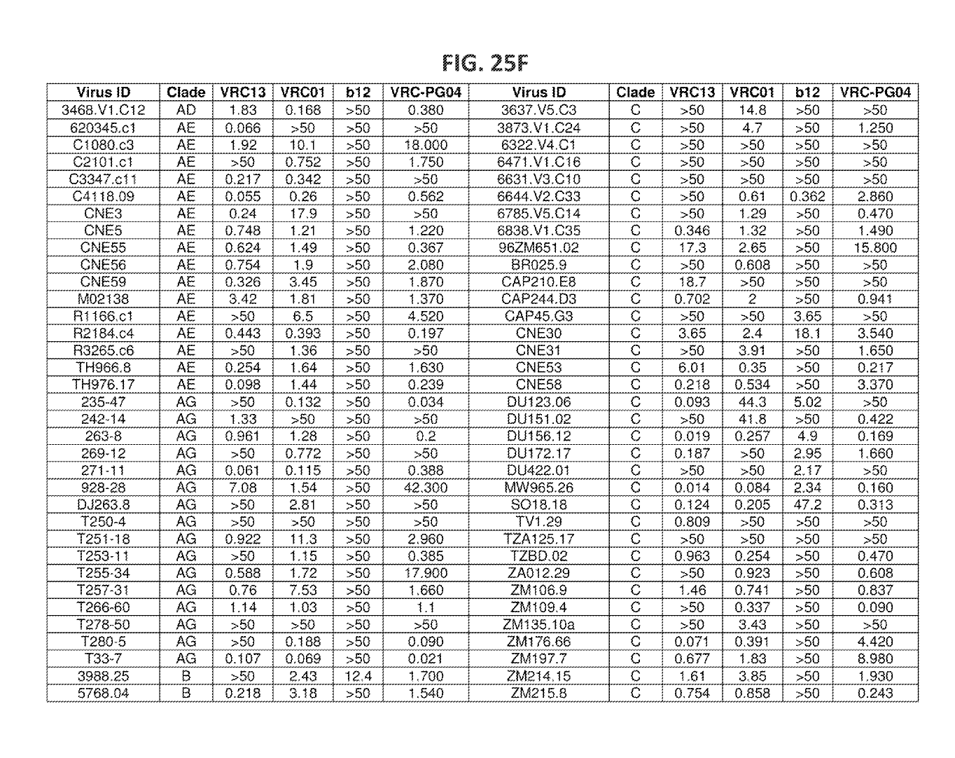

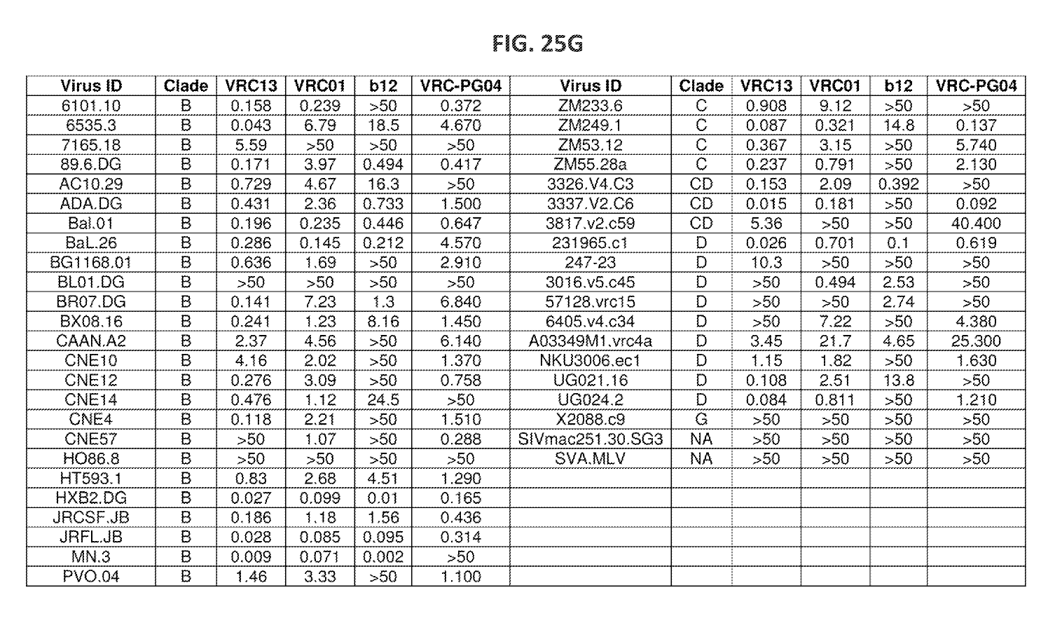

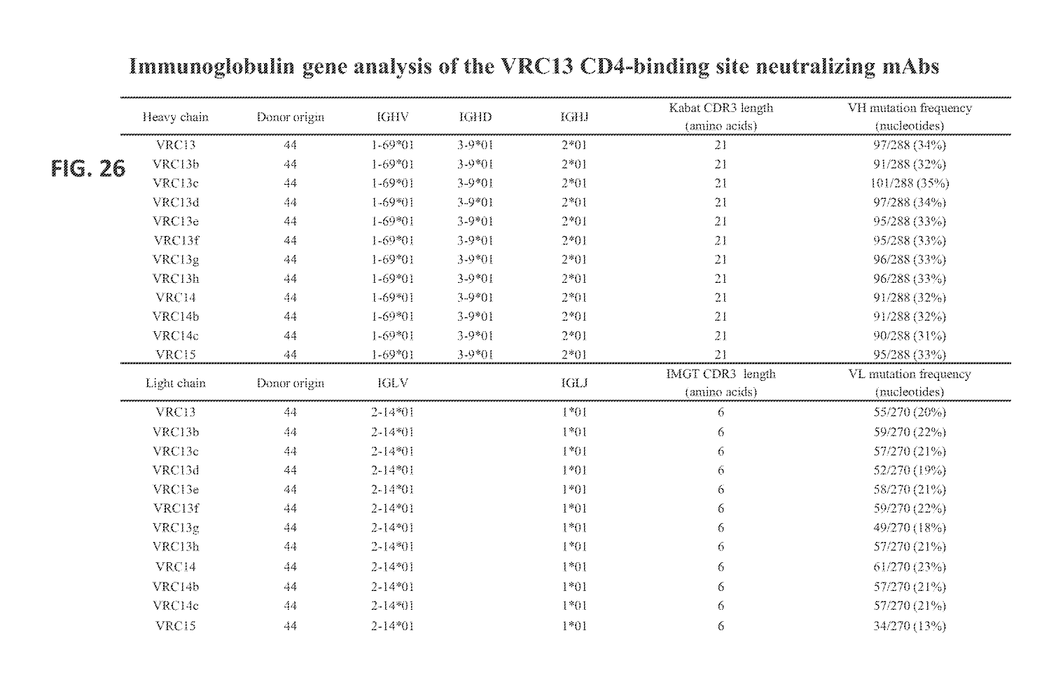

In some embodiments, the VRC13 antibodies can also be distinguished by neutralization breadth. In some embodiments, a VRC13 antibody can neutralize at least 80% (such as at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) of the HIV-1 isolates listed in FIGS. 25B-D with an IC50 of less than 50 μg/ml. In some embodiments, the VRC16 antibodies can also be distinguished by neutralization breadth. In some embodiments, a VRC16 antibody can neutralize at least 50% (such as at least 60%, at least 70%, at least 80%, at least 85%, at least 90%, at least 91%, at least 92%, at least 93%, at least 94%, at least 95%, at least 96%, at least 97%, at least 98% or at least 99%) of the HIV-1 isolates listed in FIGS. 24B-D with an IC50 of less than 50 μg/ml. The person of ordinary skill in the art if familiar with methods of measuring neutralization breadth and potency, for example such methods include the single-round HIV-1 Env-pseudoviruses infection of TZM-b1 cells (see, e.g., Li et al., J Virol 79, 10108-10125, 2005, incorporated by reference herein; see also, PCT Pub. No. WO2011/038290, incorporated by reference herein).

Fully human monoclonal antibodies include human framework regions. Thus, any of the antibodies that specifically bind gp120 herein can include the framework regions set forth in one of the VRC13 or VRC 16 antibodies disclosed herein. However, the framework regions can be from another source. Additional examples of framework sequences that can be used include the amino acid framework sequences of the heavy and light chains disclosed in PCT Publication No. WO 2006/074071 (see, for example, SEQ ID NOs: 1-16), which is herein incorporated by reference.

In some examples, the antibody can specifically bind gp120 with an affinity of at least about 1.5×10⁻⁸M, at least about 2.0×10⁻⁸M, at least about 2.5×10⁻⁸M, at least about 3.0×10⁻⁸M, at least about 3.5×10⁻⁸M, at least about 4.0×10⁻⁸M, at least about 4.5×10⁻⁸M, at least about 5.0×10⁻⁸ M or at least about 1.0×10⁻⁹M.

In some embodiments, an isolated antibody that specifically binds gp120 as disclosed herein includes up to 10 amino acid substitutions (such as up to 1, 2, 3, 4, 5, 6, 7, 8, or up to 9 amino acid substitutions) in the framework regions of the heavy chain of the antibody, or the light chain of the antibody, or the heavy and light chains of the antibody.

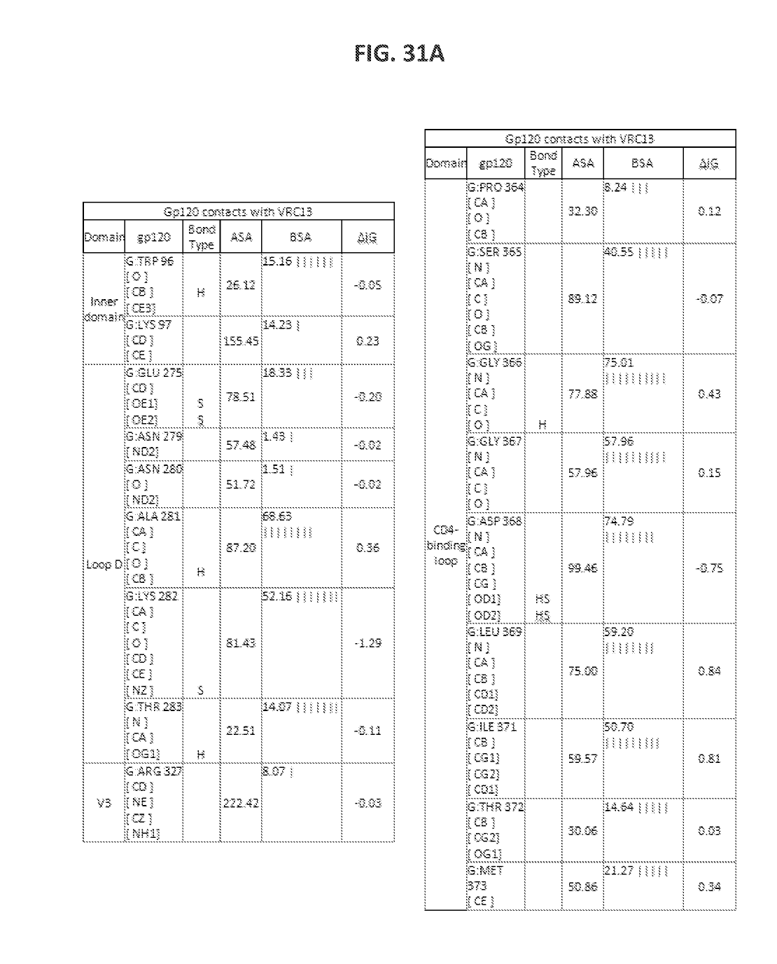

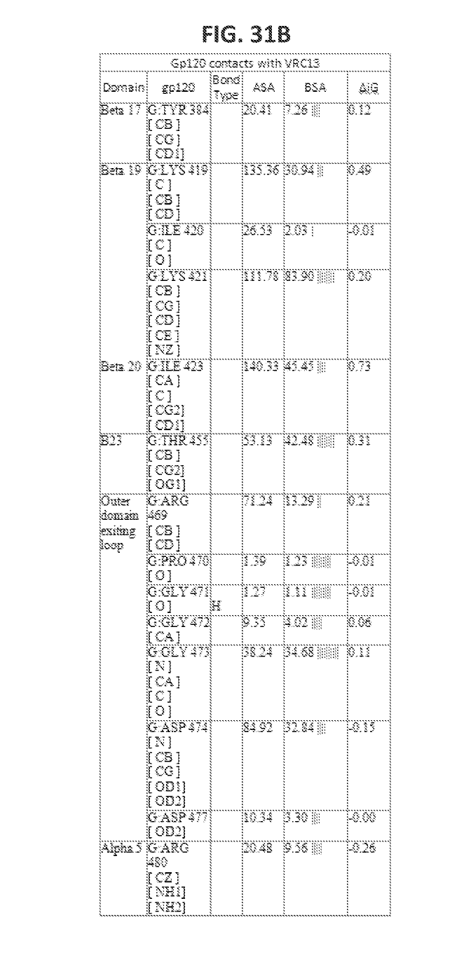

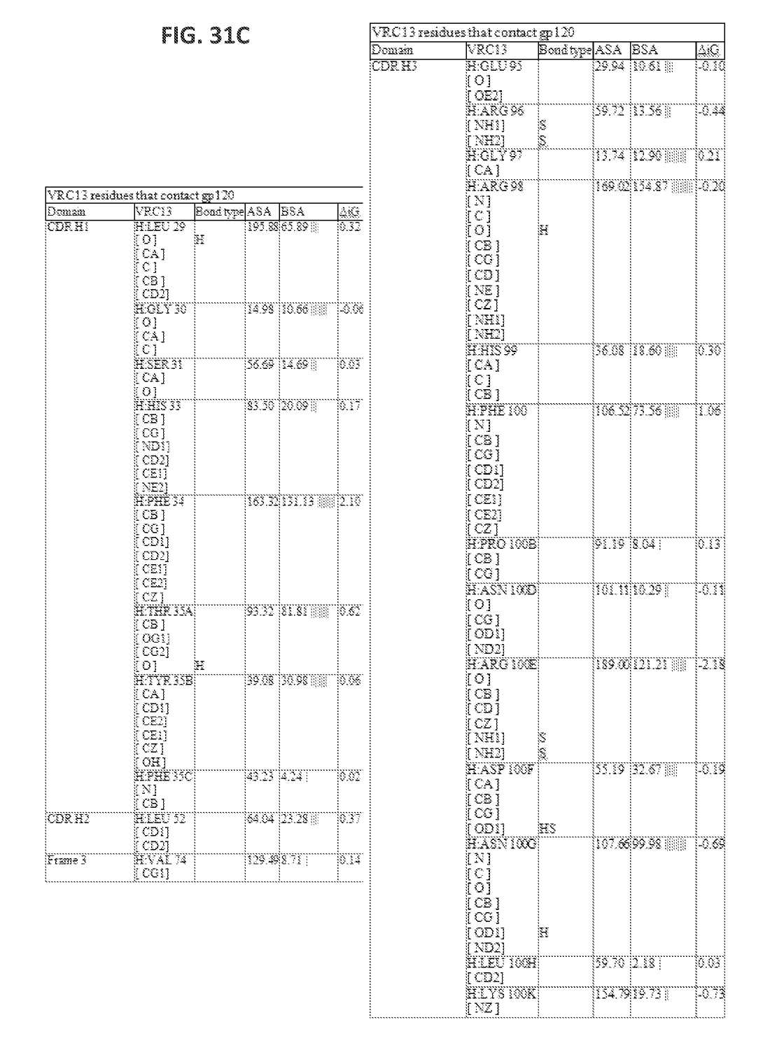

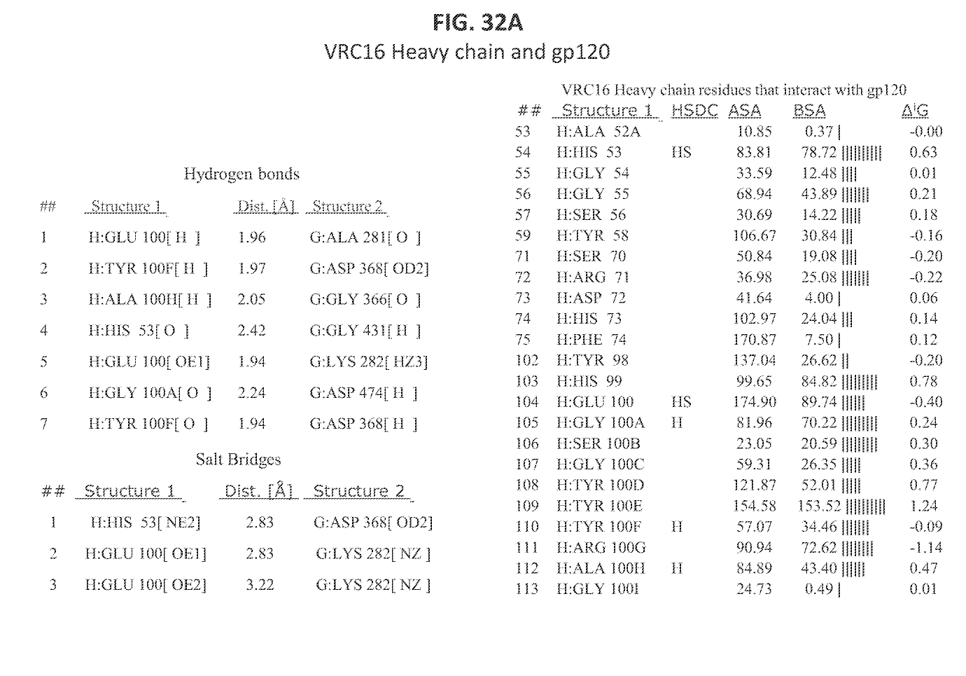

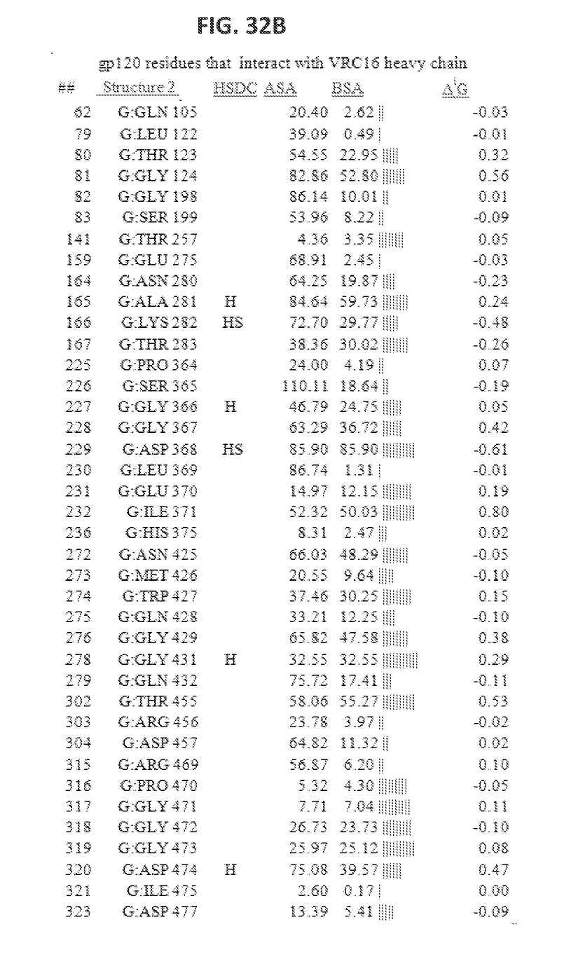

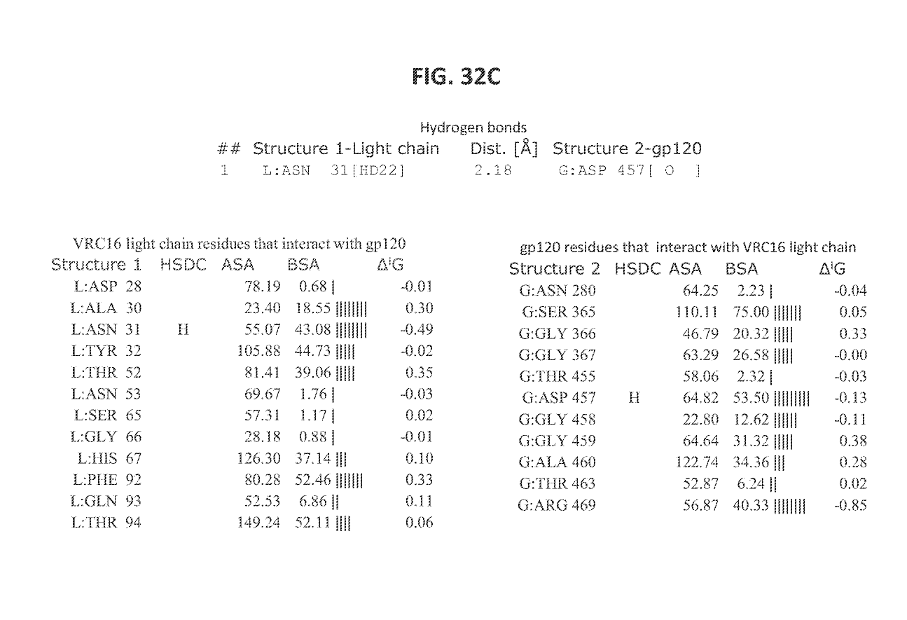

In some embodiments, one or more of the heavy and/or light chain complementarity determining regions (CDRs) from a VRC13 or VRC16 specific antibody is expressed on the surface of another protein, such as a scaffold protein. The expression of domains of antibodies on the surface of a scaffolding protein are known in the art (see e.g. Liu et al., J. Virology 85(17): 8467-8476, 2011). Such expression creates a chimeric protein that retains the binding for gp120. As shown in FIGS. 31 and 32, the VRC13 and VRC16 antibodies make most of their contacts through the heavy chain CDRs, such as CDR3. Thus, in some specific embodiments, one or more of the heavy chain CDRs is grafted onto a scaffold protein, such as one or more of heavy chain CDR1, CDR2, and/or CDR3. One or more CDRs can also be included in a diabody or another type of single chain antibody molecule.

The monoclonal antibody can be of any isotype. The monoclonal antibody can be, for example, an IgM or an IgG antibody, such as IgG₁, IgG₂, IgG₃, or IgG₄. The class of an antibody that specifically binds gp120 can be switched with another. In one aspect, a nucleic acid molecule encoding V_{L} or V_{H} is isolated using methods well-known in the art, such that it does not include any nucleic acid sequences encoding the constant region of the light or heavy chain, respectively. In particular examples, the V_{H} amino acid sequence is set forth as one of SEQ ID NO: 1, or 5-16. In other examples, the V_{L} amino acid sequence is set forth as one of SEQ ID NO: 2 or 17-28. In other examples, the V_{H} amino acid sequence is set forth as one of SEQ ID NO: 3, or 29-32. In other examples, the V_{L} amino acid sequence is set forth as one of SEQ ID NO: 4 or 33-36. A nucleic acid molecule encoding V_{L} or V_{H} is then operatively linked to a nucleic acid sequence encoding a C_{L} or C_{H} from a different class of immunoglobulin molecule. This can be achieved using a vector or nucleic acid molecule that comprises a C_{L} or C_{H} chain, as known in the art. For example, an antibody that specifically binds gp120, that was originally IgM may be class switched to an IgG. Class switching can be used to convert one IgG subclass to another, such as from IgG₁ to IgG₂, IgG₃, or IgG₄.

In some examples, the disclosed antibodies are oligomers of antibodies, such as dimers, trimers, tetramers, pentamers, hexamers, septamers, octomers and so on. In some examples, the antibodies are pentamers.

In several embodiments, the constant region of the antibody includes one or more amino acid substitutions to optimize in vivo half-life of the antibody. The serum half-life of IgG Abs is regulated by the neonatal Fc receptor (FcRn). Thus, in several embodiments, the antibody includes an amino acid substitution that increases binding to the FcRn. Several such substitutions are known to the person of ordinary skill in the art, such as substitutions at IgG constant regions T250Q and M428L (see, e.g., Hinton et al., J Immunol., 176:346-356, 2006); M428L and N434S (see, e.g., Zalevsky, et al., Nature Biotechnology, 28:157-159, 2010); N434A (see, e.g., Petkova et al., Int. Immunol., 18:1759-1769, 2006); T307A, E380A, and N434A (see, e.g., Petkova et al., Int. Immunol., 18:1759-1769, 2006); and M252Y, S254T, and T256E (see, e.g., Dall′Acqua et al., J. Biol. Chem., 281:23514-23524, 2006).

In some embodiments, the constant region of the antibody includes one of more amino acid substitutions to optimize Antibody-dependent cell-mediated cytotoxicity (ADCC). ADCC is mediated primarily through a set of closely related Fcγ receptors. In some embodiments, the antibody includes one or more amino acid substitutions that increase binding to FcγRIIIa. Several such substitutions are known to the person of ordinary skill in the art, such as substitutions at IgG constant regions S239D and I332E (see, e.g., Lazar et al., Proc. Natl., Acad. Sci. U.S.A., 103:4005-4010, 2006); and S239D, A330L, and I332E (see, e.g., Lazar et al., Proc. Natl., Acad. Sci. U.S.A., 103:4005-4010, 2006).

Combinations of the above substitutions are also included, to generate an IgG constant region with increased binding to FcRn and FcγRIIIa. The combinations increase antibody half-life and ADCC. For example, such combination include antibodies with the following amino acid substitution in the Fc region:

(1) S239D/I332E and T250Q/M428L;

(2) S239D/I332E and M428L/N434S;

(3) S239D/I332E and N434A;

(4) S239D/I332E and T307A/E380A/N434A;

(5) S239D/I332E and M252Y/S254T/T256E;

(6) S239D/A330L/I332E and T250Q/M428L;

(7) S239D/A330L/I332E and M428L/N434S;

(8) S239D/A330L/I332E and N434A;

(9) S239D/A330L/I332E and T307A/E380A/N434A; or

(10) S239D/A330L/I332E and M252Y/S254T/T256E.

In some examples, the antibodies, or an antigen binding fragment thereof is modified such that it is directly cytotoxic to infected cells, or uses natural defenses such as complement, antibody dependent cellular cytotoxicity (ADCC), or phagocytosis by macrophages.

Antibody fragments are encompassed by the present disclosure, such as Fab, F(ab′)₂, and Fv which include a heavy chain and light chain variable region and specifically bind gp120. These antibody fragments retain the ability to selectively bind with the antigen and are “antigen-binding” fragments. These fragments include:

(1) Fab, the fragment which contains a monovalent antigen-binding fragment of an antibody molecule, can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;

(4) Fv, a genetically engineered fragment containing the variable region of the light chain and the variable region of the heavy chain expressed as two chains; and

(5) Single chain antibody (such as scFv), defined as a genetically engineered molecule containing the variable region of the light chain, the variable region of the heavy chain, linked by a suitable polypeptide linker as a genetically fused single chain molecule.

(6) A dimer of a single chain antibody (scFV₂), defined as a dimer of a scFV. This has also been termed a “miniantibody.”

Methods of making these fragments are known in the art (see for example, Harlow and Lane, Antibodies: A Laboratory Manual, Cold Spring Harbor Laboratory, New York, 1988).

In a further group of embodiments, the antibodies are Fv antibodies, which are typically about 25 kDa and contain a complete antigen-binding site with three CDRs per each heavy chain and each light chain. To produce these antibodies, the V_{H} and the V_{L} can be expressed from two individual nucleic acid constructs in a host cell. In particular examples, the V_{H} amino acid sequence includes the CDRs from one of SEQ ID NOs: 1, 3, 6-16 or 29-32. In other examples, the V_{L} amino acid sequence includes the CDRs from one of SEQ ID NOs: 2, 4, 17-28 or 33-36. In additional examples, the V_{H} amino acid sequence includes the amino acid sequence set forth as one of SEQ ID NOs: 1, 3, or 5-16 or 29-32or encoded by any one of SEQ ID NOs: 35-115. In other examples, the V_{L} amino acid sequence includes the amino acid sequence set forth as SEQ ID NOs: 2, 4, 17-28 or 33-36. In further embodiments, the V_{H} amino acid sequence includes the CDRs from one of SEQ ID NOs: 1 or 5-16 and the V_{L} amino acid sequence includes the CDRs from one of SEQ ID NOs: 2, or 17-28. In more embodiments, the V_{H} amino acid sequence includes the CDRs from one of SEQ ID NOs: 3 or 29-32 and the V_{L} amino acid sequence includes the CDRs from one of SEQ ID NOs: 4, or 33-36.

If the V_{H} and the V_{L} are expressed non-contiguously, the chains of the Fv antibody are typically held together by noncovalent interactions. However, these chains tend to dissociate upon dilution, so methods have been developed to crosslink the chains through glutaraldehyde, intermolecular disulfides, or a peptide linker. Thus, in one example, the Fv can be a disulfide stabilized Fv (dsFv), wherein the heavy chain variable region and the light chain variable region are chemically linked by disulfide bonds.

In an additional example, the Fv fragments comprise V_{H} and V_{L} chains connected by a peptide linker. These single-chain antigen binding proteins (scFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing scFvs are known in the art (see Whitlow et al., Methods: a Companion to Methods in Enzymology, Vol. 2, page 97, 1991; Bird et al., Science 242:423, 1988; U.S. Pat. No. 4,946,778; Pack et al., Bio/Technology 11:1271, 1993; and Sandhu, supra). Dimers of a single chain antibody (scFV₂), are also contemplated.

Antibody fragments can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see U.S. Pat. No. 4,036,945 and U.S. Pat. No. 4,331,647, and references contained therein; Nisonhoff et al., Arch. Biochem. Biophys. 89:230, 1960; Porter, Biochem. J. 73:119, 1959; Edelman et al., Methods in Enzymology, Vol. 1, page 422, Academic Press, 1967; and Coligan et al. at sections 2.8.1-2.8.10 and 2.10.1-2.10.4).

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

One of skill will realize that conservative variants of the antibodies can be produced. Such conservative variants employed in antibody fragments, such as dsFv fragments or in scFv fragments, will retain critical amino acid residues necessary for correct folding and stabilizing between the V_{H} and the V_{L} regions, and will retain the charge characteristics of the residues in order to preserve the low pI and low toxicity of the molecules. Amino acid substitutions (such as at most one, at most two, at most three, at most four, or at most five amino acid substitutions) can be made in the V_{H} and the V_{L} regions to increase yield. In particular examples, the V_{H} sequence is SEQ ID NOs: 1, 3, 5-16 or 29-32. In other examples, the V_{L} sequence is SEQ ID NOs: 2, 4, 7-28 OR 33-36. Conservative amino acid substitution tables providing functionally similar amino acids are well known to one of ordinary skill in the art. The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Variable light chain (V_{L}) and variable heavy chain (V_{H}) segments of the antibodies can be “mixed and matched”, in which different pairs of the V_{L} and V_{H} segments are screened for gp120 binding to select V_{L}/V_{H} pair combinations of interest. Additionally, to increase binding affinity of the antibody, the V_{L} and V_{H} segments can be randomly mutated, such as within H-CDR3 region or the L-CDR3 region, in a process analogous to the in vivo somatic mutation process responsible for affinity maturation of antibodies during a natural immune response. Thus in vitro affinity maturation can be accomplished by amplifying V_{H} and V_{L} regions using PCR primers complementary to the H-CDR3 or L-CDR3, respectively. In this process, the primers have been “spiked” with a random mixture of the four nucleotide bases at certain positions such that the resultant PCR products encode V_{H} and V_{L} segments into which random mutations have been introduced into the V_{H} and/or V_{L} CDR3 regions. These randomly mutated V_{H} and V_{L} segments can be tested to determine the binding affinity for gp120. In particular examples, the V_{H} amino acid sequence is one of SEQ ID NOs: 1, 3, 5-16 or 29-32. In other examples, the V_{L} amino acid sequence is SEQ ID NOs: 2, 4, 17-28 or 33-36.

Effector molecules, such as therapeutic, diagnostic, or detection moieties can be linked to an antibody of interest, using any number of means known to those of skill in the art. Both covalent and noncovalent attachment means may be used. The procedure for attaching an effector molecule to an antibody varies according to the chemical structure of the effector. Polypeptides typically contain a variety of functional groups; such as carboxylic acid (COOH), free amine (—NH₂) or sulfhydryl (—SH) groups, which are available for reaction with a suitable functional group on an antibody to result in the binding of the effector molecule. Alternatively, the antibody is derivatized to expose or attach additional reactive functional groups. The derivatization may involve attachment of any of a number of linker molecules such as those available from Pierce Chemical Company, Rockford, Ill. The linker can be any molecule used to join the antibody to the effector molecule. The linker is capable of forming covalent bonds to both the antibody and to the effector molecule. Suitable linkers are well known to those of skill in the art and include, but are not limited to, straight or branched-chain carbon linkers, heterocyclic carbon linkers, or peptide linkers. Where the antibody and the effector molecule are polypeptides, the linkers may be joined to the constituent amino acids through their side groups (such as through a disulfide linkage to cysteine) or to the alpha carbon amino and carboxyl groups of the terminal amino acids.

In some circumstances, it is desirable to free the effector molecule from the antibody when the immunoconjugate has reached its target site. Therefore, in these circumstances, immunoconjugates will comprise linkages that are cleavable in the vicinity of the target site. Cleavage of the linker to release the effector molecule from the antibody may be prompted by enzymatic activity or conditions to which the immunoconjugate is subjected either inside the target cell or in the vicinity of the target site.

In view of the large number of methods that have been reported for attaching a variety of radiodiagnostic compounds, radiotherapeutic compounds, label (such as enzymes or fluorescent molecules) drugs, toxins, and other agents to antibodies one skilled in the art will be able to determine a suitable method for attaching a given agent to an antibody or other polypeptide.

The antibodies or antibody fragments disclosed herein can be derivatized or linked to another molecule (such as another peptide or protein). In general, the antibody or portion thereof is derivatized such that the binding to gp120 is not affected adversely by the derivatization or labeling. For example, the antibody can be functionally linked (by chemical coupling, genetic fusion, noncovalent association or otherwise) to one or more other molecular entities, such as another antibody (for example, a bispecific antibody or a diabody), a detection agent, a pharmaceutical agent, and/or a protein or peptide that can mediate associate of the antibody or antibody portion with another molecule (such as a streptavidin core region or a polyhistidine tag).

One type of derivatized antibody is produced by cross-linking two or more antibodies (of the same type or of different types, such as to create bispecific antibodies). Suitable crosslinkers include those that are heterobifunctional, having two distinctly reactive groups separated by an appropriate spacer (such as m-maleimidobenzoyl-N-hydroxysuccinimide ester) or homobifunctional (such as disuccinimidyl suberate). Such linkers are available from Pierce Chemical Company (Rockford, Ill.).

An antibody that specifically binds gp120 can be labeled with a detectable moiety. Useful detection agents include fluorescent compounds, including fluorescein, fluorescein isothiocyanate, rhodamine, 5-dimethylamine-l-napthalenesulfonyl chloride, phycoerythrin, lanthanide phosphors and the like. Bioluminescent markers are also of use, such as luciferase, Green fluorescent protein, Yellow fluorescent protein. An antibody can also be labeled with enzymes that are useful for detection, such as horseradish peroxidase, β-galactosidase, luciferase, alkaline phosphatase, glucose oxidase and the like. When an antibody is labeled with a detectable enzyme, it can be detected by adding additional reagents that the enzyme uses to produce a reaction product that can be discerned. For example, when the agent horseradish peroxidase is present the addition of hydrogen peroxide and diaminobenzidine leads to a colored reaction product, which is visually detectable. An antibody may also be labeled with biotin, and detected through indirect measurement of avidin or streptavidin binding. It should be noted that the avidin itself can be labeled with an enzyme or a fluorescent label.

An antibody may be labeled with a magnetic agent, such as gadolinium. Antibodies can also be labeled with lanthanides (such as europium and dysprosium), and manganese. Paramagnetic particles such as superparamagnetic iron oxide are also of use as labels. An antibody may also be labeled with a predetermined polypeptide epitopes recognized by a secondary reporter (such as leucine zipper pair sequences, binding sites for secondary antibodies, metal binding domains, epitope tags). In some embodiments, labels are attached by spacer arms of various lengths to reduce potential steric hindrance.

An antibody can also be labeled with a radiolabeled amino acid. The radiolabel may be used for both diagnostic and therapeutic purposes. Examples of labels for polypeptides include, but are not limited to, the following radioisotopes or radionucleotides: ³H, ¹⁴C, ¹⁵N, ³⁵S, ⁹⁰Y, ⁹⁹Tc, ¹¹¹In, ¹²⁵I, ¹³¹I

An antibody can also be derivatized with a chemical group such as polyethylene glycol (PEG), a methyl or ethyl group, or a carbohydrate group. These groups may be useful to improve the biological characteristics of the antibody, such as to increase serum half-life or to increase tissue binding.

Means of detecting such labels are well known to those of skill in the art. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

### B. Polynucleotides and Expression

Nucleic acid molecules (also referred to as polynucleotides) encoding the polypeptides provided herein (including, but not limited to antibodies) can readily be produced by one of skill in the art. For example, these nucleic acids can be produced using the amino acid sequences provided herein (such as the CDR sequences, heavy chain and light chain sequences).

One of skill in the art can readily use the genetic code to construct a variety of functionally equivalent nucleic acids, such as nucleic acids which differ in sequence but which encode the same antibody sequence, or encode a conjugate or fusion protein including the V_{L} and/or V_{H} nucleic acid sequence.

Nucleic acid sequences encoding the antibodies that specifically bind gp120 can be prepared by any suitable method including, for example, cloning of appropriate sequences or by direct chemical synthesis by methods such as the phosphotriester method of Narang et al., Meth. Enzymol. 68:90-99, 1979; the phosphodiester method of Brown et al., Meth. Enzymol. 68:109-151, 1979; the diethylphosphoramidite method of Beaucage et al., Tetra. Lett. 22:1859-1862, 1981; the solid phase phosphoramidite triester method described by Beaucage & Caruthers, Tetra. Letts. 22(20):1859-1862, 1981, for example, using an automated synthesizer as described in, for example, Needham-VanDevanter et al., Nucl. Acids Res. 12:6159-6168, 1984; and, the solid support method of U.S. Pat. No. 4,458,066. Chemical synthesis produces a single stranded oligonucleotide. This can be converted into double stranded DNA by hybridization with a complementary sequence or by polymerization with a DNA polymerase using the single strand as a template. One of skill would recognize that while chemical synthesis of DNA is generally limited to sequences of about 100 bases, longer sequences may be obtained by the ligation of shorter sequences.

Exemplary nucleic acids can be prepared by cloning techniques. Examples of appropriate cloning and sequencing techniques, and instructions sufficient to direct persons of skill through many cloning exercises are found in Sambrook et al., supra, Berger and Kimmel (eds.), supra, and Ausubel, supra. Product information from manufacturers of biological reagents and experimental equipment also provide useful information. Such manufacturers include the SIGMA Chemical Company (Saint Louis, Mo.), R&D Systems (Minneapolis, Minn.), Pharmacia Amersham (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), Invitrogen (Carlsbad, Calif.), and Applied Biosystems (Foster City, Calif.), as well as many other commercial sources known to one of skill.

Nucleic acids can also be prepared by amplification methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill

Any of the nucleic acids encoding any of the antibodies, V_{H} and/or V_{L}, disclosed herein (or fragment thereof) can be expressed in a recombinantly engineered cell such as bacteria, plant, yeast, insect and mammalian cells. These antibodies can be expressed as individual V_{H} and/or V_{L} chain, or can be expressed as a fusion protein. An immunoadhesin can also be expressed. Thus, in some examples, nucleic acids encoding a V_{H} and V_{L}, and immunoadhesin are provided. The nucleic acid sequences can optionally encode a leader sequence.

To create a single chain antibody, (scFv) the V_{H}- and V_{L}-encoding DNA fragments are operatively linked to another fragment encoding a flexible linker, e.g., encoding the amino acid sequence (Gly₄-Ser)₃, such that the V_{H} and V_{L} sequences can be expressed as a contiguous single-chain protein, with the V_{L} and V_{H} domains joined by the flexible linker (see, e.g., Bird et al., Science 242:423-426, 1988; Huston et al., Proc. Natl. Acad. Sci. USA 85:5879-5883, 1988; McCafferty et al., Nature 348:552-554, 1990). Optionally, a cleavage site can be included in a linker, such as a furin cleavage site.

The nucleic acid encoding the V_{H} and/or the V_{L} optionally can encode an Fc domain (immunoadhesin). The Fc domain can be an IgA, IgM or IgG Fc domain. The Fc domain can be an optimized Fc domain, as described in U.S. Published Patent Application No. 20100/093979, incorporated herein by reference. In one example, the immunoadhesin is an IgG₁ Fc. In one example, the immunoadhesin is an IgG₃ Fc.

The single chain antibody may be monovalent, if only a single V_{H} and V_{L} are used, bivalent, if two V_{H} and V_{L} are used, or polyvalent, if more than two V_{H} and V_{L} are used. Bispecific or polyvalent antibodies may be generated that bind specifically to gp120 and to another molecule, such as gp41. The encoded V_{H} and V_{L} optionally can include a furin cleavage site between the V_{H} and V_{L} domains.

It is expected that those of skill in the art are knowledgeable in the numerous expression systems available for expression of proteins including E. coli, other bacterial hosts, yeast, and various higher eukaryotic cells such as the COS, CHO, HeLa and myeloma cell lines.

The host cell can be a gram positive bacteria including, but are not limited to, Bacillus, Streptococcus, Streptomyces, Staphylococcus, Enterococcus, Lactobacillus, Lactococcus, Clostridium, Geobacillus, and Oceanobacillus. Methods for expressing protein in gram positive bacteria, such as Lactobaccillus are well known in the art, see for example, U.S. Published Patent Application No. 20100/080774. Expression vectors for lactobacillus are described, for example in U.S. Pat. No. 6,100,388, and U.S. Pat. No. 5,728,571. Leader sequences can be included for expression in Lactobacillus. Gram negative bacteria include, but not limited to, E. coli, Pseudomonas, Salmonella, Campylobacter, Helicobacter, Flavobacterium, Fusobacterium, Ilyobacter, Neisseria, and Ureaplasma.

One or more DNA sequences encoding the antibody or fragment thereof can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art. Hybridomas expressing the antibodies of interest are also encompassed by this disclosure.

The expression of nucleic acids encoding the isolated proteins described herein can be achieved by operably linking the DNA or cDNA to a promoter (which is either constitutive or inducible), followed by incorporation into an expression cassette. The promoter can be any promoter of interest, including a cytomegalovirus promoter and a human T cell lymphotrophic virus promoter (HTLV)-1. Optionally, an enhancer, such as a cytomegalovirus enhancer, is included in the construct. The cassettes can be suitable for replication and integration in either prokaryotes or eukaryotes. Typical expression cassettes contain specific sequences useful for regulation of the expression of the DNA encoding the protein. For example, the expression cassettes can include appropriate promoters, enhancers, transcription and translation terminators, initiation sequences, a start codon (i.e., ATG) in front of a protein-encoding gene, splicing signal for introns, sequences for the maintenance of the correct reading frame of that gene to permit proper translation of mRNA, and stop codons. The vector can encode a selectable marker, such as a marker encoding drug resistance (for example, ampicillin or tetracycline resistance).

To obtain high level expression of a cloned gene, it is desirable to construct expression cassettes which contain, at the minimum, a strong promoter to direct transcription, a ribosome binding site for translational initiation (internal ribosomal binding sequences), and a transcription/translation terminator. For E. coli, this includes a promoter such as the T7, trp, lac, or lambda promoters, a ribosome binding site, and preferably a transcription termination signal. For eukaryotic cells, the control sequences can include a promoter and/or an enhancer derived from, for example, an immunoglobulin gene, HTLV, SV40 or cytomegalovirus, and a polyadenylation sequence, and can further include splice donor and/or acceptor sequences (for example, CMV and/or HTLV splice acceptor and donor sequences). The cassettes can be transferred into the chosen host cell by well-known methods such as transformation or electroporation for E. coli and calcium phosphate treatment, electroporation or lipofection for mammalian cells. Cells transformed by the cassettes can be selected by resistance to antibiotics conferred by genes contained in the cassettes, such as the amp, gpt, neo and hyg genes.

When the host is a eukaryote, such methods of transfection of DNA as calcium phosphate coprecipitates, conventional mechanical procedures such as microinjection, electroporation, insertion of a plasmid encased in liposomes, or virus vectors may be used. Eukaryotic cells can also be cotransformed with polynucleotide sequences encoding the antibody, labeled antibody, or antigen binding fragment thereof, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982). One of skill in the art can readily use an expression systems such as plasmids and vectors of use in producing proteins in cells including higher eukaryotic cells such as the COS, CHO, HeLa and myeloma cell lines.

Modifications can be made to a nucleic acid encoding a polypeptide described herein without diminishing its biological activity. Some modifications can be made to facilitate the cloning, expression, or incorporation of the targeting molecule into a fusion protein. Such modifications are well known to those of skill in the art and include, for example, termination codons, a methionine added at the amino terminus to provide an initiation, site, additional amino acids placed on either terminus to create conveniently located restriction sites, or additional amino acids (such as poly His) to aid in purification steps. In addition to recombinant methods, the immunoconjugates, effector moieties, and antibodies of the present disclosure can also be constructed in whole or in part using standard peptide synthesis well known in the art.

Once expressed, the recombinant immunoconjugates, antibodies, and/or effector molecules can be purified according to standard procedures of the art, including ammonium sulfate precipitation, affinity columns, column chromatography, and the like (see, generally, R. Scopes, PROTEIN PURIFICATION, Springer-Verlag, N.Y., 1982). The antibodies, immunoconjugates and effector molecules need not be 100% pure. Once purified, partially or to homogeneity as desired, if to be used therapeutically, the polypeptides should be substantially free of endotoxin.

Methods for expression of antibodies and/or refolding to an appropriate active form, including single chain antibodies, from bacteria such as E. coli have been described and are well-known and are applicable to the antibodies disclosed herein. See, Buchner et al., Anal. Biochem. 205:263-270, 1992; Pluckthun, Biotechnology 9:545, 1991; Huse et al., Science 246:1275, 1989 and Ward et al., Nature 341:544, 1989.

Often, functional heterologous proteins from E. coli or other bacteria are isolated from inclusion bodies and require solubilization using strong denaturants, and subsequent refolding. During the solubilization step, as is well known in the art, a reducing agent must be present to separate disulfide bonds. An exemplary buffer with a reducing agent is: 0.1 M Tris pH 8, 6 M guanidine, 2 mM EDTA, 0.3 M DTE (dithioerythritol). Reoxidation of the disulfide bonds can occur in the presence of low molecular weight thiol reagents in reduced and oxidized form, as described in Saxena et al., Biochemistry 9: 5015-5021, 1970, and especially as described by Buchner et al., supra.

Renaturation is typically accomplished by dilution (for example, 100-fold) of the denatured and reduced protein into refolding buffer. An exemplary buffer is 0.1 M Tris, pH 8.0, 0.5 M L-arginine, 8 mM oxidized glutathione (GSSG), and 2 mM EDTA.

As a modification to the two chain antibody purification protocol, the heavy and light chain regions are separately solubilized and reduced and then combined in the refolding solution. An exemplary yield is obtained when these two proteins are mixed in a molar ratio such that a 5-fold molar excess of one protein over the other is not exceeded. Excess oxidized glutathione or other oxidizing low molecular weight compounds can be added to the refolding solution after the redox-shuffling is completed.

In addition to recombinant methods, the antibodies, labeled antibodies and antigen binding fragments thereof that are disclosed herein can also be constructed in whole or in part using standard peptide synthesis. Solid phase synthesis of the polypeptides of less than about 50 amino acids in length can be accomplished by attaching the C-terminal amino acid of the sequence to an insoluble support followed by sequential addition of the remaining amino acids in the sequence. Techniques for solid phase synthesis are described by Barany & Merrifield, The Peptides: Analysis, Synthesis, Biology. Vol. 2: Special Methods in Peptide Synthesis, Part A. pp. 3-284; Merrifield et al., J. Am. Chem. Soc. 85:2149-2156, 1963, and Stewart et al., Solid Phase Peptide Synthesis, 2nd ed., Pierce Chem. Co., Rockford, Ill., 1984. Proteins of greater length may be synthesized by condensation of the amino and carboxyl termini of shorter fragments. Methods of forming peptide bonds by activation of a carboxyl terminal end (such as by the use of the coupling reagent N, N′-dicylohexylcarbodimide) are well known in the art.

### C. Compositions and Therapeutic Methods

Methods are disclosed herein for the prevention or treatment of an HIV infection, such as an HIV-1 infection. Prevention can include inhibition of infection with HIV-1. The methods include contacting a cell with an effective amount of a human monoclonal antibody disclosed herein that specifically binds gp120, or an antigen binding fragment thereof or a nucleic acid encoding such antibody or antigen binding fragment. The method can also include administering to a subject a therapeutically effective amount of the human monoclonal antibodies to a subject, or an antigen binding fragment thereof or a nucleic acid encoding such antibody or antigen binding fragment, for example the antigen binding fragment can be one or more of the CDRs grafted onto a protein scaffold. In some examples, the antibody, or an antigen binding fragment thereof or a nucleic acid encoding such antibody or antigen binding fragments, can be used in post-exposure prophylaxis. In some examples, an antibody, an antigen binding fragment thereof, or a nucleic acid encoding such antibody or antigen binding fragment, is used to eliminate the viral reservoir. For example a patient on antivirals would be given the antibody, or an antigen binding fragment thereof or a nucleic acid encoding such antibody or antigen binding fragment. In some examples the antibody, or an antigen binding fragment thereof, is modified such that directly cytotoxic to infected cells, or uses natural defenses such as complement, antibody dependent cellular cytotoxicity (ADCC), or phagocytosis by macrophages.

Methods to assay for neutralization activity include, but are not limited to, a single-cycle infection assay as described in Martin et al. (2003) Nature Biotechnology 21:71-76. In this assay, the level of viral activity is measured via a selectable marker whose activity is reflective of the amount of viable virus in the sample, and the IC50 is determined. In other assays, acute infection can be monitored in the PM1 cell line or in primary cells (normal PBMC). In this assay, the level of viral activity can be monitored by determining the p24 concentrations using ELISA. See, for example, Martin et al. (2003) Nature Biotechnology 21:71-76.

HIV infection does not need to be completely eliminated for the composition to be effective. For example, a composition can decrease HIV infection by a desired amount, for example by at least 10%, at least 20%, at least 50%, at least 60%, at least 70%, at least 80%, at least 90%, at least 95%, at least 98%, or even at least 100% (elimination of detectable HIV infected cells), as compared to HIV infection in the absence of the composition. In example, the cell is also contacted with an effective amount of an additional agent, such as anti-viral agent. The cell can be in vivo or in vitro. The methods can include administration of one on more additional agents known in the art. In additional examples, HIV replication can be reduced or inhibited by similar methods. HIV replication does not need to be completely eliminated for the composition to be effective. For example, a composition can decrease HIV replication by a desired amount, for example by at least 10%, at least 20%, at least 50%, at least 60%, at least 70%, at least 80%, at least 90%, at least 95%, at least 98%, or even at least 100% (elimination of detectable HIV), as compared to HIV replication in the absence of the composition. In one example, the cell is also contacted with an effective amount of an additional agent, such as anti-viral agent. The cell can be in vivo or in vitro.

Compositions are provided that include one or more of the antibodies that specifically bind gp120, or an antigen binding fragment thereof or an nucleic acid encoding such antibodies or antigen binding fragments, that are disclosed herein in a carrier. The compositions can be prepared in unit dosage forms for administration to a subject. The amount and timing of administration are at the discretion of the treating physician to achieve the desired purposes. An antibody can be formulated for systemic or local administration. In one example, the antibody that specifically binds gp120, an antigen binding fragment thereof or a nucleic acid encoding such antibody or antigen binding fragment, is formulated for parenteral administration, such as intravenous administration.

The compositions for administration can include a solution of the antibody that specifically binds gp120, or an antigen binding fragment thereof or an nucleic acid encoding such antibodies or antigen binding fragments thereof, dissolved in a pharmaceutically acceptable carrier, such as an aqueous carrier. A variety of aqueous carriers can be used, for example, buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antibody in these formulations can vary widely, and will be selected primarily based on fluid volumes, viscosities, body weight and the like in accordance with the particular mode of administration selected and the subject's needs.

A typical pharmaceutical composition for intravenous administration includes about 0.1 to 10 mg of antibody per subject per day. Dosages from 0.1 up to about 100 mg per subject per day may be used, particularly if the agent is administered to a secluded site and not into the circulatory or lymph system, such as into a body cavity or into a lumen of an organ. Actual methods for preparing administrable compositions will be known or apparent to those skilled in the art and are described in more detail in such publications as Remington's Pharmaceutical Science, 19th ed., Mack Publishing Company, Easton, Pa. (1995).

An antibody, an antigen binding fragment thereof, or a nucleic acid encoding such antibodies or antigen binding fragments thereof, may be provided in lyophilized form and rehydrated with sterile water before administration, although they are also provided in sterile solutions of known concentration. The antibody, an antigen binding fragment thereof or a nucleic acid encoding such antibodies or antigen binding fragments thereof, is then added to an infusion bag containing 0.9% sodium chloride, USP, and typically administered at a dosage of from 0.5 to 15 mg/kg of body weight. Considerable experience is available in the art in the administration of antibody drugs, which have been marketed in the U.S. since the approval of RITUXANO in 1997. An antibody, or an antigen binding fragment thereof, or a nucleic acid encoding such antibodies or antigen binding fragments, can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30 minute period if the previous dose was well tolerated.

A therapeutically effective amount of a human gp120-specific antibody, an antigen binding fragment thereof, or a nucleic acid encoding such antibody or antigen binding fragment thereof, will depend upon the severity of the disease and/or infection and the general state of the patient's health. A therapeutically effective amount of the antibody, antigen binding fragment or nucleic acid is that which provides either subjective relief of a symptom(s) or an objectively identifiable improvement as noted by the clinician or other qualified observer. These compositions can be administered in conjunction with another therapeutic agent, either simultaneously or sequentially.

In one embodiment, administration of the antibody, or antigen binding fragment thereof or nucleic acid encoding such antibody or antigen binding fragment, results in a reduction in the establishment of HIV infection and/or reducing subsequent HIV disease progression in a subject. A reduction in the establishment of HIV infection and/or a reduction in subsequent HIV disease progression encompass any statistically significant reduction in HIV activity. In some embodiments, methods are disclosed for treating a subject with an HIV-1 infection. These methods include administering to the subject a therapeutically effective amount of an antibody, or a nucleic acid encoding the antibody, thereby preventing or treating the HIV-1 infection.

Studies have shown that the rate of HIV transmission from mother to infant is reduced significantly when zidovudine is administered to HIV-infected women during pregnancy and delivery and to the offspring after birth (Connor et al., 1994 Pediatr Infect Dis J 14: 536-541). Several studies of mother-to-infant transmission of HIV have demonstrated a correlation between the maternal virus load at delivery and risk of HIV transmission to the child. The present disclosure provides isolated human monoclonal antibodies, fragments and nucleic acids that are of use in decreasing HIV-transmission from mother to infant. Thus, in some examples, a therapeutically effective amount of a human gp120-specific antibody, an antigen binding fragment thereof or a nucleic acid encoding such antibody or antigen binding fragments thereof, is administered in order to prevent transmission of HIV, or decrease the risk of transmission of HIV, from a mother to an infant. In some examples, a therapeutically effective amount of the antibody, an antigen binding fragment thereof, or a nucleic acid encoding such antibody or antigen binding fragments thereof, is administered to mother and/or to the child at childbirth. In other examples, a therapeutically effective amount of the antibody, antigen binding fragment or nucleic acid is administered to the mother and/or infant prior to breast feeding in order to prevent viral transmission to the infant or decrease the risk of viral transmission to the infant. In some embodiments, both a therapeutically effective amount of the antibody and a therapeutically effective amount of another agent, such as zidovudine, is administered to the mother and/or infant.

For any application, the antibody, antigen binding fragment thereof, or nucleic acid encoding such antibody or antibody binding fragment, can be combined with anti-retroviral therapy. Anti-retroviral drugs are broadly classified by the phase of the retrovirus life-cycle that the drug inhibits. The disclosed antibodies can be administered in conjunction with nucleoside analog reverse-transcriptase inhibitors (such as zidovudine, didanosine, zalcitabine, stavudine, lamivudine, abacavir, emtricitabine, entecavir, and apricitabine), nucleotide reverse transcriptase inhibitors (such as tenofovir and adefovir), non-nucleoside reverse transcriptase inhibitors (such as efavirenz, nevirapine, delavirdine, etravirine, and rilpivirine), protease inhibitors (such as saquinavir, ritonavir, indinavir, nelfinavir, amprenavir, lopinavir, fosamprenavir, atazanavir, tipranavir, and darunavir), entry or fusion inhibitors (such as maraviroc and enfuvirtide), maturation inhibitors, (such as bevirimat and vivecon), or a broad spectrum inhibitors, such as natural antivirals. In some examples, a disclosed antibody or active fragment thereof or nucleic acids encoding such is administered in conjunction with IL-15.

In some examples, a subject is further administered one or more additional antibodies that bind HIV glycoproteins, such as gp120 and gp41. Examples of neutralizing antibodies that can be administered in conjunction with the disclosed antibodies can be found in International Patent Publication No. WO 2011/038290, published Mar. 31, 2011, which is specifically incorporated herein by reference in its entirety.

Single or multiple administrations of the compositions including the antibodies, antigen binding fragments and nucleic acids disclosed herein are administered depending on the dosage and frequency as required and tolerated by the patient. In any event, the composition should provide a sufficient quantity of at least one of the antibodies disclosed herein to effectively treat the patient. The dosage can be administered once, but may be applied periodically until either a therapeutic result is achieved or until side effects warrant discontinuation of therapy. In one example, a dose of the antibody is infused for thirty minutes every other day. In this example, about one to about ten doses can be administered, such as three or six doses can be administered every other day. In a further example, a continuous infusion is administered for about five to about ten days. The subject can be treated at regular intervals, such as monthly, until a desired therapeutic result is achieved. Generally, the dose is sufficient to treat or ameliorate symptoms or signs of disease without producing unacceptable toxicity to the patient.

Controlled-release parenteral formulations can be made as implants, oily injections, or as particulate systems. For a broad overview of protein delivery systems see, Banga, A. J., Therapeutic Peptides and Proteins: Formulation, Processing, and Delivery Systems, Technomic Publishing Company, Inc., Lancaster, Pa., (1995). Particulate systems include microspheres, microparticles, microcapsules, nanocapsules, nanospheres, and nanoparticles. Microcapsules contain the therapeutic protein, such as a cytotoxin or a drug, as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, for example, Kreuter, J., Colloidal Drug Delivery Systems, J. Kreuter, ed., Marcel Dekker, Inc., New York, N.Y., pp. 219-342 (1994); and Tice & Tabibi, Treatise on Controlled Drug Delivery, A. Kydonieus, ed., Marcel Dekker, Inc. New York, N.Y., pp. 315-339, (1992).

Polymers can be used for ion-controlled release of the compositions disclosed herein. Various degradable and nondegradable polymeric matrices for use in controlled drug delivery are known in the art (Langer, Accounts Chem. Res. 26:537-542, 1993). For example, the block copolymer, polaxamer 407, exists as a viscous yet mobile liquid at low temperatures but forms a semisolid gel at body temperature. It has been shown to be an effective vehicle for formulation and sustained delivery of recombinant interleukin-2 and urease (Johnston et al., Pharm. Res. 9:425-434, 1992; and Pec et al., J. Parent. Sci. Tech. 44(2):58-65, 1990). Alternatively, hydroxyapatite has been used as a microcarrier for controlled release of proteins (Ijntema et al., Int. J. Pharm. 112:215-224, 1994). In yet another aspect, liposomes are used for controlled release as well as drug targeting of the lipid-capsulated drug (Betageri et al., Liposome Drug Delivery Systems, Technomic Publishing Co., Inc., Lancaster, Pa. (1993)). Numerous additional systems for controlled delivery of therapeutic proteins are known (see U.S. Pat. No. 5,055,303; U.S. Pat. No. 5,188,837; U.S. Pat. No. 4,235,871; U.S. Pat. No. 4,501,728; U.S. Pat. No. 4,837,028; U.S. Pat. No. 4,957,735; U.S. Pat. No. 5,019,369; U.S. Pat. No. 5,055,303; U.S. Pat. No. 5,514,670; U.S. Pat. No. 5,413,797; U.S. Pat. No. 5,268,164; U.S. Pat. No. 5,004,697; U.S. Pat. No. 4,902,505; U.S. Pat. No. 5,506,206; U.S. Pat. No. 5,271,961; U.S. Pat. No. 5,254,342 and U.S. Pat. No. 5,534,496).

In some examples, a subject is administered the DNA encoding the antibody or antigen binding fragments thereof, for example the antigen binding fragment can be one or more of the CDRs grafted onto a protein scaffold, to provide in vivo antibody production, for example using the cellular machinery of the subject. Immunization by nucleic acid constructs is well known in the art and taught, for example, in U.S. Pat. No. 5,643,578, and U.S. Pat. No. 5,593,972 and U.S. Pat. No. 5,817,637. U.S. Pat. No. 5,880,103 describes several methods of delivery of nucleic acids encoding to an organism. The methods include liposomal delivery of the nucleic acids. Such methods can be applied to the production of an antibody, or antigen binding fragments thereof, by one of ordinary skill in the art.

One approach to administration of nucleic acids is direct administration with plasmid DNA, such as with a mammalian expression plasmid. The nucleotide sequence encoding the disclosed antibody, or antigen binding fragments thereof, can be placed under the control of a promoter to increase expression.

In another approach to using nucleic acids, a disclosed antibody, or antigen binding fragment thereof can also be expressed by attenuated viral hosts or vectors or bacterial vectors. Recombinant vaccinia virus, adeno-associated virus (AAV), herpes virus, retrovirus, cytomegalovirus or other viral vectors can be used to express the antibody. For example, vaccinia vectors and methods useful protocols are described in U.S. Pat. No. 4,722,848. BCG (Bacillus Calmette Guerin) provides another vector for expression of the disclosed antibodies (see Stover, Nature 351:456-460, 1991).

In one embodiment, a nucleic acid encoding a disclosed antibody or antigen binding fragment thereof is introduced directly into cells. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter.

Typically, the DNA is injected into muscle, although it can also be injected directly into other sites. Dosages for injection are usually around 0.5 μg/kg to about 50 mg/kg, and typically are about 0.005 mg/kg to about 5 mg/kg (see, e.g., U.S. Pat. No. 5,589,466).

### D. Diagnostic Methods and Kits

A method is provided herein for the detection of the expression of gp120 in vitro or in vivo. In one example, expression of gp120 is detected in a biological sample, and can be used to detect HIV-1 infection as the presence of HIV-1 in a sample. The sample can be any sample, including, but not limited to, tissue from biopsies, autopsies and pathology specimens. Biological samples also include sections of tissues, for example, frozen sections taken for histological purposes. Biological samples further include body fluids, such as blood, serum, plasma, sputum, spinal fluid or urine.

In several embodiments, a method is provided for detecting AIDS and/or an HIV-1 infection in a subject. The disclosure provides a method for detecting HIV-1 in a biological sample, wherein the method includes contacting a biological sample with the antibody or antigen binding fragment thereof under conditions conducive to the formation of an immune complex, and detecting the immune complex, to detect the gp120 in the biological sample. In one example, the detection of gp120 in the sample indicates that the subject has an HIV infection. In another example, the detection of gp120 in the sample indicates that the subject has AIDS. In another example, detection of gp120 in the sample confirms a diagnosis of AIDS and/or an HIV-1 infection in a subject.

In some embodiments, the disclosed antibodies or antigen binding fragments thereof are used to test vaccines. For example to test if a vaccine composition assumes the same conformation as a gp120 peptide. Thus provided herein is a method for testing a vaccine, wherein the method includes contacting a sample containing the vaccine, such as a gp120 immunogen, with the antibody under conditions conducive to the formation of an immune complex, and detecting the immune complex, to detect the vaccine in the sample. In one example, the detection of the immune complex in the sample indicates that vaccine component, such as such as a gp120 immunogen assumes a conformation capable of binding the antibody or antigen binding fragment.

In one embodiment, the antibody or antigen binding fragment is directly labeled with a detectable label. In another embodiment, the antibody that binds gp120 (the first antibody) is unlabeled and a second antibody or other molecule that can bind the antibody that binds gp120 is utilized. As is well known to one of skill in the art, a second antibody is chosen that is able to specifically bind the specific species and class of the first antibody. For example, if the first antibody is a human IgG, then the secondary antibody may be an anti-human-IgG. Other molecules that can bind to antibodies include, without limitation, Protein A and Protein G, both of which are available commercially.

Suitable labels for the antibody, antigen binding fragment or secondary antibody are described above, and include various enzymes, prosthetic groups, fluorescent materials, luminescent materials, magnetic agents and radioactive materials. Non-limiting examples of suitable enzymes include horseradish peroxidase, alkaline phosphatase, beta-galactosidase, or acetylcholinesterase. Non-limiting examples of suitable prosthetic group complexes include streptavidin/biotin and avidin/biotin. Non-limiting examples of suitable fluorescent materials include umbelliferone, fluorescein, fluorescein isothiocyanate, rhodamine, dichlorotriazinylamine fluorescein, dansyl chloride or phycoerythrin. A non-limiting exemplary luminescent material is luminol; a non-limiting exemplary a magnetic agent is gadolinium, and non-limiting exemplary radioactive labels include ¹²⁵I, ¹³¹I, ³⁵S or ³H.

The immunoassays and methods disclosed herein can be used for a number of purposes. Kits for detecting a polypeptide will typically comprise an antibody that binds gp120, such as any of the antibodies disclosed herein. In some embodiments, an antibody fragment, such as an Fv fragment or a Fab is included in the kit. In a further embodiment, the antibody is labeled (for example, with a fluorescent, radioactive, or an enzymatic label).

In one embodiment, a kit includes instructional materials disclosing means of use. The instructional materials may be written, in an electronic form (such as a computer diskette or compact disk) or may be visual (such as video files). The kits may also include additional components to facilitate the particular application for which the kit is designed. Thus, for example, the kit may additionally contain means of detecting a label (such as enzyme substrates for enzymatic labels, filter sets to detect fluorescent labels, appropriate secondary labels such as a secondary antibody, or the like). The kits may additionally include buffers and other reagents routinely used for the practice of a particular method. Such kits and appropriate contents are well known to those of skill in the art.

In one embodiment, the diagnostic kit comprises an immunoassay. Although the details of the immunoassays may vary with the particular format employed, the method of detecting gp120 in a biological sample generally includes the steps of contacting the biological sample with an antibody which specifically reacts, under immunologically reactive conditions, to gp120. The antibody is allowed to specifically bind under immunologically reactive conditions to form an immune complex, and the presence of the immune complex (bound antibody) is detected directly or indirectly.

### III. EXAMPLES

The following examples are provided to illustrate certain particular features and/or embodiments. These examples should not be construed to limit the disclosure to the particular features or embodiments described.

### Example 1

### Different Gene Composition of Broadly Neutralizing Antibodies to the Conserved CD4-Binding Site of HIV-1 Envelope

Structural studies have defined four conserved regions on the HIV-1 envelope glycoprotein that are vulnerable to neutralizing antibodies and that are the focus of vaccine design efforts. One such region, the initial site of CD4 receptor attachment, is targeted by a class of VRC01-like antibodies that utilize mimicry of the host cell CD4 receptor to achieve potent neutralization. VRC01-like antibody mimicry of CD4 appears to be restricted to two heavy chain variable genes (VH1-2 and VH1-46) that encode the necessary elements within the complementarity determining region-2 (CDRH2) of the antibody heavy chain, suggesting a genetic limitation to eliciting these antibodies during vaccination.

This example illustrates two additional types of antibodies, VRC13 and VRC16 antibodies, that specifically bind to gp120, and compete for CD4 binding to gp120, but, unlike VRC01 and the VRC01-like antibodies, the disclosed antibodies do not adopt a structural configuration that mimics CD4. Neutralization experiments show that VRC13 neutralizes over 80% of circulating HIV-1 isolates at potencies similar to that of antibody VRC01, the prototypic CD4-mimicking antibody. Also, disclosed are the co-crystalized structures of VRC13 and VRC16 with HIV-1 gp120. The structures of these molecules reveal a CDRH3-loop-based mode of gp120 recognition, rather than CDRH2. In addition, and unlike VRC01 antibodies, VRC13 and VRC16 are not derived from a restricted set of VH genes, and V-gene reverted VRC13 and VRC16 bind to gp120 proteins. This finding reveals that effective CD4-binding-site neutralization can be obtained through diverse B cell ontogenies.

### Identification and Characterization of New Antibodies

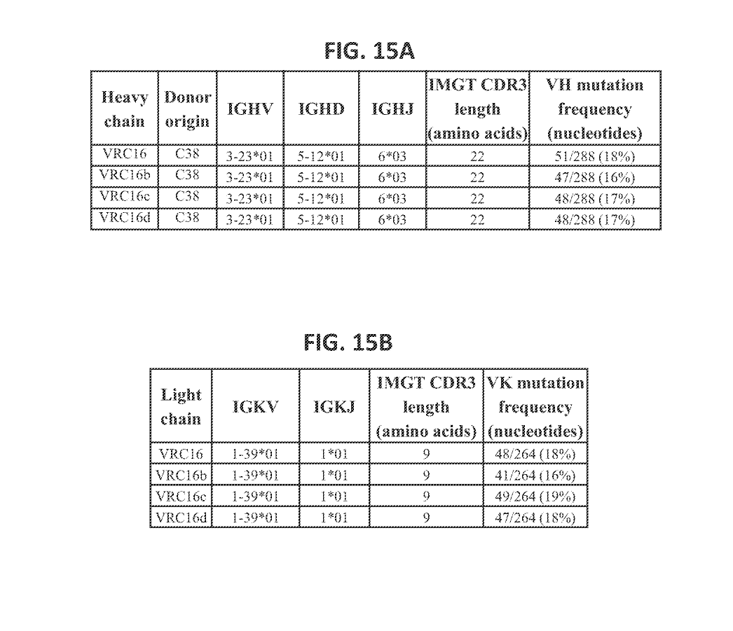

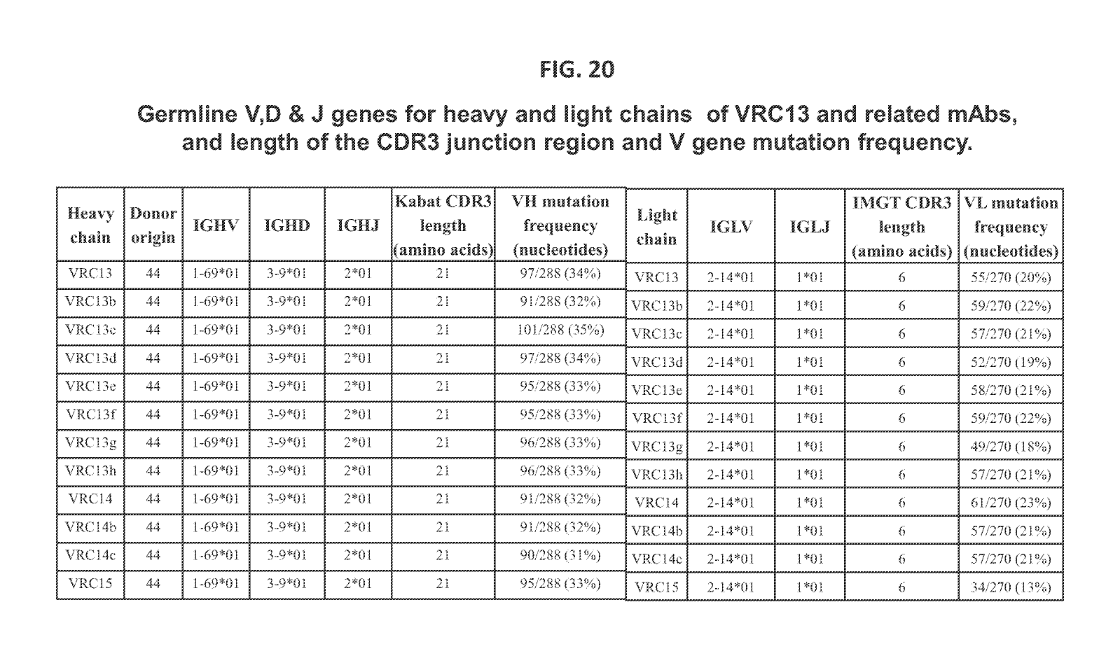

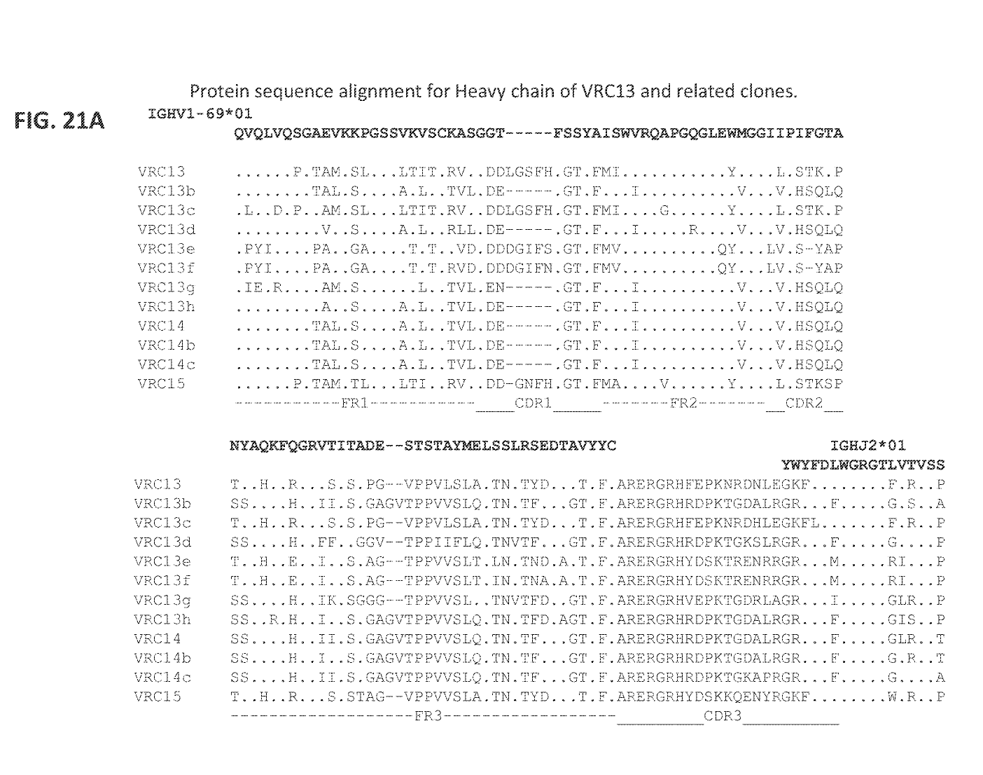

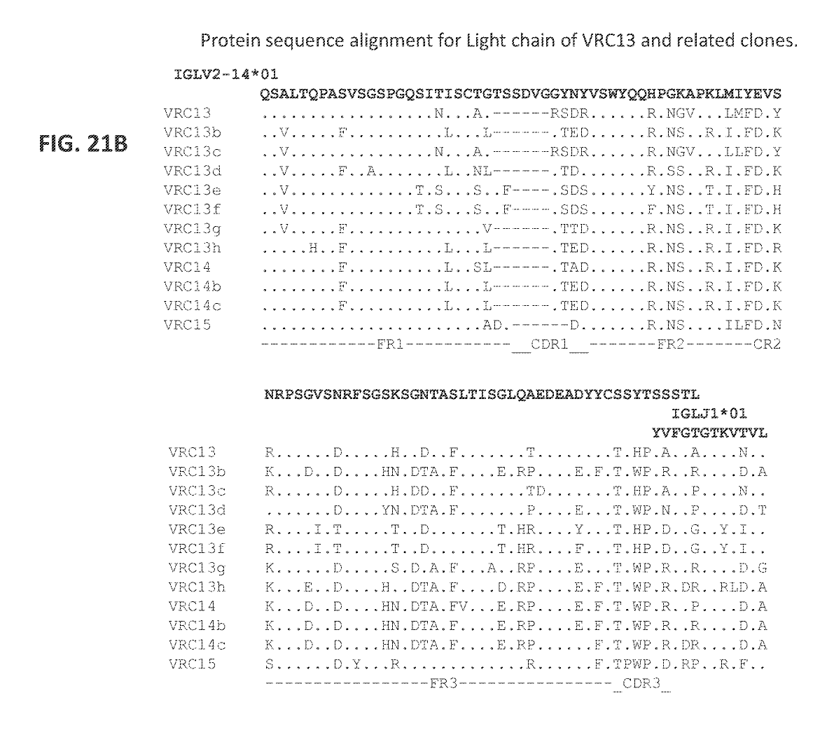

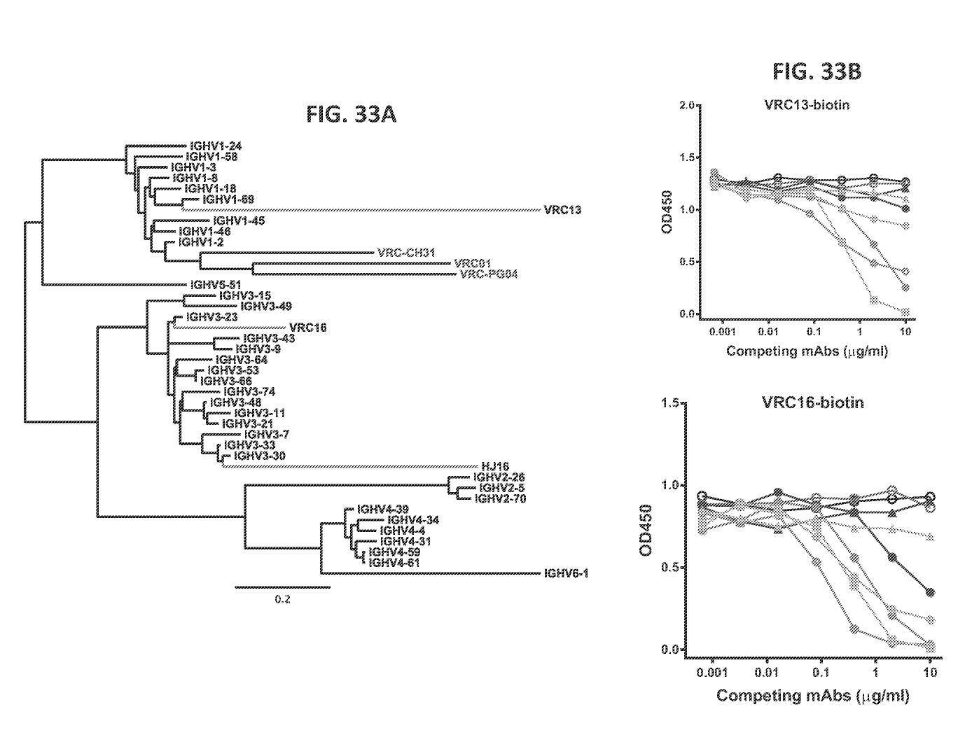

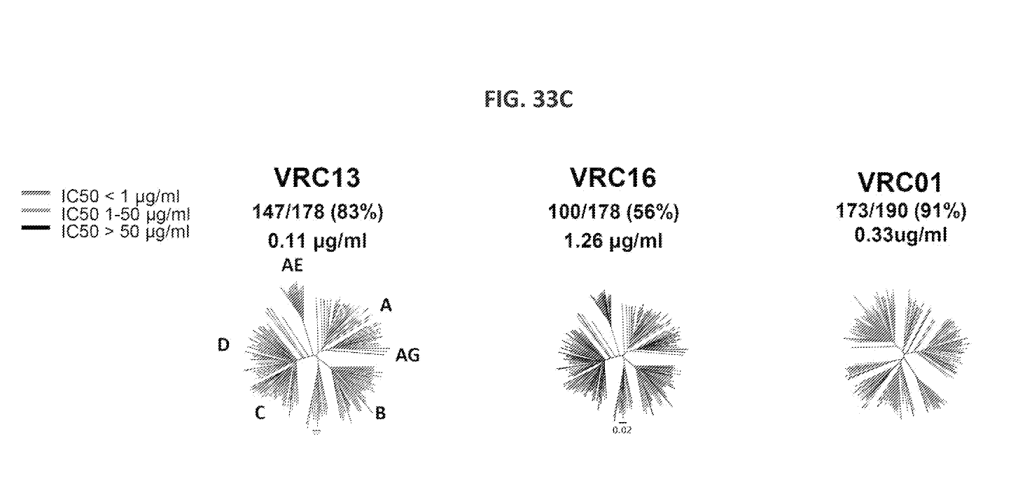

To expand knowledge of broadly neutralizing CD4bs antibody development beyond CD4-mimicry, two donors whose serum demonstrated CD4bs directed neutralizing activity were identified. From these donors, two antibodies, VRC13 and VRC16, that are not VH1-2 restricted, were isolated (FIG. 33A). The gene family derivation for the VRC13 and VRC16 antibodies is shown in FIGS. 15 and 20.

Nucleic acid molecules encoding the VRC13 and VRC16 antibodies were isolated from the B-cells of the HIV-1 infected donors. Genes encoding the heavy and light chain of the VRC13 and VRC16 antibodies were each synthesized and cloned into the CMV/R expression vector containing the constant regions of IgG1. Full-length IgGs were produced by transient transfection using 293fectin (Invitrogen, Carlsbad, Calif.) in 293F cells (Invitrogen) maintained in serum-free free-style medium (Invitrogen). Culture supernatants were harvested 5-6 days after transfection, filtered through a 0.22 μm filter, then followed by IgG1 purification using a recombinant protein-A column (GE Healthcare) for further testing.

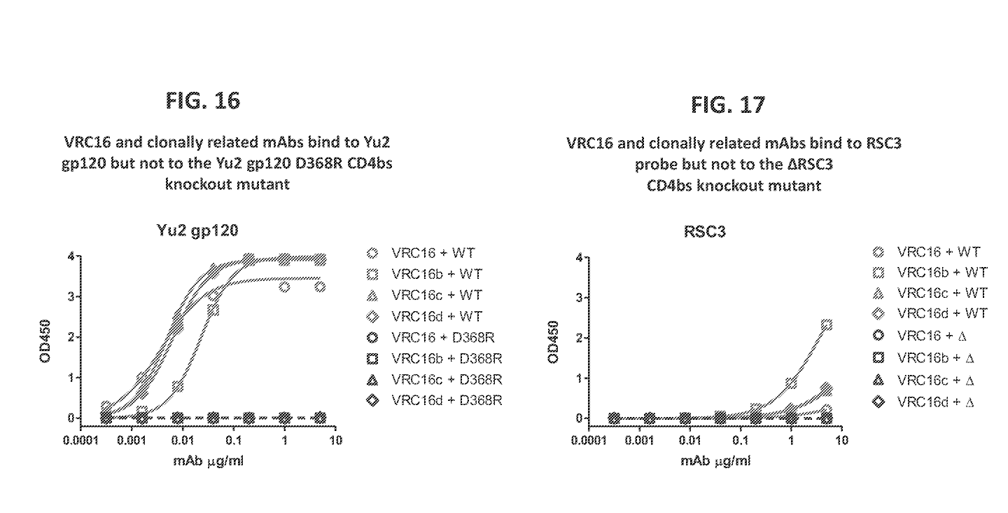

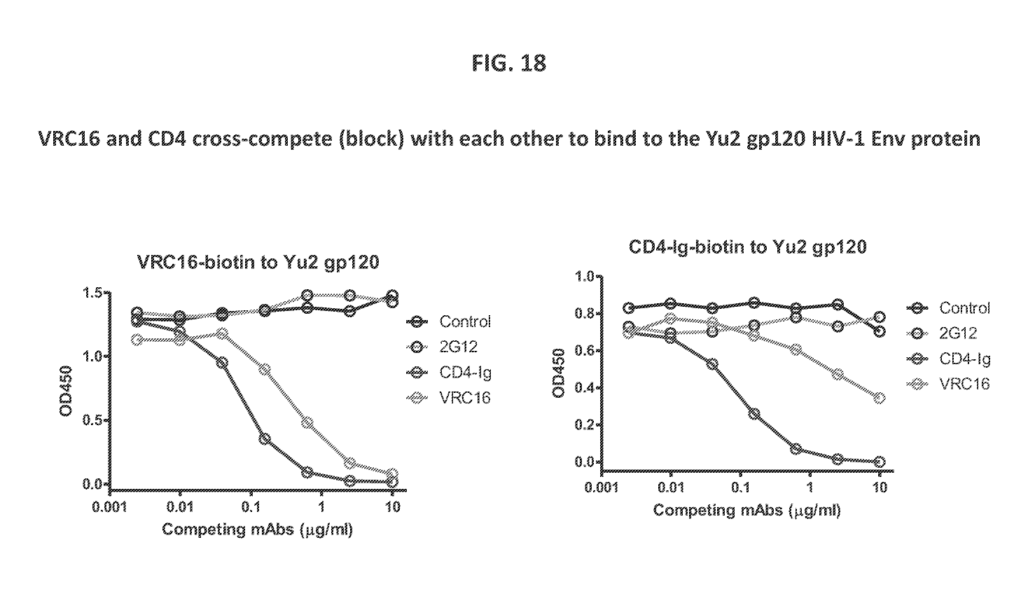

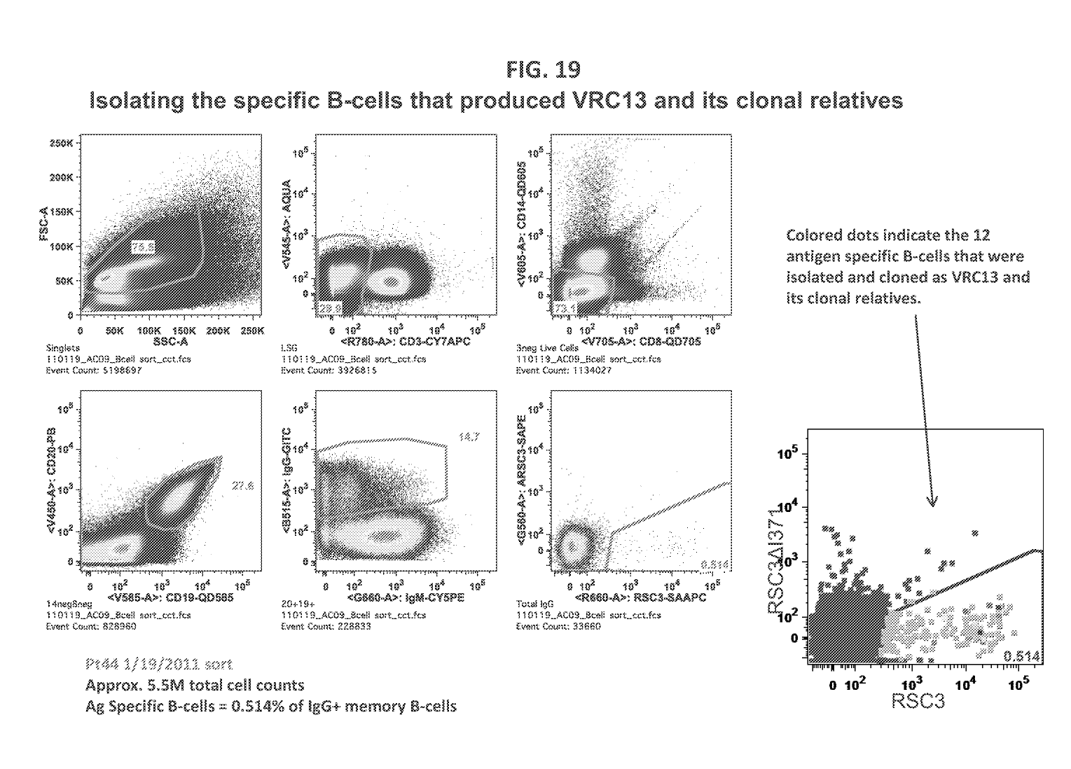

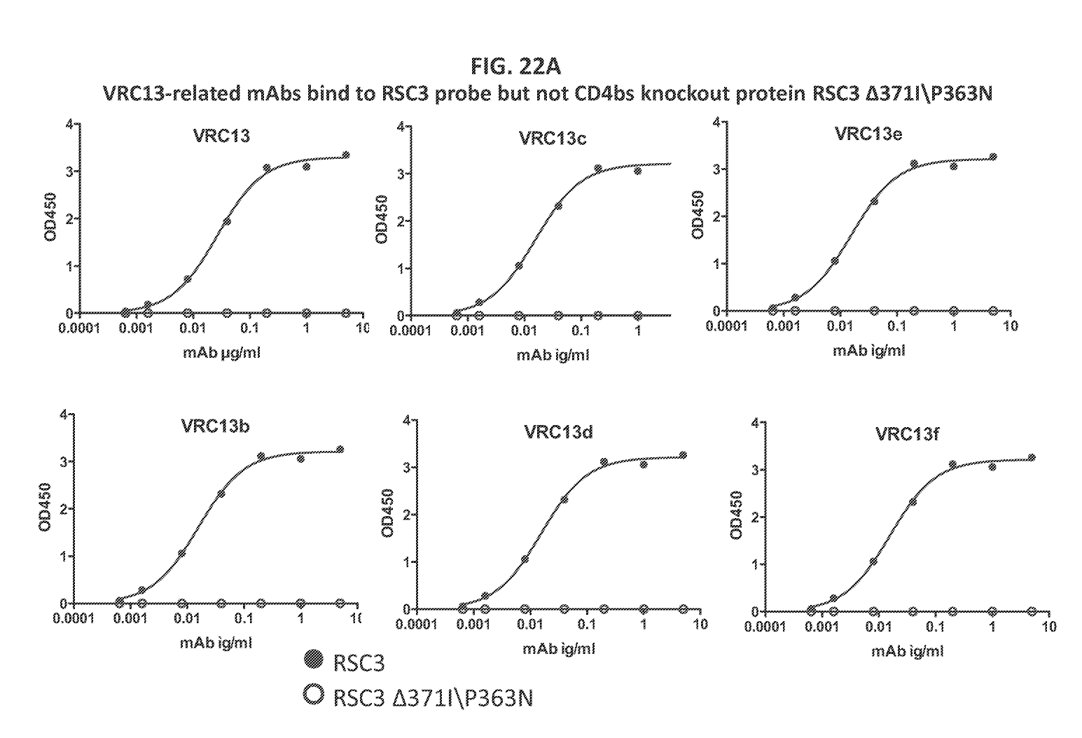

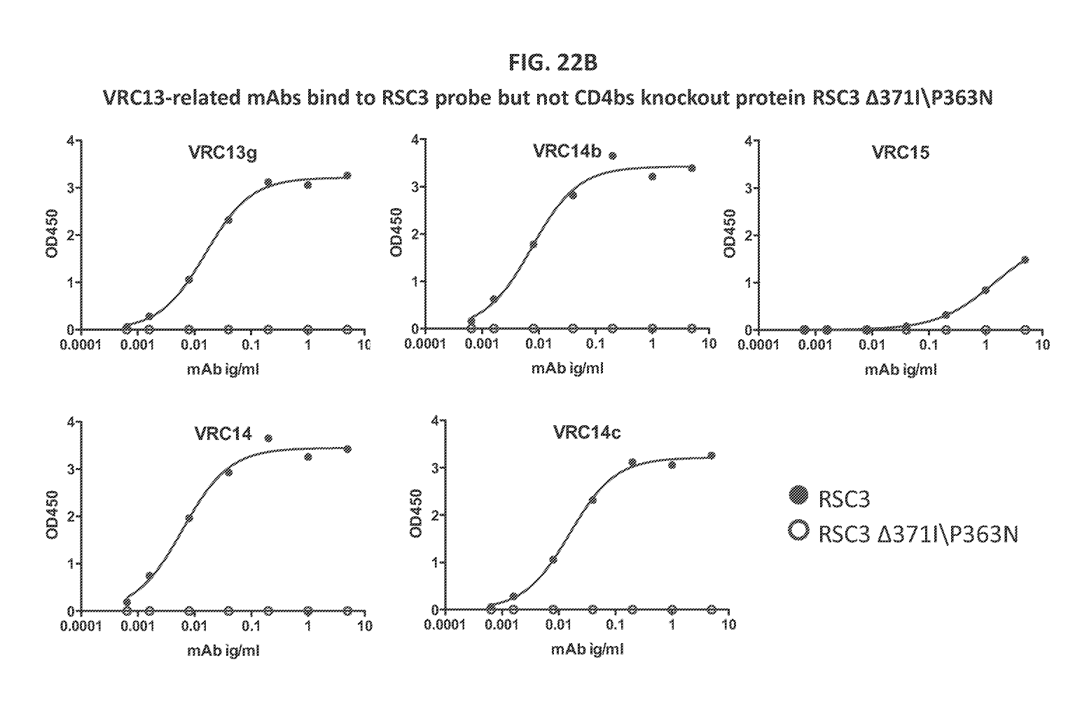

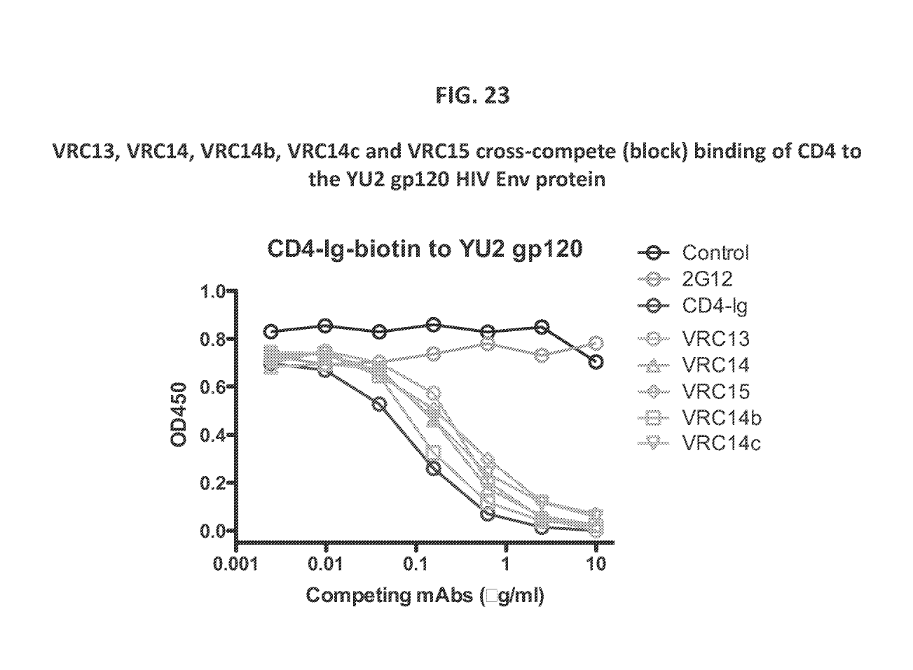

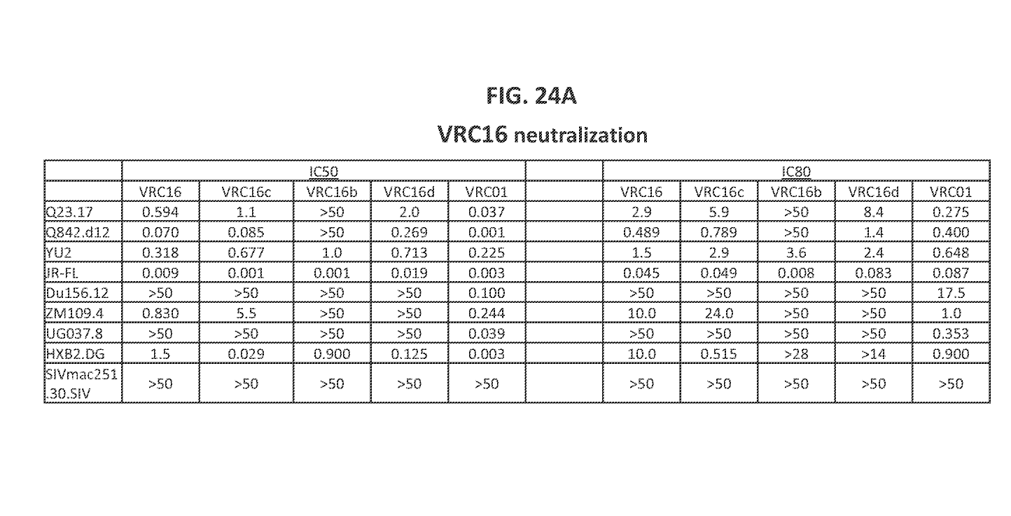

The VRC13 and VRC16 antibodies are directed against the CD4bs, and compete with soluble CD4 for binding to gp120 (FIG. 33B). The VRC13 and VRC16 antibodies bind WT gp120, but do not bind gp120 with a mutated CD4 binding site (FIGS. 16, 17, 22). Further, these antibodies cross-compete for binding to gp120 (see FIGS. 18 and 23). Furthermore, these antibodies neutralize diverse subtypes of HIV-1 with VRC13 displaying comparable breadth and potency to VRC01 (FIGS. 24-25 and 33C).

### Virus Neutralization

Neutralization by the VRC13 and VRC16 monoclonal antibodies was measured using single-round HIV-1 Env-pseudoviruses infection of TZM-b1 cells, substantially as previously described (see, e.g., Wu et al, Science, 329: 856, 2010, incorporated by reference herein in its entirety). HIV-1 Env-pseudoviruses were generated by co-transfection of 293T cells with pSG3AEnv backbone containing a luciferase reporter gene and a second plasmid that expressed HIV-1 Env. At 72 hours post-transfection, supernatants containing pseudovirus were harvested and frozen at −80° C. until further use. In the neutralization assay, 10 μl of 5-fold serially diluted mAb was incubated with 40 μl pseudovirus in a 96-well plate at 37° C. for 30 minutes before addition of TZM-b1 cells. After 2 days of incubation, cells were lysed and the viral infectivity was quantified by measuring luciferase activity with a Victor Light luminometer (Perkin Elmer). The 50% inhibitory concentration (IC50) was calculated as the antibody concentration that reduced infection by 50%. The 80% inhibitory concentration (IC80) was calculated as the antibody concentration that reduced infection by 80%.

IC50 values for neutralization of the indicated HIV-1 strains by VRC16, VRC01, b12 and VRC-PG04 are shown in FIG. 24B-D. IC80 values for neutralization of the indicated HIV-1 strains by VRC13, VRC01, b12 and VRC-PG04 are shown in FIGS. 24E-G. These results are summarized in FIG. 24A. Overall, VRC13 neutralized 56% of gp120 strains tested with an IC50 of less than 50 μg/ml.

IC50 values for neutralization of the indicated HIV-1 strains by VRC13, VRC01, b12 and VRC-PG04 are shown in FIG. 25B-D. IC80 values for neutralization of the indicated HIV-1 strains by VRC13, VRC01, b12 and VRC-PG04 are shown in FIGS. 25E-G. These results are summarized in FIG. 24A. Overall, VRC13 neutralized 83% of gp120 strains tested with an IC50 of less than 50 μg/ml.

### Structures of VRC13 and VRC16

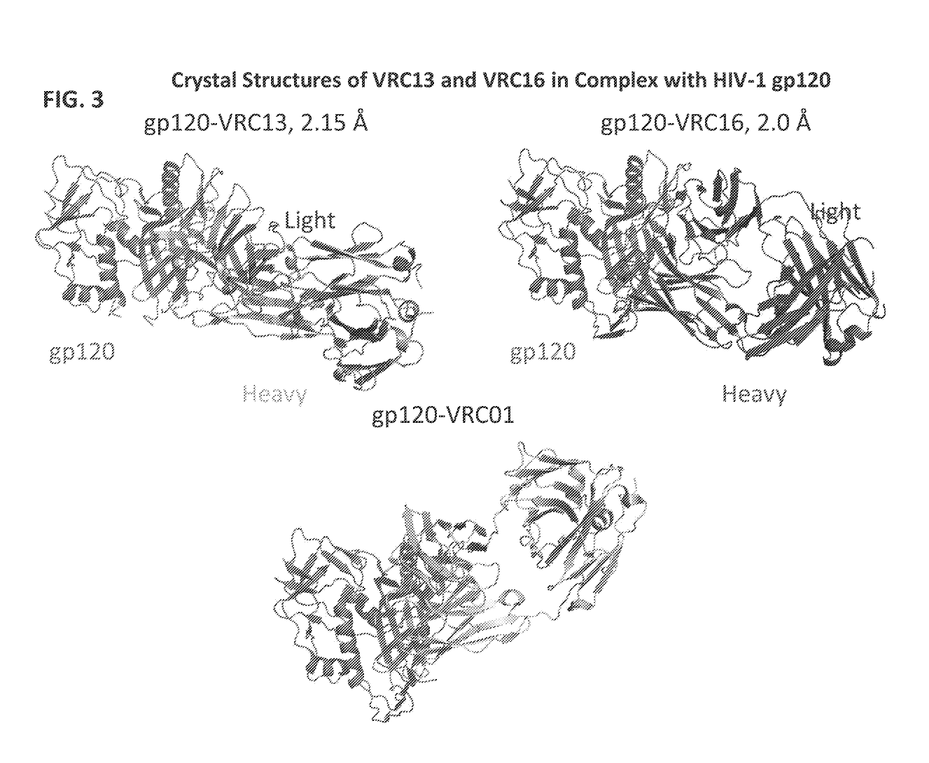

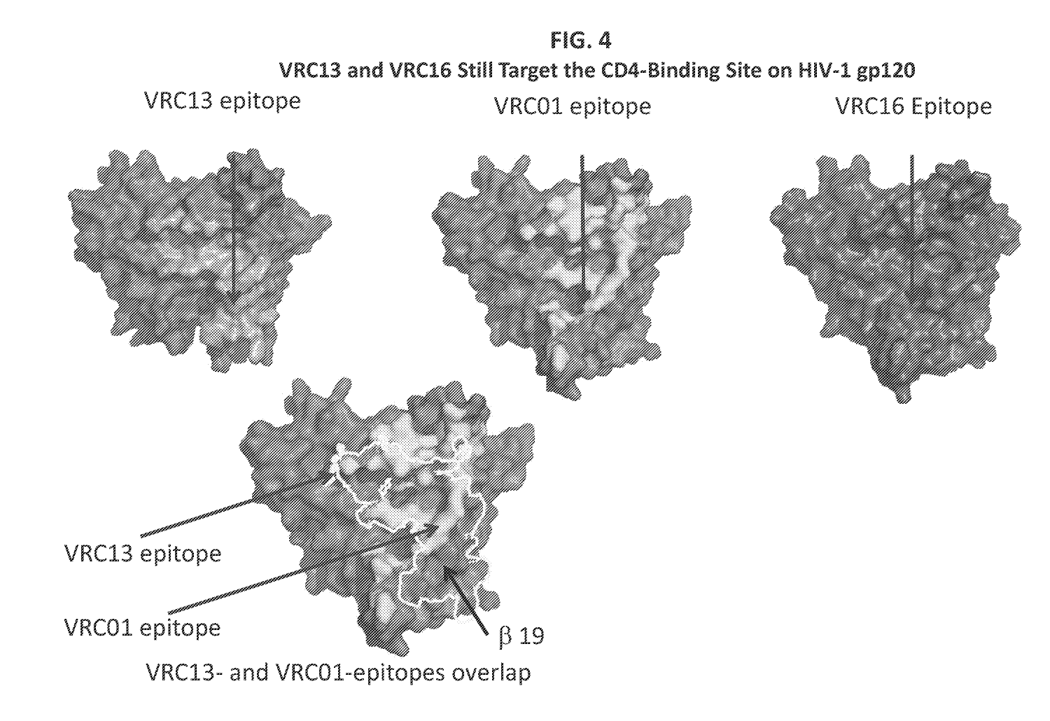

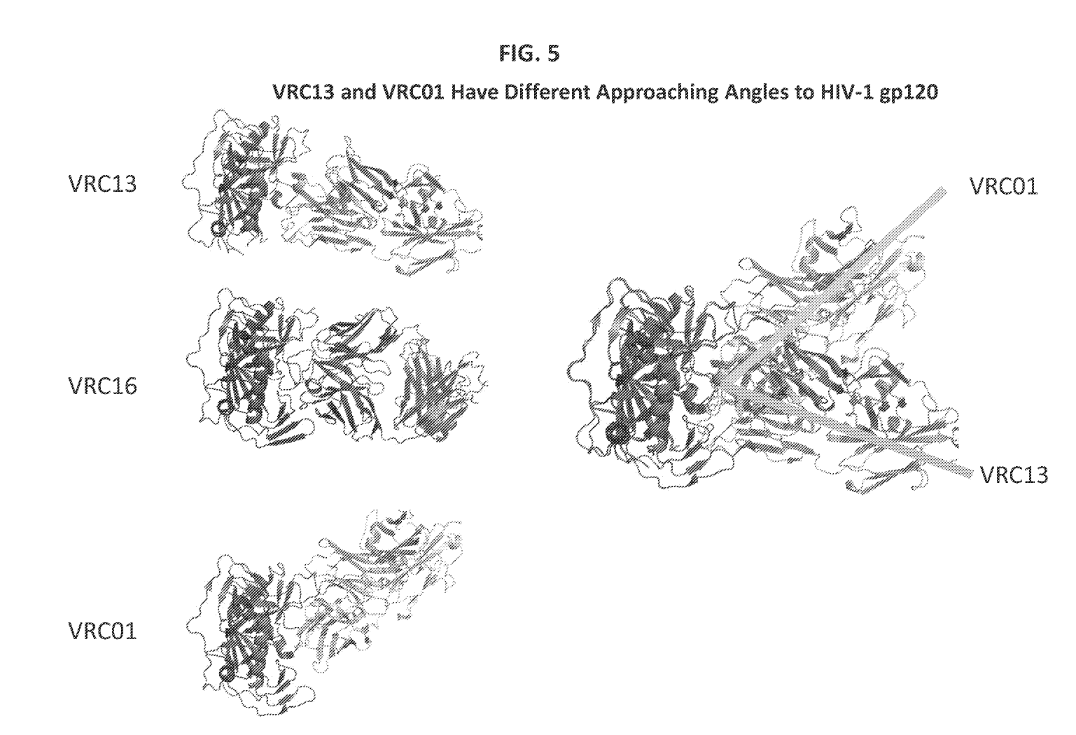

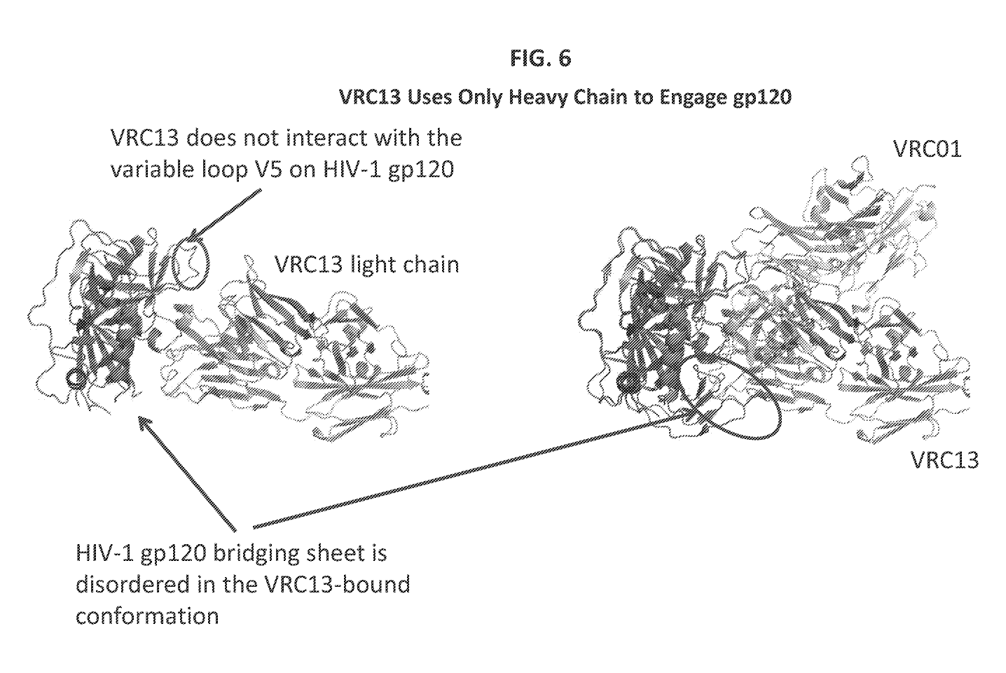

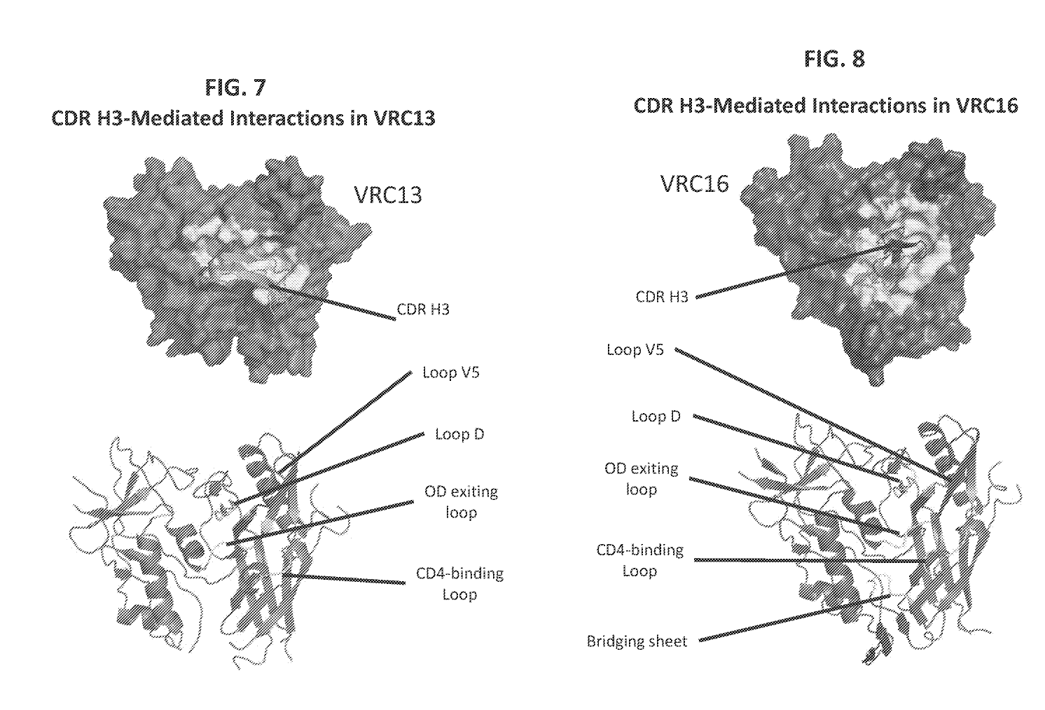

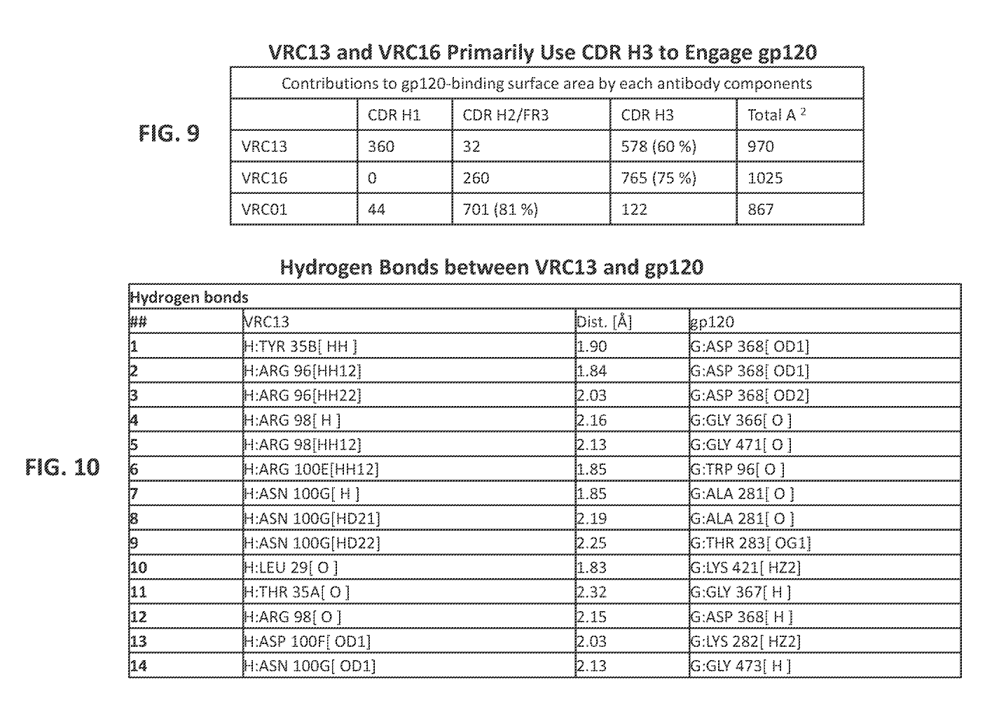

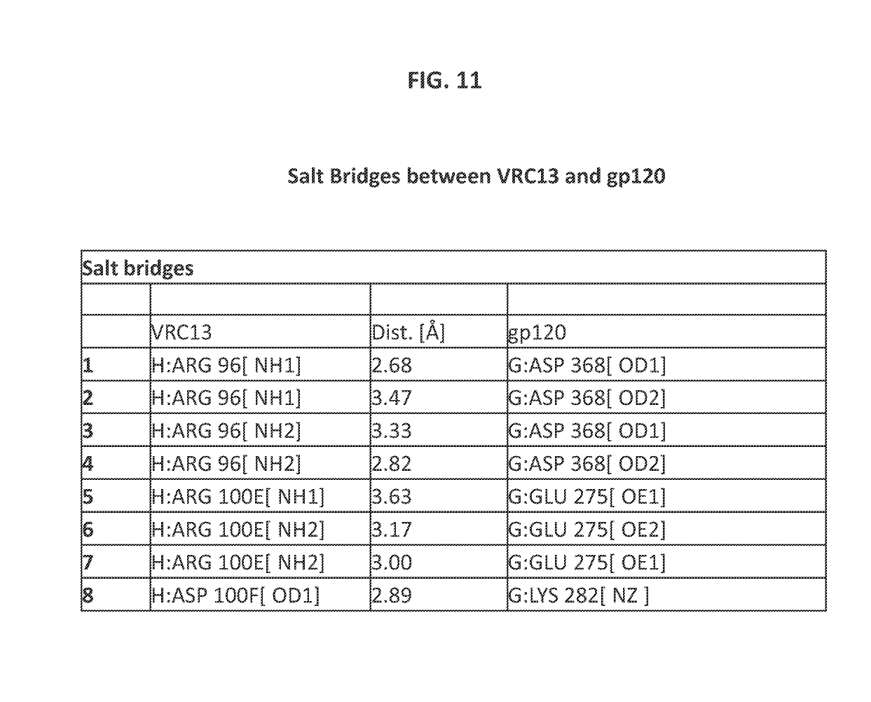

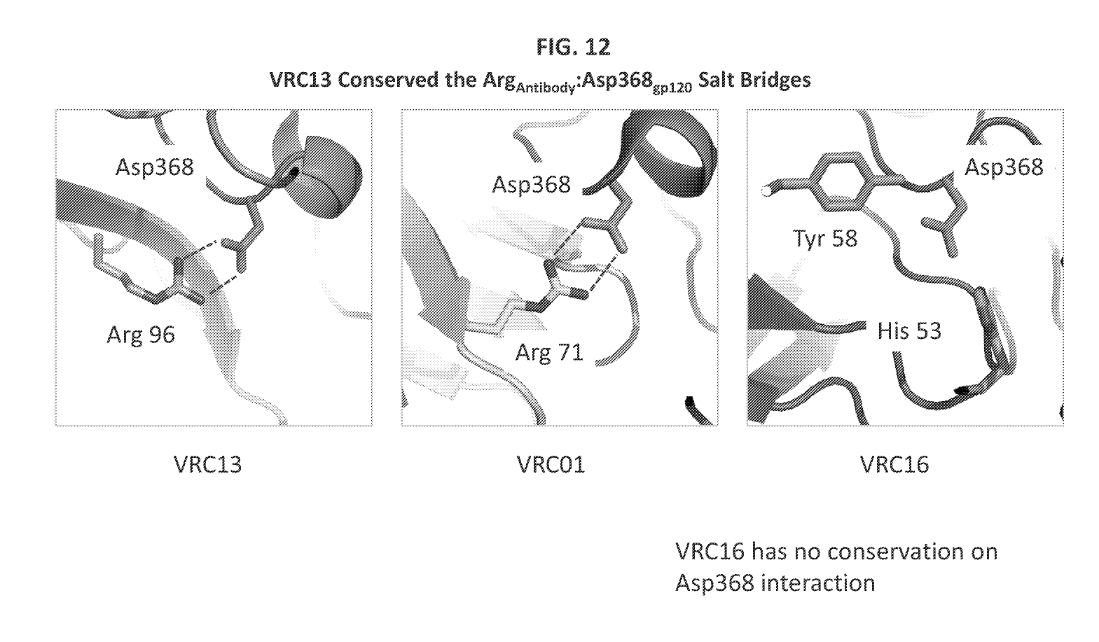

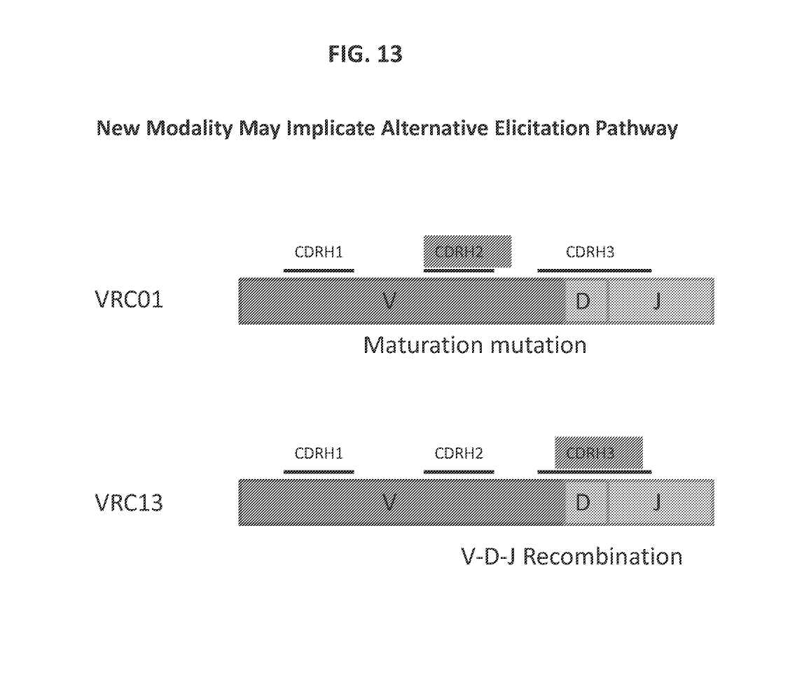

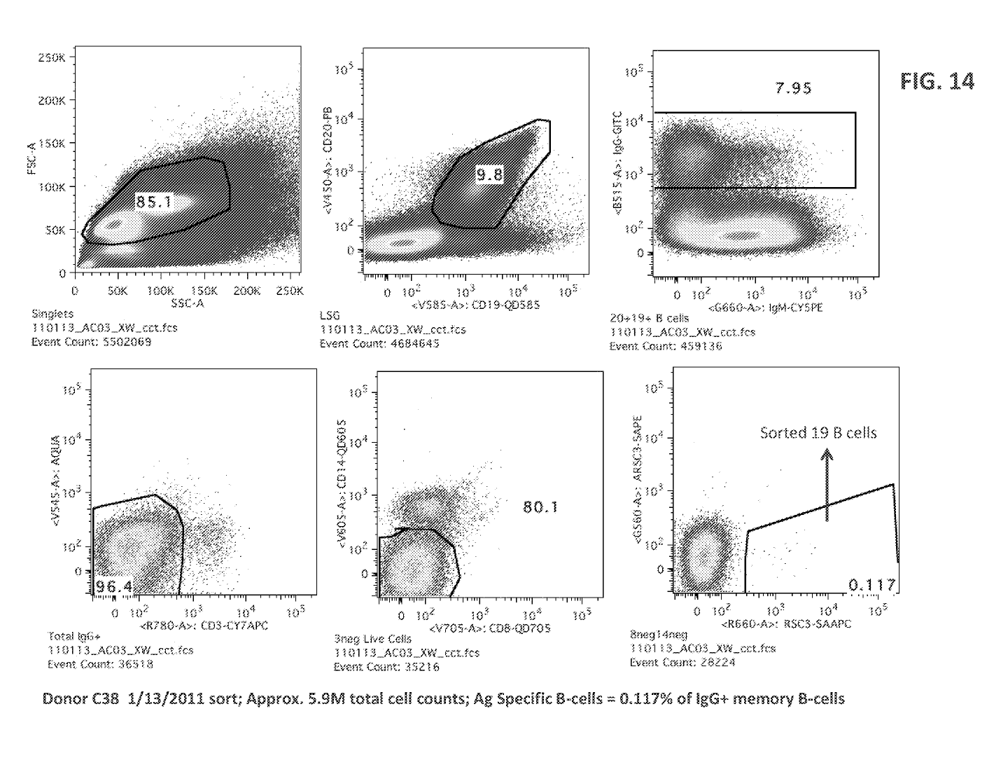

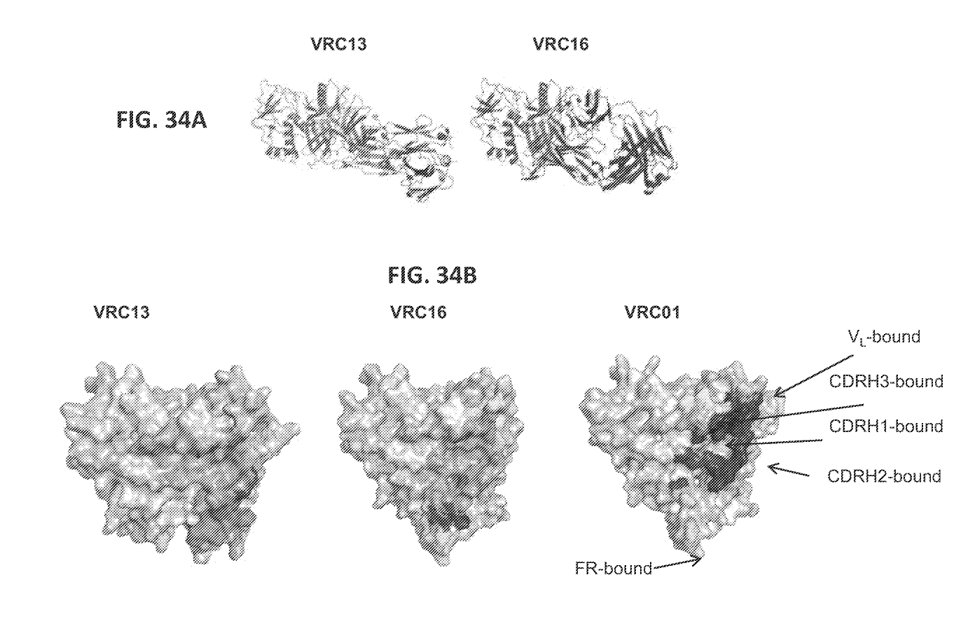

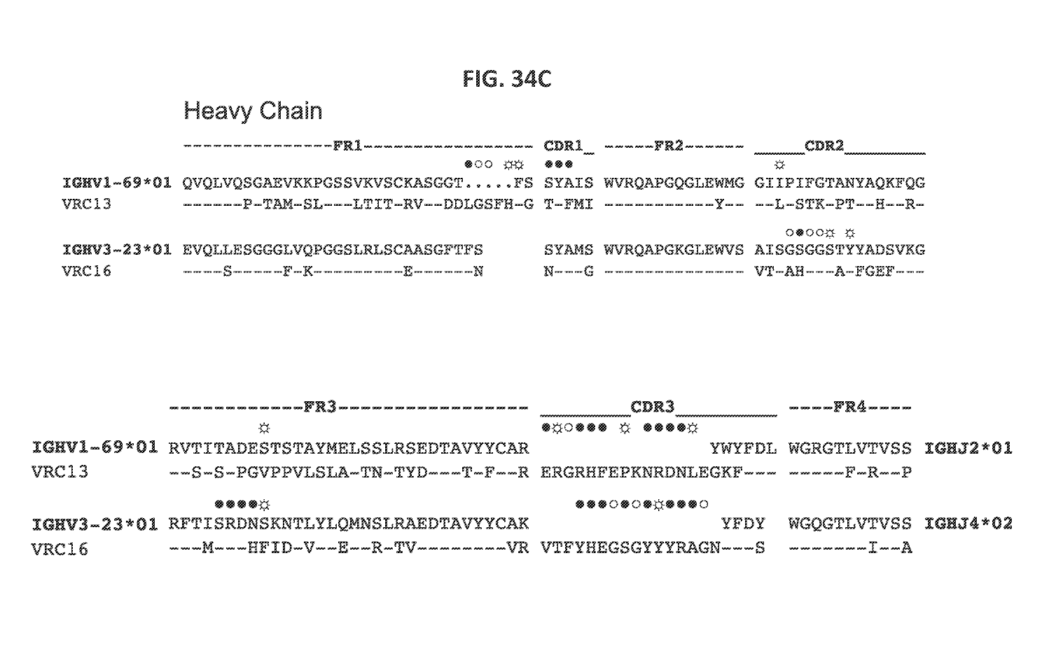

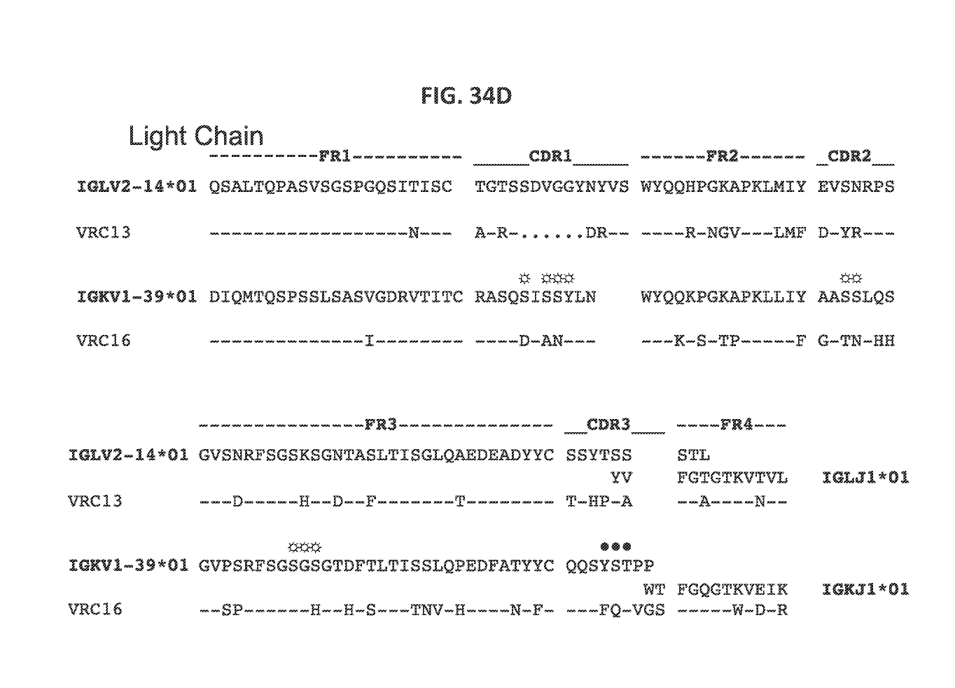

To investigate how the VRC13 and VRC16 antibodies interact with gp120, the structures of VRC13 and VRC16 were solved with atomic level detail (see, e.g., FIGS. 3-8, 12, 34A). The epitope of the VRC13 and VRC16 antibodies is similar to VRC01 and overlaps the CD4bs; however, the VRC13 and VRC16 antibodies mainly interact with gp120 with CDRH3, while VRC01 mainly interacts with CDRH2 (FIG. 34B). The VRC13 and VRC16 antibodies have undergone affinity maturation and are highly mutated when compared at the protein level of their respective germline V and J genes (FIG. 34B). Although both VRC13 and VRC16 utilize CDRH3 to contact gp120, VRC16 also has CDRL3 contact sites, while VRC13 is heavy-chain only binding antibody (FIG. 34B). Furthermore, the nature of these contact-sites and the specific residues involved in the antibody paratope are indicated in FIG. 34C.

The structure of VRC13 indicates a mode of recognition rotated by 45 degrees and translated ˜10 Å from that of VRC01, although both VRC01 and VRC13 utilize similar angles of approach. Unlike VRC01-like antibodies, which feature gp120 contacts primarily in the heavy chain 2^{nd} complementarity determining region (CDR H2), VRC13 utilizes a long heavy chain CDR H3 to contact the CD4-binding site. Overall, the structural details of VRC13 do not mimic those of CD4.

Broad and potent neutralization at the CD4-binding site is not limited to the VRC01-mode of CD4 mimicry. A new mode of effective HIV-1 neutralization, which is defined by the VRC13-gp120 structure and utilizes CDR H3 recognition, can serve as an additional template for the design of an effective HIV-1 vaccine. The natural diversity of the CDR H3—a product of V-D-J recombination can provide advantages in the elicitation of VRC13 antibodies.

The resolved crystal structure of VRC13 and VRC16, bound to gp120, demonstrates that these antibodies have a different angle of approach and recognize a different set of contacts on gp120, compared to VRC01 (see, e.g., FIGS. 3-7). Thus, although VRC01, VRC13, VRC16 all compete with CD4 for binding to gp120, and each of these antibodies specifically binds to a portion of the CD4 binding site on gp120, VRC13 and VRC16 have different genetic derivation than VRC01 and bind via a different structural mode of recognition. The data demonstrate that the CD4bs of HIV-1 envelope contains multiple targets of broadly neutralizing antibodies that arise from different founder B cells with different IGHV gene composition.

Based on the crystal structure of the VRC13 antibody with gp120, the contacts that this antibody makes with gp120 were determined. The gp120 residues that interact with VRC13 heavy chain are listed in FIGS. 31A-31C. The VRC13 heavy chain contacts with gp120 are listed in FIG. 31D-31F. VRC13 light chain does not interact with gp120.

Based on the crystal structure of the VRC16 antibody with gp120, the contacts that this antibody makes with gp120 were determined. The VRC16 heavy chain contacts with gp120 are listed in FIG. 32A. The gp120 residues that interact with VRC16 heavy chain are listed in FIG. 32B. The VRC16 light chain contacts with gp120 are listed in FIG. 32C. The gp120 residues that interact with VRC16 light chain are listed in FIG. 32D.

### V-Gene Reversion Analysis

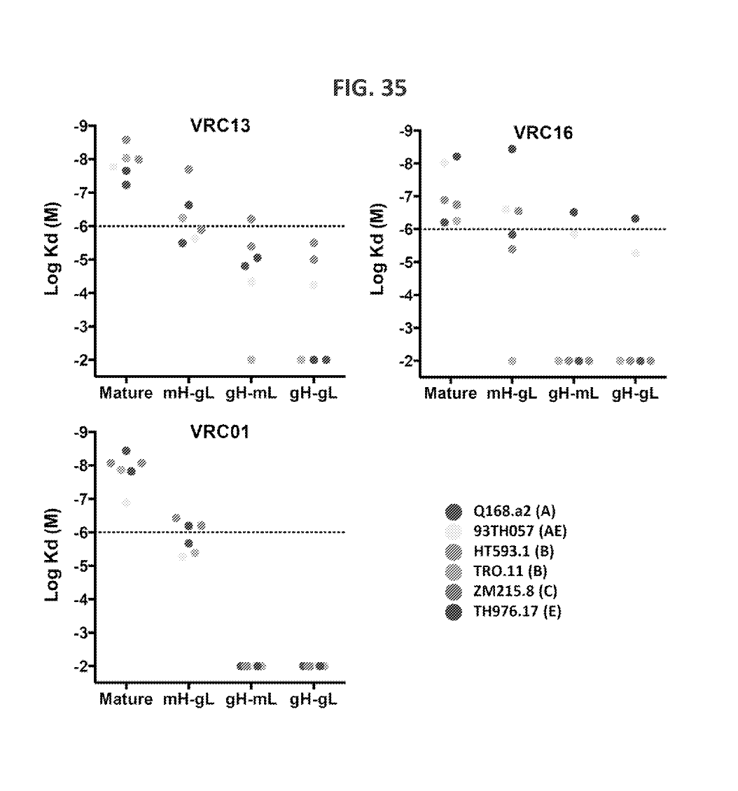

To investigate the role of affinity maturation in CDR-loop based gp120 recognition, the V-gene of the VRC13 and VRC16 antibodies was reverted to germline sequence. The sequences of the reverted VRC13 heavy chain (VRC13gH; SEQ ID NO: 77), reverted VRC13 light chain (VRC13gL; SEQ ID NO: 78), reverted VRC16 heavy chain (VRC16gH; SEQ ID NO: 79), reverted VRC16 light chain (VRC16gL; SEQ ID NO: 80) are provided herein. Mature VRC13, VRC16, and VRC01, V-gene reverted (gH-gL) as well as chimeric versions (mH-gL, gH-mL) were all tested for binding using surface plasma resonance to diverse gp120s (FIG. 35). VRC01 bound to gp120s only when the affinity matured VH gene was present; however, VRC13 and VRC16 specifically bound to multiple gp120s even in the absence of somatic mutations in their heavy and light chain V-genes. These data indicate that the CDRH3 and CDRL3 of these newly isolated antibodies VRC13 and VRC16 are sufficient for binding to HIV Env protein as opposed to VRC01, which requires the mature heavy chain.

### Example 2

### Antibodies Specific to Gp120 for Detecting HIV-1 in a Sample or a Subject

This example describes the use of HIV-1 monoclonal neutralizing antibodies specific to gp120 for the detection of HIV-1 in a sample or a subject. This example further describes the use of these antibodies to confirm the diagnosis of HIV-1 in a subject.

A biological sample, such as a blood sample, is obtained from the patient diagnosed with, undergoing screening for, or suspected of having an HIV-1 infection. A blood sample taken from a patient who is not infected is used as a control, although a standard result can also be used as a control. An ELISA is performed to detect the presence of HIV-1 in the blood sample. Proteins present in the blood samples (the patient sample and control sample) are immobilized on a solid support, such as a 96-well plate, according to methods well known in the art (see, for example, Robinson et al., Lancet 362:1612-1616, 2003, incorporated herein by reference). Following immobilization, HIV-1 monoclonal neutralizing antibodies specific to gp120 that are directly labeled with a fluorescent marker are applied to the protein-immobilized plate. The plate is washed in an appropriate buffer, such as PBS, to remove any unbound antibody and to minimize non-specific binding of antibody. Fluorescence can be detected using a fluorometric plate reader according to standard methods. An increase in fluorescence intensity of the patient sample, relative to the control sample, indicates the gp120 antibody specifically bound proteins from the blood sample, thus detecting the presence of HIV-1 protein in the sample. Detection of HIV-1 protein in the patient sample indicates the patient has HIV-1, or confirms diagnosis of HIV-1 in the subject.

### Example 3

### HIV-1 Monoclonal Neutralizing Antibodies Specific for Gp120 for the Treatment of HIV-1

This example describes a particular method that can be used to treat HIV in a human subject by administration of one or more gp120-specific human neutralizing mAbs. Although particular methods, dosages, and modes of administrations are provided, one skilled in the art will appreciate that variations can be made without substantially affecting the treatment.

Based upon the teaching disclosed herein, HIV-1 can be treated by administering a therapeutically effective amount of one or more of the neutralizing mAbs described herein, thereby reducing or eliminating HIV infection.

### Screening Subjects

In particular examples, the subject is first screened to determine if they have an HIV infection. Examples of methods that can be used to screen for HIV infection include a combination of measuring a subject's CD4+ T cell count and the level of HIV in serum blood levels. Additional methods using the gp120-specific mAbs described herein can also be used to screen for HIV.

In some examples, HIV testing consists of initial screening with an enzyme-linked immunosorbent assay (ELISA) to detect antibodies to HIV, such as to HIV-1. Specimens with a nonreactive result from the initial ELISA are considered HIV-negative unless new exposure to an infected partner or partner of unknown HIV status has occurred. Specimens with a reactive ELISA result are retested in duplicate. If the result of either duplicate test is reactive, the specimen is reported as repeatedly reactive and undergoes confirmatory testing with a more specific supplemental test (e.g., Western blot or an immunofluorescence assay (IFA)). Specimens that are repeatedly reactive by ELISA and positive by IFA or reactive by Western blot are considered HIV-positive and indicative of HIV infection. Specimens that are repeatedly ELISA-reactive occasionally provide an indeterminate Western blot result, which may be either an incomplete antibody response to HIV in an infected person, or nonspecific reactions in an uninfected person. IFA can be used to confirm infection in these ambiguous cases. In some instances, a second specimen will be collected more than a month later and retested for subjects with indeterminate Western blot results. In additional examples, nucleic acid testing (e.g., viral RNA or proviral DNA amplification method) can also help diagnosis in certain situations.

The detection of HIV in a subject's blood is indicative that the subject is infected with HIV and is a candidate for receiving the therapeutic compositions disclosed herein. Moreover, detection of a CD4+ T cell count below 350 per microliter, such as 200 cells per microliter, is also indicative that the subject is likely to have an HIV infection.

Pre-screening is not required prior to administration of the therapeutic compositions disclosed herein

### Pre-Treatment of Subjects

In particular examples, the subject is treated prior to administration of a therapeutic agent that includes one or more antiretroviral therapies known to those of skill in the art. However, such pre-treatment is not always required, and can be determined by a skilled clinician.

### Administration of Therapeutic Compositions

Following subject selection, a therapeutically effective dose of a gp120-specific neutralizing mAb described herein is administered to the subject (such as an adult human or a newborn infant either at risk for contracting HIV or known to be infected with HIV). Additional agents, such as anti-viral agents, can also be administered to the subject simultaneously or prior to or following administration of the disclosed agents. Administration can be achieved by any method known in the art, such as oral administration, inhalation, intravenous, intramuscular, intraperitoneal, or subcutaneous.

The amount of the composition administered to prevent, reduce, inhibit, and/or treat HIV or a condition associated with it depends on the subject being treated, the severity of the disorder, and the manner of administration of the therapeutic composition. Ideally, a therapeutically effective amount of an agent is the amount sufficient to prevent, reduce, and/or inhibit, and/or treat the condition (e.g., HIV) in a subject without causing a substantial cytotoxic effect in the subject. An effective amount can be readily determined by one skilled in the art, for example using routine trials establishing dose response curves. As such, these compositions may be formulated with an inert diluent or with an pharmaceutically acceptable carrier.

In one specific example, antibodies are administered at 5 mg per kg every two weeks or 10 mg per kg every two weeks depending upon the particular stage of HIV. In an example, the antibodies are administered continuously. In another example, antibodies or antibody fragments are administered at 50 μg per kg given twice a week for 2 to 3 weeks.

Administration of the therapeutic compositions can be taken long term (for example over a period of months or years).

### Assessment

Following the administration of one or more therapies, subjects with HIV can be monitored for reductions in HIV levels, increases in a subject's CD4+T cell count, or reductions in one or more clinical symptoms associated with HIV. In particular examples, subjects are analyzed one or more times, starting 7 days following treatment. Subjects can be monitored using any method known in the art. For example, biological samples from the subject, including blood, can be obtained and alterations in HIV or CD4+T cell levels evaluated.

### Additional Treatments

In particular examples, if subjects are stable or have a minor, mixed or partial response to treatment, they can be re-treated after re-evaluation with the same schedule and preparation of agents that they previously received for the desired amount of time, including the duration of a subject's lifetime. A partial response is a reduction, such as at least a 10%, at least 20%, at least 30%, at least 40%, at least 50%, or at least 70% in HIV infection, HIV replication or combination thereof. A partial response may also be an increase in CD4+ T cell count such as at least 350 T cells per microliter.

In view of the many possible embodiments to which the principles of our invention may be applied, it should be recognized that illustrated embodiments are only examples of the invention and should not be considered a limitation on the scope of the invention. Rather, the scope of the invention is defined by the following claims. We therefore claim as our invention all that comes within the scope and spirit of these claims.

1. An isolated monoclonal antibody, or antigen binding fragment thereof, comprising a heavy chain variable region, wherein the heavy chain variable region comprises a heavy chain complementarity determining region (HCDR) 1, a HCDR2, and a HCDR3, comprising amino acids
(A) 36-40, 55-71, and 104-124 of SEQ. ID NO: 5 (VRC13 VH), respectively;
(B) 31-35, 50-66, and 101-121 of SEQ ID NO: 6 (VRC13b VH), respectively;
(C) 36-40, 55-71, and 104-124 of SEQ ID NO: 7 (VRC13c VH), respectively;
(D) 31-35, 50-66 and 99-119 of SEQ ID NO: 8 (VRC13d VH), respectively;
(E) 36-40, 55-71, and 103-123 of SE ID NO: 9 (VRC13e VH) respectively;
(F) 36-40, 55-71, and 103˜123 of SEQ. ID NO 10 (VRC13f VH) respectively;
(G) 31-66, and 99-119 of SEQ NO: 11 (VRC13g VH) respectively;
(H) 31-35, 50-66, and 101-121 of SEQ ID NO: 12 (VRC13h VH) respectively;
(I) 31-35, 50-66, and 101-121 of SEQ ID NO: 13 (VRC14 VH) respectively;
(J) 31-35, 50-66, and 101-121 of SEQ ID NO: 14 (VRC14b VH), respectively;
(K) 31-35, 50-66, and 101-121 of SEQ ID NO: 15 (VRC14c VH), respectively;
(L) 35-39, 54-70, and 103-123 of SEQ ID NO: 16 (VRC15 VH), respectively;
(M) 31-35, 50-66, and 99-118, of SEQ ID NO: 29 (VRC16), respectively;
(N) 31-35, 50-66, and 99-118 of SEQ m NO: 30 (VRC16b), respectively;
(O) 31-35, 50-66, and 99-118 of SEQ ID NO: 31 (VRC16c), respectively; or
(P) 31-35, 50-66, and 99-118 of SEQ ID NO: 32 (VRC 16d), respectively; and
wherein the antibody or antigen binding fragment specifically binds to gp120.

2. The antibody or antigen binding fragment of claim 1, wherein the antibody or antigen binding fragment neutralizes HIV-1 infection.

3. The antibody or antigen binding fragment of claim 1, further comprising a light chain variable region.

4-8. (canceled)

9. The antibody or antigen binding fragment of claim 3, wherein the light chain variable region comprises a light chain complementarity determining region (LCDR1), a LCDR2, and a LCDR3, wherein the antibody or antigen binding fragment comprises the HCDR1, HCDR2 and HCDR3 according to
(A) and the LCDR1, LCDR2, and LCDR3 comprise amino acids 23-30, 46-52, and 85-90, of SEQ ID NO: 17 (VRC13 VL), respectively;
(B) and the LCDR1, LCDR2, and LCDR3 comprise amino acids 23-30, 46-52, and 85-90 of SEQ ID NO: 18 (VRC13b VL), respectively;
(C) and the LCDR1, LCDR2, and LCDR3 comprise amino acids 23-30, 46-52, and 85-90 of SEQ ID NO: 19 (VRC13c VL), respectively
(D) and the LCDR1, LCDR2, and LCDR3 comprise amino acids 23-30, 46-52 and 85-90 of SEQ ID NO: 20 (VRC13d VL), respectively;
(E) and the LCDR1, LCDR2, and LCDR3 comprise amino acids 23-32, 48-54, and 87-92 of SEQ ID NO: 21 (VRC13e VL), respectively;
(F) and the LCDR1, LCDR2, and LCDR3 comprise amino acids 23-32, 48-54 and 87-92 of SEQ ID NO: 22 (VRC13f VL), respectively;
(G) and the LCDR1, LCDR2, and LCDR3 comprise amino acids 23-30, 46-52, and 85-90 of SEQ ID NO: 23 (VRC13g VL), respectively:
(H) and the LCDR1, LCDR2, and LCDR3 comprise amino acids 23-30, 46-52, and 85-90 of SEQ ID NO: 24 (VRC13h VL), respectively
(I) and the LCDR1, LCDR2, and LCDR3 comprise amino acids 23-30, 46-52, and 85-90 of SEQ ID NO: 25 (VRC14 VL), respectively;
(J) and the LCDR1, LCDR2, and LCDR3 comprise amino acids 23-30, 46-52, and 85-90 of SEQ ID NO: 26 (VRC14b VL), respectively;
(K) and the LCDR1, LCDR2, and LCDR3 comprise acids 23-30, 46-52, and 85-90 of SEQ ID NO 27 (VRC14c VL), respectively;
(L) and the LCDR1, LCDR2, and LCDR1 comprise amino acids 23-30, 46-52, and 85-90 of SEQ ID NO: 28 (VRC15 VL), respectively
(M) and the LCDR1, LCDR2, and LCDR3 comprise amino acids 24-34, 50-56, and 89-97 of SEQ ID NO: 33 (VRC16), respectively;
(N) and the LCDR1, LCDR2 and LCDR3 corn rise amino acids 24-34, 50-56, and 89-97 of SEQ ID NO: 34 (VRC16b), respectively;
(O) and the LCDR1, LCDR2, and LCDR3 comprise acids 24-34, 50-56, and 89-97 of SEQ ID NO: 35 (VRC16c), respectively; or
(P) and the LCDR1, LCDR2 and LCDR3 comprise amino acids 24-34, 50-56 and 89-97 of SEQ ID NO: 36 (VRC16d), respectively.

10. The antibody or antigen binding fragment of claim 1, wherein the antibody or antibody or antigen binding fragment comprises the HCDR1, the HCDR2, and the HCDR3, according to:
(A) and the heavy chain variable region comprises the amino acid sequence set forth as SEQ ID NO: 5 (VRC13);
(B) and the heavy chain variable region comprises the amino acid sequence set forth as SEQ ID NO: 6 (VRC13b);
(C) and the heavy chain variable region comprises the amino acid sequence set forth as SEQ ID NO: 7 (VRC13c);
(D) and the heavy chain variable region comprises the amino acid sequence set forth as SEQ ID NO: 8: (VRC13d);
(E) and the heavy chain variable region comprises the amino acid sequence set forth as SEQ ID NO: 9 (VRC13e);
(F) and the heavy chain variable region comprises the amino add sequence set forth as SEQ ID NO: 10 (VRC13f);
(G) and the heavy chain variable region comprises the amino acid sequence set forth as SEQ ID NO: 11 (VRC13g);
(H) and the heavy chain variable region comprises the amino acid sequence set forth as SEQ ID NO: 12 (VRC13h);
(I) and the heavy chain variable region comprises the amino acid sequence set forth as SEQ ID NO: 13 (VRC14);
(J) and the heavy chain variable region comprises the amino acid sequence set forth as SEQ ID NO: 14 (VRC14b);
(K) and the heavy chain variable region comprises the amino acid sequence set forth as SEQ ID NO: 15 (VRC14c);
(L) and the heavy chain variable region comprises the amino acid sequence set forth as SEQ ID NO: 16 (VRC15);
(M) and the heavy chain variable region comprises the amino acid sequence set forth as SEQ ID NO: 29 (VRC16);
(N) and the heavy chain variable region comprises the amino acid sequence set forth as SEQ ID NO: 30 (VRC16b);
(O) and the heavy chain variable region comprises the amino acid sequence set forth as SEQ ID NO: 31 (VRC16c); or
(P) and the heavy chain variable region comprises the amino acid sequence set forth as SEQ ID NO: 32 (VRC16d).

11-14. (canceled)

15. The antibody or antigen binding fragment of claim 1, wherein the heavy chain variable region and the light chain variable region comprise the amino acid sequences set forth as:
(A) SEQ ID NO: 5 and SEQ ID NO: 17, respectively;
(B) SEQ ID NO: 6 and SEQ ID NO: 18, respectively;
(C) SEQ ID NO: 7 and SEQ ID NO: 19, respectively;
(D) SEQ ID NO: 8 and SEQ ID NO: 20, respectively;
(E) SEQ ID NO: 9 and SEQ ID NO: 21, respectively;
(F) SEQ ID NO: 10 and SEQ ID NO: 22, respectively;
(G) SEQ ID NO: 11 and SEQ ID NO: 23, respectively;
(H) SEQ ID NO: 12 and SEQ ID NO: 24, respectively;
(I) SEQ ID NO: 13 and SEQ ID NO: 25, respectively;
(J) SEQ ID NO: 14 and SEQ ID NO: 26, respectively;
(K) SEQ ID NO: 15 and SEQ ID NO: 27, respectively;
(L) SEQ ID NO: 16 and SEQ ID NO: 28, respectively.
(M) SEQ ID NO: 29 and SEQ ID NO: 33, respectively;
(N) SEQ ID NO: 30 and SEQ ID NO: 34, respectively;
(O) SEQ ID NO: 31 and SEQ ID NO: 35, respectively; or
(P) SEQ ID NO: 32 and SEQ ID NO: 36, respectively.

16. An isolated monoclonal antibody, or antigen binding fragment thereof, comprising a heavy chain variable region and a light chain variable region, wherein the heavy chain variable region comprises an amino acid sequence at least 80% identical to SEQ ID NO: 5, wherein the antibody specifically binds gp120 and is neutralizing.

17. The antibody or antigen binding fragment of claim 16, wherein the light chain variable region is at least 80% identical to SEQ ID NO: 17.

18. The antibody or antigen binding fragment of claim 16, wherein the antibody comprises a VH1-69 and VJ-2 germline heavy chain variable region.

19. The antibody or antigen binding fragment of claim 16, wherein the heavy chain variable region is a clonal variant from donor 44, and wherein the heavy chain is encoded by VH1-69 V gene, and a VJ-2 J gene from donor 44.

20. The antibody or antigen binding fragment of claim 16, wherein the antibody comprises a LV2-14 and a LJ-1 germline light chain variable region.

21. The antibody or antigen binding fragment of claim 16, wherein the light chain variable domain is a clonal variant from donor 44, with wherein the light chain is encoded by a LV2-14 V gene and a LJ-1 J gene.

22. An isolated monoclonal antibody, or antigen binding fragment thereof, comprising a heavy chain variable region and a light chain variable region, wherein
the heavy chain variable region comprises a heavy chain complementarity determining region (HCDR) 1, a HCDR2, and a HCDR3 of the amino acid sequence set forth as SEQ ID NO: 3;
the light chain variable region comprises a light chain complementarity determining region (LCDR) 1, a LCDR2, and a LCDR3, of the amino acid sequence set forth as SEQ ID NO: 4; and
wherein the antibody specifically binds to an epitope on the surface of gp120 and is neutralizing.

23-37. (canceled)

38. The antibody of claim 1, wherein the antibody is an IgG, IgM or IgA.

39. A bispecific antibody comprising the antibody or antigen binding fragment of claim 1.

40. The bispecific antibody of claim 39, comprising:
a first antigen binding domain comprising the antigen binding fragment of claim 1, wherein the antigen binding fragment is a Fab or an scFv; and
a second antigen binding domain comprising a second antigen binding fragment that specifically binds to an antigen other than gp120, wherein the second antigen binding fragment is a Fab or scFv.

41. The antigen binding fragment of claim 1, wherein the fragment is a Fab fragment, a Fab′ fragment, a F(ab)′₂ fragment, a single chain Fv protein (scFv), or a disulfide stabilized Fv protein (dsFv).

42. The antigen binding fragment of claim 41, wherein the antigen binding fragment is a Fab or an scFv fragment.

43. The antibody or antigen binding fragment of claim 1, linked to an effector moiety.

44. The antibody or antigen binding fragment of claim 43, wherein the effector moiety is a toxin or a detectable label.

45. The antibody or antigen binding fragment of claim 44, wherein the label is a fluorescent, enzymatic, or radioactive label.

46. A scaffold protein comprising the antigen binding fragment of claim 1.

47. An isolated nucleic acid molecule encoding the antibody or antigen binding fragment of claim 1.

48. The nucleic acid molecule of claim 47, operably linked to a promoter.

49. An expression vector comprising the nucleic acid molecule of claim 48.

50. An isolated host cell transformed with the nucleic acid molecule of claim 47.

51. A composition comprising a therapeutically effective amount of the antibody or antigen binding fragment of claim 1 or a nucleic acid molecule encoding the antibody or anti binding fragment and a pharmaceutically acceptable carrier.

52. A method of detecting a human immunodeficiency virus (HIV)-1 infection in a subject, comprising:
contacting a biological sample from the subject with the antibody or antigen binding fragment of claim 1 under conditions sufficient to form an immune complex; and
detecting the presence of the immune complex on the sample from the subject, wherein the presence of the immune complex on the sample from the subject indicates that the subject has an HIV-1 infection.

53. The method of claim 52, wherein the antibody or antigen binding fragment is directly labeled.

54. The method of claim 52, wherein the contacting is in vivo.

55. The method of claim 52, wherein the contacting is in vitro.

56. The method of claim 52, further comprising:
contacting the sample with a second antibody that specifically binds to the antibody or the antigen binding fragment; and
detecting the binding of the second antibody to the sample;
wherein an increase in binding of the second antibody to the sample as compared to binding of the second antibody to a control sample detects the presence of an HIV-1 infection the subject.

57. A method for preventing or treating a human immunodeficiency virus (HIV)-1 infection in a subject, comprising administering to the subject a therapeutically effective amount of the antibody or antigen binding fragment of claim 1 or a nucleic acid molecule encoding the antibody or antigen binding fragment, thereby preventing or treating the HIV-1 infection.

58. The method of claim 57, wherein the method is a method for treating a HIV-1 infection, and wherein the subject has acquired immune deficiency syndrome (AIDS).

59. The method of claim 57, further comprising administering to the subject an additional anti-viral agent.

60. The method of claim 59, wherein the additional antiviral agent comprises a nucleoside analog reverse-transcriptase inhibitor, a nucleotide reverse transcriptase inhibitor, a non-nucleoside reverse transcriptase inhibitor, a protease inhibitor, an entry or fusion inhibitor, a maturation inhibitor, or a broad spectrum inhibitor or a combination thereof.

61. The method of claim 57, further comprising administering to the subject one or more additional antibodies, or nucleic acids encoding such antibodies, wherein the additional antibodies specifically bind gp120 and/or gp4l.

62. The method of claim 57, further comprising measuring HIV-1 viral titer in the subject.

63. A kit comprising:
(a) the antibody or antigen binding fragment of claim 1 or a nucleic acid molecule encoding the antibody or antigen binding fragment; and
(b) instructions for using the kit.

64. (canceled)

65. An isolated monoclonal antibody, or antigen binding fragment thereof, comprising
a heavy chain variable region comprising a heavy chain complementarity determining region (HCDR) 1, a HCDR2, and a HCDR3 of the amino acid sequence set forth as SEQ ID NO: 1;
wherein the antibody specifically binds to gp120 and is neutralizing.

66. The antibody or antigen binding fragment of claim 65, further comprising a light chain variable region comprising a light chain complementarity determining region (LCDR) 1, a LCDR2, and a LCDR3 of the amino acid sequence set forth as SEQ ID NO: 4.

67. The antibody or antigen binding fragment of claim 1, further comprising a Fc polypeptide comprising M428L and N434S mutations.

68. The expression vector of claim 49, wherein the expression vector is an Adeno-associated viral vector.
